(19) United States
(12) Patent Application Publication
(10) Pub. No.: US 2024/0209371 A1
(43) Pub. Date: Jun. 27, 2024

(54) COMPOSITIONS AND METHODS FOR TREATING CANCER

(71) Applicant: Dana-Farber Cancer Institute, Inc., Boston, MA (US)

(72) Inventors: Pasi A. Jänne, Needham, MA (US); Yoshihisa Kobayashi, Brookline, MA (US)

(21) Appl. No.: 18/287,835

(22) PCT Filed: Apr. 22, 2022
(86) PCT No.: PCT/US22/25920

§ 371 Date: Oct. 20, 2023

Related U.S. Application Data
(60) Provisional application No. 63/178,150, filed on Apr. 22, 2021.
(60) Provisional application No. 63/292,842, filed on Dec. 22, 2021.

(51) Int. Cl. C12N 15/113; A61K 45/06; A61P 35/00

(52) U.S. Cl. CPC C12N 15/1137; A61K 45/06; A61P 35/00; C12N 15/1135; C12N 2310/11; C12N 2310/315; C12N 2310/321; C12N 2310/3233; C12N 2320/33

(57) ABSTRACT

This invention comprises compositions and methods for treating cancer. For example, this invention comprises compositions and methods for treating cancer by modulating splicing.

This application is an International Application, which claims the benefit of priority from U.S. provisional patent application no. 63/178,150 filed on Apr. 22, 2021 and U.S. provisional patent application No. 63/292,842 filed on Dec. 22, 2021, the entire contents of which is incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### FIELD OF THE INVENTION

This invention is directed to compositions and methods for treating cancer. For example, this invention is directed to compositions and methods for treating cancer by modulating splicing.

### BACKGROUND OF THE INVENTION

The RAS family includes the homologous proteins NRAS, HRAS, and KRAS (Parikh et al, Blood 108:1, 2006). The RAS family members encode 21-kD guanine nucleotide binding proteins that operate as molecular switches to regulate the transduction of physiological signals from the cell membrane to the nucleus (Mackenzie et al, Blood 93:6, 1999). The RAS family members have been implicated in cancer.

### SUMMARY OF THE INVENTION

Aspects of the invention are drawn to nucleic acid comprising a sequence that hybridizes to a target, wherein the target comprises a precursor-mRNA (pre-mRNA) of a RAS family gene, wherein the precursor-mRNA comprises an exon splicing enhancer (ESE) binding motif comprising at least one mutation. For example, the RAS family gene comprises KRAS, NRAS, and HRAS.

In embodiments, the nucleic acid modulates splicing of the pre-mRNA. For example, the nucleic acid promotes splicing or inhibits splicing of the pre-mRNA.

In embodiments, the target comprises exon 3 of the pre-mRNA or portion thereof. For example, the nucleic acid promotes alternative splicing which excludes exon 3 or a portion thereof.

In embodiments, the sequence of the nucleic acid is at least partially complementary to, partially complementary to, or fully complementary to the target.

In embodiments, the target comprises at least one mutation as compared to wildtype pre-mRNA.

In embodiments, the sequence is not complementary to wildtype pre-mRNA.

In embodiments, the target comprises the exon splicing enhancer (ESE) binding motif, or a sequence adjacent thereto.

For example, the exon splicing enhancer (ESE) binding motif comprises codons 52 to 70 of the pre-mRNA or a portion thereof.

For example, the exon splicing enhancer (ESE) binding motif comprises codons 55 to 65 of pre-mRNA of KRAS, or a portion thereof.

For example, the exon splicing enhancer (ESE) binding motif comprises codons 55 to 66 of pre-mRNA of NRAS.

For example, the exon splicing enhancer (ESE) binding motif comprises codons 58 to 67 pre-mRNA of HRAS.

In embodiments, the target comprises a sequence comprising

[SEQ ID NO: [ ]]

5′-CUC UUG GAU AUU CUC GAC ACA GCA GGX₁ X₂X₃X₄
GAG GAG UAC AGU GCA AUG AGG GAC CAG-3′

or at least 90% identical thereto. For example,
X₁ is G, A, U, or C;
X₂ is G, A, U, or C;
X₃ is G, A, U, or C;
X₄ is G, U, A, or C; or
any combination thereof.

In embodiments, the target comprises a nucleic acid sequence comprising:

| [SEQ ID NO: [ ]] | |
|---|---|
| | 5′-CUC UUG GAU AUU CUC GAC ACA GCA GGU CUA GAG |
| | GAG UAC AGU GCA AUG AGG GAC CAG-3′, |
| [SEQ ID NO: [ ]] | |
| | 5′-CUC UUG GAU AUU CUC GAC ACA GCA GGU CAC GAG |
| | GAG UAC AGU GCA AUG AGG GAC CAG-3′, |
| [SEQ ID NO: [ ]] | |
| | 5′-CUC UUG GAU AUU CUC GAC ACA GCA GGG AAA GAG |
| | GAG UAC AGU GCA AUG AGG GAC CAG-3′, |
| | and |
| [SEQ ID NO: [ ]] | |
| | 5′-CUC UUG GAU AUU CUC GAC ACA GCA GGC AAA GAG |
| | GAG UAC AGU GCA AUG AGG GAC CAG-3′, |

or a sequence at least 90% identical thereto.

In embodiments, the target comprises a nucleic acid sequence comprising

[SEQ ID NO: [ ]]
5′-UUG UUG GAC AUA CUG GAU ACA GCU GGA X₁X₂A GAA
GAG UAC AGU GCC AUG AGA GAC CAA-3′,

or a sequence at least 90% identical thereto. For example,
X₁ is G, U, A, or C;
X₂ is G, U, A, or C; or
any combination thereof.

In embodiments, the target comprises a nucleic acid sequence comprising:

[SEQ ID NO: [ ]]
5′-UUG UUG GAC AUA CUG GAU ACA GCU GGA CGA GAA
GAG UAC AGU GCC AUG AGA GAC CAA-3′,
[SEQ ID NO: [ ]]
5′-UUG UUG GAC AUA CUG GAU ACA GCU GGA AAA GAA
GAG UAC AGU GCC AUG AGA GAC CAA-3′,
[SEQ ID NO: [ ]]
5′-UUG UUG GAC AUA CUG GAU ACA GCU GGA CUA GAA
GAG UAC AGU GCC AUG AGA GAC CAA-3′,

or a sequence at least 90% identical thereto.

In embodiments, the target comprises a nucleic acid sequence comprising:

[SEQ ID NO: [ ]]
5′-CUG UUG GAC AUC CUG GAU ACC GCC GGC CXX GAG
GAG UAC AGC GCC AUG CGG GAC CAG-3′,

or a sequence at least 90% identical thereto. For example,
X₁ is G, U, A, or C;
X₂ is G, U, A, or C; or
any combination thereof.

In embodiments, the target comprises a nucleic acid sequence comprising:

[SEQ ID NO: [ ]]
5′-CUG UUG GAC AUC CUG GAU ACC GCC GGC CUG GAG
GAG UAC AGC GCC AUG CGG GAC CAG-3′,
[SEQ ID NO: [ ]]
5′-CUG UUG GAC AUC CUG GAU ACC GCC GGC CAU GAG
GAG UAC AGC GCC AUG CGG GAC CAG-3′,

or a sequence at least 90% identical thereto.

In embodiments, the target nucleic acid comprises at least one mutation, such as a mutation within codon 61. For example, the at least one mutation comprises G60X, Q61X, or a combination thereof. For example, the at least one mutation comprises Q61H, Q61L, Q61R, or Q61K. For example, the at least one mutation comprises GQ60GK.

In embodiments, the nucleic acid comprises a sequence according to

[SEQ ID NO: [ ]]
5′-GTACTCCTCX₁X₂X₃X₄CCTGCTGTGTCG-3′,

or

a sequence that is at least 90% identical thereto. For example,
X₁ is G or T;
X₂ is A or T;
X₃ is G or T;
X₄ is G, C, or A; or
any combination thereof.

In embodiments, the nucleic acid comprises a sequence of

[SEQ ID NO: [ ]]
5′-GTACTCCTCTTTGCCTGCTGTGTCG-3′,
[SEQ ID NO: [ ]]
5′-GTACTCCTCTTTCCCTGCTGTGTCG-3′,
[SEQ ID NO: [ ]]
5′-GTACTCCTCGTGACCTGCTGTGTCG-3′,
[SEQ ID NO: [ ]]
5′-GTACTCCTCTAGACCTGCTGTGTCG-3′,

or

a sequence that is at least 90% identical thereto.

In embodiments, the nucleic acid comprises a sequence according to 5′-CTTX₁X₂CCTGCTGTGTCGAGA-3′ [SEQ ID NO: [ ]], or a sequence that is at least 90% identical thereto. For example,
X₁ is G or T;
X₂ is G, C or A; or

any combination thereof.

In embodiments, the nucleic acid comprises a sequence of

[SEQ ID NO: [ ]]
5′-CTTTGCCTGCTGTGTCGAGA-3′,
[SEQ ID NO: [ ]]
5′-CCTCTTTGCCTGCTGTGTCG-3′,
[SEQ ID NO: [ ]]
5′-ACTCCTCTTTGCCTGCTGTG-3′,
[SEQ ID NO: [ ]]
5′-TGTACTCCTCTTTGCCTGCT-3′,
[SEQ ID NO: [ ]]
5′-CACTGTACTCCTCTTTGCCT-3′,
or
[SEQ ID NO: [ ]]
5′-TTGCACTGTACTCCTCTTTG-3′.

In embodiments, the nucleic acid comprises a sequence according to 5′-X₁X₂X₃X₄-3′, wherein X₁ is G or T; X₂ is A or T; X₃ is G or T; X₄ is G, C, or A; or any combination thereof. In embodiments, the nucleic acid further comprises 5′ flanking nucleotides, 3′ flanking nucleotides, or both 5′ flanking nucleotides and 3′ flanking nucleotides.

In embodiments, the nucleic acid comprises a sequence of

(SEQ ID NO: [ ])
5′-TAGACCTGCTGTGTCGAGAATATCC-3′,
(SEQ ID NO: [ ])
5′-CTCTAGACCTGCTGTGTCGAGAATA-3′,
(SEQ ID NO: [ ])
5′-CTCCTCTAGACCTGCTGTGTCGAGA-3′,
(SEQ ID NO: [ ])
5′-GTACTCCTCTAGACCTGCTGTGTCG-3′,
(SEQ ID NO: [ ])
5′-ACTGTACTCCTCTAGACCTGCTGTG-3′,
(SEQ ID NO: [ ])
5′-CATTGCACTGTACTCCTCTAGACCT-3′,
or
(SEQ ID NO: [ ])
5′-CCTCATTGCACTGTACTCCTCTAGA-3′.

In embodiments, the nucleic acid comprises a sequence of

[SEQ ID NO: [ ]]
5′-CTCTTCTX₁X₂TCCAGCTGTATCCAGT-3′,

a sequence that is at least 90% identical thereto. For example,
X₁ is C, T, or A;
X₂ is T or G;
or any combination thereof.

In embodiments, the nucleic acid comprises a sequence of

[SEQ ID NO: [ ]]
5′-CTCTTCTCGTCCAGCTGTATCCAGT-3′,
[SEQ ID NO: [ ]]
5′-CTCTTCTTTTCCAGCTGTATCCAGT-3′,
[SEQ ID NO: [ ]]
5′-CTCTTCTAGTCCAGCTGTATCCAGT-3′,

or

a sequence that is at least 90% identical thereto.

In embodiments, the nucleic acid comprises a sequence of

[SEQ ID NO: [ ]]
5′-TGGCGCTGTACTCCTCX₁X₂GGCCGG-3′,

or

a sequence that is at least 90% identical thereto. For example,
X₁ is A or C;
X₂ is T or A; or
any combination thereof.

In embodiments, the nucleic acid comprises a sequence of

[SEQ ID NO: [ ]]
5′-TGGCGCTGTACTCCTCCAGGCCGG-3′,
[SEQ ID NO: [ ]]
5′-TGGCGCTGTACTCCTCATGGCCGG-3′,

or

a sequence that is at least 90% identical thereto.

In embodiments, the nucleic acid comprises a modified nucleic acid. For example, the modified nucleic acid comprises a sugar modification, a backbone modification, a base modification, an unnatural base pair, conjugation to a cell penetrating peptide, or any combination thereof. For example, the modification comprises phosphorothioate (PS)+2′-O-Methyoxyethyl (2′MOE).

In embodiments, the modified nucleic acid is a morpholino, a locked nucleic acid (LNA), a cell penetrating peptides-conjugated morpholino amido-bridged nucleic acid (AmNA), or peptide nucleic acid (PNA).

Aspects of the invention are further drawn to a composition, such as a composition comprising a nucleic acid as described herein.

In embodiments, the composition further comprises a pharmaceutically acceptable carrier, diluent, or excipient.

In embodiments, the composition further comprises at least one additional active agent. For example, the at least one additional active agent comprises an anti-cancer agent.

Still further, aspects of the invention are drawn to a method for treating cancer in a subject in need thereof. For example, the method comprises administering to the subject a therapeutically effective amount of a nucleic acid as described herein or a composition described herein.

In embodiments, the cancer comprises a RAS-associated cancer. For example, the RAS is KRAS, NRAS, or HRAS.

In embodiments, the RAS-associated cancer comprises at least one mutation in KRAS, NRAS, or HRAS. For example, the at least one mutation comprises G60X, Q61X, or a combination thereof. For example, the at least one mutation comprises Q61H, Q61L, Q61R, or Q61K. For example, the at least one mutation comprises GQ60GK.

Also, aspects of the invention are drawn to a method for inducing splicing in a subject. For example, the method comprises administering to the subject a therapeutically effective amount of a nucleic acid as described herein or a composition as described herein.

Still further, aspects of the invention are drawn to a method for modulating splicing of a RAS pre-mRNA in a tumor cell. For example, the method comprises administering to the subject a therapeutically effective amount of a nucleic acid as described herein or a composition as described herein. For example, the method comprising contacting the tumor cell with the nucleic acid as described herein, such as in an amount effect to modulate splicing of the RAS pre-mRNA in the tumor cell.

In embodiments, modulating splicing comprises enhancing or inducing splicing. For example, modulating splicing comprises modulating splicing of Exon 3.

In embodiments, splicing is modulated in tumor cells but not in normal cells.

Further, aspects of the invention are drawn to methods for inducing skipping of an exon in a tumor cell. For example, embodiments comprise contacting the tumor cell with the nucleic acid as described herein, such as in an amount effective to induce skipping of an exon of in the tumor cell.

In embodiments, the exon comprises an exon of RAS, for example exon 3. For example, RAS comprises KRAS, NRAS, or HRAS.

Aspects of the invention are also drawn towards a kit comprising a nucleic acid as described herein and/or a composition as described herein, and instructions for use thereof.

Other objects and advantages of this invention will become readily apparent from the ensuing description.

### BRIEF DESCRIPTION OF THE FIGURES

FIG. 1 shows KRAS Q61K imparts resistance to osimertinib only in the presence of a concurrent KRAS G60G silent mutation. Panel a. Colony formation assay, using the parental PC9 cell line, or CRISPR-Cas9-modified PC9 cell lines that express mutant KRAS or BRAF, following 1 or 3 weeks of treatment with osimertinib. Panel b. Under osimertinib selection pressure, allele frequencies (AFs) of the KRAS Q61K mutation increase only in the presence of a silent mutation at G60 (GQ60GK c.180_181 delinsCA or AA). Panel c. Representative sequencing chromatograms of KRAS DNA derived from single clones with indicated mutations. Panel d. Cell viability assay of PC9 cells in the absence or presence of different KRAS or BRAF mutants after 72 hours of osimertinib treatment. P-values were calculated by ANOVA, followed by Dunnett's post-hoc test, **p<0.01. Panel e. AFs of KRAS GQ60GK (c.180_181 delinsGA) and Q61H, but not KRAS G60G, increase during osimertinib selection. Panel f. Cell viability assay of modified PC9 cells after 72 hours of osimertinib treatment. GQ60GK c.180_181 delinsAA and Q61K were homozygous, whereas Q61H was heterozygous. Panel g. Western blot analyses following osimertinib treatment demonstrate persistent ERK activation in PC9 cells expressing KRAS GQ60GK c.180_181 delinsAA but not in cells expressing KRAS Q61K alone. Panel h. RAS-GTP assay in KRAS expressing PC9 cell lines was performed following a 24-hour treatment with or without 1 μM osimertinib. Panel i. Knockdown of KRAS or BRAF genes by siRNA resensitize different PC9 cell line models to osimertinib. KRAS GQ60GK c.180_181 delinsAA is homozygous and others are heterozygous. P-values were calculated by Student's t test, *p<0.05, **p<0.01.

FIG. 2 shows KRAS Q61K co-occurs with G60G silent mutation in 3 independent pan-cancer cohorts. Panel a. Non-synonymous and silent mutations in KRAS, NRAS, and HRAS genes obtained from The Cancer Genome Atlas (TCGA) pan-cancer cohort. Pie charts include all non-synonymous and silent mutations in each gene. Non-synonymous mutations at Q61, and silent mutations, are shown in bar charts. The frequency of co-occurrence of activating non-synonymous Q61X and the G60G silent mutation was evaluated by Fisher's exact test, **p<0.01. Background activating mutations that coexist with silent mutations are shown in FIG. 15. Data on cancer type and the presence of the G60G silent mutation at c. 180 are shown in tumors with RAS G60G. Panel b. Pan-cancer cohort (n=25,252) evaluated by targeted-next generation sequencing (NGS). 23 cases with KRAS Q61K had a co-occurring G60G silent mutation. The correlation between allele frequencies (AFs) of Q61K and G60G was analyzed by Pearson's correlation coefficient. Panel c. Venn diagram showing the distribution of KRAS Q61K, G60G, and Q61H mutations in the Guardant Health cohort, detected by targeted NGS using cell free DNA. The co-occurrence of activating non-synonymous and G60G silent mutation was evaluated by Fisher's exact test. Panel d. AFs of KRAS A59A silent mutation with or without Q61K, evaluated by next generation sequencing in PC9 models edited by CRISPR-Cas9, undergoing osimertinib treatment. Panel e. The correlation between AFs of KRAS Q61K and G60G/A59A in the cohort.

FIG. 3 shows silent mutation in KRAS G60G can be necessary for splicing of KRAS Q61K. Panel a. Images of KRAS-specific PCR amplicons of cDNA, generated from CRISPR-Cas9 modified PC9 clones expressing different KRAS mutations. Heterozygosity or homozygosity of KRAS mutants is shown for each clone. M: 100 bp-marker, m: mutant, wt: wild type, fs: frame-shift, Panel b. Schemas of different KRAS isoforms shown in Panel a. AA: amino acid. Panel c. Representative sequencing chromatograms of KRAS cDNA derived from isoforms that lack 112 bp of exon 3, or whole exon 3, which result in a subsequent frameshift and an early stop codon. Panel d. Comparison of the conserved motif of splicing donor site²³ and the DNA sequence of KRASINRASIHRAS around Q61. Nucleotides at c. 180 and c. 181, serving as putative cryptic splice donor sites, are shaded in blue. Nucleotide changes, including deletions, are shown as red vertical bars. Consensus values of splice site were estimated by Human Splicing Finder²⁴. KRAS mutants with cryptic splice donor sites and their consensus values are shown in blue. AS: alternative splicing, *not report Panel e. Western blot analysis in KRAS expressing PC9 cell lines was performed following 1 μM osimertinib with antibodies targeting N-terminal or C-terminal epitopes of KRAS. Panel f. Exonic splicing enhancers (ESE) and silencers (ESS) around KRASINRASIHRAS Q61 were simulated using the Human Splicing Finder. Threshold values indicate the strength of each motif. Matrices for SR proteins including SF2/ASF, SRp40, SC35, and SRp55 were obtained from the ESE Finder tool^{38,39}. The RESCUE-ESE hexamers⁴⁰ and the putative octamer ESE⁴¹ are also shown. The relative strength of ESE/ESS octamers is denoted by a curved yellow line.

FIG. 4 shows antisense oligo induces aberrant splicing in RAS Q61X cancers and leads to therapeutic effects in vitro and in vivo. Panel a. Schema of treatment strategy: antisense oligo nucleotides hybridize to the exonic splicing enhancer motifs in pre-mRNA of KRASINRASIHRAS Q61 cancer, but not their wild type counterparts, induce skipping of exon 3, resulting in premature termination. Panel b. Images of KRAS-specific PCR amplicons generated from the cDNA of Calu6 that was treated with increasing concentrations of KRAS GQ60GK-selective or control morpholino oligos for 48 hours. Exon 3 skipping fraction is defined as the band intensities of “skipped/(skipped+full-length)” transcript as quantified by ImageJ. M: 100 bp-marker. P-values were calculated between cells treated with targeted morpholino and those with mor-CTRL using Student's t test, *p<0.05, **p<0.01. Panel c. Images of PCR amplicons generated from the cDNA of KRASINRASIHRAS Q61 mutant cell lines treated with 10 μM morpholino for 48 hours. CTRL: control, mor-1,4,6,9: morpholino targeting KRAS GQ60GK, KRAS Q61L, NRAS Q61K, and HRAS Q61H, respectively. P-values were calculated using the same method as in panels b. Panel d. Transcript frequencies of KRAS Q61L versus wild type in the intact full-length KRAS amplicon derived from mRNA of SW948 cells treated with morpholinos. Panel e. Summary of relative RAS dependency in a panel of mutant RAS cell lines, and their sensitivity to treatment with or antisense oligos. Gene effect scores for dependency, evaluated by RNAi and CRISPR knock out, were obtained from Depmap (depmap.org/portal/), where a score of 0 is equivalent to a gene that is not essential whereas a score of −1 corresponds to the median of all common essential genes. Scores are shown in heat map: <−1 in blue, 0<in red, and others in yellow. Cell viability assays in suspension cells after 8 days of siRNA (top) and 10 μM morpholino (bottom) treatment were performed. In the bar chart of morpholino treatment, colors indicate each mutant-selective morpholinos. Panel f. The correlation of growth inhibition by RAS siRNA and morpholino antisense oligo nucleotide in 17 RAS Q61X cell lines. Panel g. Western blot analyses of signaling in KRAS mutant cell lines were performed after 72 hour treatment with 10 nM trametinib, 10 μM morpholino or respective controls. Panel h. In vivo imaging of luciferase-expressing H650 xenograft tumors. H650 cells were pre-treated with 10 μM vivoMor-CTRL or vivoMor-4 in vitro for 1 or 2 days prior to injection into nude mice. In vivo imaging was performed twice a week (n=10 per treatment group). Panel i. Representative Images of pre-treated H650 xenograft tumors. Panel j. In vivo efficacy of H650 xenograft tumors treated with daily intra-tumoral injection of morpholino (n=10 per treatment group).

FIG. 5 shows CRISPR-Cas9 modified EGFR mutant PC9 cells for evaluating oncogenicity of KRAS or BRAF mutations. Panel a. A schema of selection with EGFR inhibitor osimertinib. Majority of parental EGFR-mutant PC9 cells are sensitive to osimertinib with only a fraction exhibiting intrinsic resistance. In bulk CRISPR-Cas9 modified PC9 cells, osimertinib treatment can lead to an increase in the fraction of cells harboring a resistance mutation such as KRAS G12C. Panel b. Cell viability assay of parental and CRISPR-Cas9 modified PC9 cells after 72 hours of osimertinib treatment. Each clone has heterozygous KRAS mutations: GQ60GK c.180_181 delinsCA plus Q61K, GQ60GK c.180_181 delinsGA plus frameshift, and Q61K plus frameshift. Panel c. Knockdown of KRAS or BRAF in CRISPR-Cas9-modified PC9 clones following 48 hours of KRAS or BRAF specific siRNA treatment are shown by western blot analyses.

FIG. 6 shows alternative splicing of KRAS in CRISPR-Cas9 modified PC9 cells. Panel a. Images of KRAS-specific PCR amplicons of cDNA, generated from CRISPR-Cas9 modified PC9 clones expressing different KRAS mutations in the presence or absence of osimertinib given the influence of upstream EGFR signals. Heterozygosity or homozygosity of KRAS mutants is shown for each clone. M: 100 bp-marker, m: mutant, wt: wild type, fs: frameshift. Panel b. Images of KRAS-specific PCR amplicons of cDNA, generated from additional CRISPR-Cas9 modified PC9 clones expressing different KRAS mutations. Panel c. shows images of KRAS-specific PCR amplicons of cDNA, generated from KRAS mutant cell lines and CRISPR-Cas9 modified PC9 clones expressing different KRAS mutations.

FIG. 7 shows a strategy to convert the original KRAS GQ60GK into the non-functional Q61K by editing silent mutations using CRISPR-Cas9. Panel a. A schema of the proposed alternative treatment strategy using CRISPR-Cas9 editing with a mutant-specific sgRNA substituting the c. 180 silent mutation with a cryptic splice donor nucleotide in order to promote exonic skipping leading to a premature STOP codon Panel b. Allele frequencies of mutations, evaluated by next generation sequencing using DNA derived from bulk KRAS GQ60GK-mutant CALU6 and SNU668 cell lines, 48 hours after CRISPR-Cas9 editing with indicated donor templates. AF of original KRAS GQ60GK decreased by CRISPR editing with GQ60GK-specific sgRNA and AF of non-functional Q61K increased only in the presence of donor template designed for Q61K. Panel c. Relative expression of indicated KRAS isoforms, evaluated by qPCR in KRAS GQ60GK-mutant CALU6 and SNU668 cell lines, 48 hours after CRISPR-Cas9 editing with indicated donor templates. Expression data are shown as relative to cell lines using Q61K template. Although it can be difficult to capture clones successfully converted to non-functional Q61K due to their inability to grow, increased expression level of the KRAS isoform skipping 112 bp was confirmed in the remaining cells 2 days after CRISPR-editing.

FIG. 8 shows a scheme of designing mutant-selective antisense oligos. Panel a. Structures of DNA, antisense oligo nucleotides with phosphorothioate (PS)+2′-O-Methoxyethyl (2′MOE) modifications, and morpholino. In PS+2′MOE, a non-bridging oxygen is replaced by a sulfur atom in the phosphate backbone, and 2′ position of the sugar moiety is modified. In morpholino, the sugar moiety is replaced with methylenemorpholine rings, and the anionic phosphates are replaced with non-ionic phosphorodiamidate linkages. Panel b. Schema depicting the design of the KRAS GQ60GK c.180_181 delinsCA, NRAS Q61K, and HRAS Q61L selective antisense oligo. The motifs for binding exonic splicing enhancers simulated using the Human Splicing Finder (top) and ESE finder (bottom) are shown. Matrices for SR proteins including SRSF1 (SF2/ASF and IgM-BRCA1), SRSF2 (SC35), SRSF5 (SRp40), and SRSF6 (SRp55) are shown.

FIG. 9 shows mutant selective inhibition of RAS using morpholino. Raw NGS reads showing KRAS Q61L transcript were visualized using IGV software.

FIG. 10 shows in vitro sensitivity to MEK inhibitor and morpholino oligos in RAS mutant cells Panel a. Cell viability assay in suspension cells after 8 days of 10 μM morpholino treatment were performed on ultra-low attachement plates. P-values were calculated by ANOVA, followed by Dunnett's post-hoc test compared with cells treated with DMSO, **p<0.01. Panel b. Cell viability assay of a panel of mutant RAS cell lines after 72 hours of trametinib treatment in 2D adherent or 3D suspension culture. Panel c. The correlation of growth inhibition by 10 nM trametinib in 2D or 3D culture and morpholino antisense oligo nucleotide in 17 RAS Q61X cell lines. Panel d, Western blot analyses of signaling in KRAS wild type cell lines were performed after 72 hours treatment with 10 nM trametinib, 10 μM morpholino or respective controls. Panel e. Relative expression of ERK signature genes, evaluated by qPCR in CALU6 and H650 cell lines treated with mutant-selective morpholino for 48 hours. Expression data are normalized to readout of a control morpholino treatment. GUSB was used as an internal control. P-values were calculated by Student's t test, **p<0.01. Panel f. Cell viability assays in suspension cells after 8 days of 50 nM afatinib, 10 μg/ml cetuximub, and 10 μM morpholino treatment with the same method as Panel a. Panel g. Western blot analyses of signaling in NRAS and HRAS mutant cell lines were performed after 72 hours of treatment with 10 nM trametinib, 10 μM morpholino, or DMSO.

FIG. 11 show pre-treatment strategy using vivo-morpholino in vitro and in vivo H650 models Panel a, Morpholino oligo pre-treatment strategy. H650 cells were pre-treated with 10 μM control vivo-morpholino and target vivo-morpholino for 1 to 4 days as 3D suspension cells. After drug washout, cells were cultured in growth media and cell viability was evaluated on day 8 in vitro. Luciferase-expressing H650 cells were used for in vivo experiments. After pre-treatment with 10 μM vivoMor-CTRL and target vivo-morpholino for 1 to 2 days as 3D suspension cells, drugs were washed out and cells were subcutaneously implanted into mice. In vivo imaging was performed twice a week. Panel b, In vitro cell viability assays of H650 cells pre-treated with 10 μM vivoMor-CTRL or vivoMor-4 for 1 to 4 days. P-values were calculated by Student's t test, **p<0.01. Panel c, Volume change of pre-treated H650 xenograft tumors. H650 cells were pre-treated with vivoMor-CTRL or vivoMor-4 in vitro for 1 or 2 days prior to injection into nude mice. Data are mean±S.E. (n=10).

FIG. 12 shows intra-tumoral injection of vivo-morpholino in H650 xenograft models Panel a. Images of KRAS-specific PCR amplicons generated from the cDNA of H650 xenograft tumors that were treated with daily intra-tumoral injection of morpholino for 7 days. Fraction of exon 3 skipping is defined as the band intensities of “skipped/(skipped+full-length)” as measured by ImageJ. M: 100 bp-marker. Panel b. Fractions of exon 3 skipping in samples shown in Panel (a) were statistically compared by Student's t test, **p<0.01. Panel c. Relative expression of KRAS exon 3 skipping, evaluated by qPCR in the tumor samples corresponding to (a). Panel d. Body weight change of mice with H650 xenograft tumors treated with morpholino over time.

FIG. 13 shows Pre-treatment strategy and intra-tumoral injection of vivo-morpholino in Calu6 models Panel a, In vitro cell viability assays of Calu6 cells pre-treated with 10 μM vivoMor-CTRL or vivoMor-1 for 1 to 4 days. P-values were calculated by Student's t test, **p<0.01. Panel b. Volume change of pre-treated Calu6 xenograft tumors. Calu6 cells were pre-treated with vivoMor-CTRL or vivoMor-1 in vitro for 24 hours prior to injection into nude mice (n=10). Panel c. In vivo efficacy of Calu6 xenograft tumors treated with daily intra-tumoral injection of morpholino (n=10). Panel d. Body weight change of mice with Calu6 xenograft tumors treated with morpholino over time.

FIG. 14 shows data indicating mutant selective inhibition of KRAS using PS+2′MOE antisense oligos. Panel a. Schema depicting the design of the KRAS GQ60GK c. 180_181delinsCA selective antisense oligo by screening. Panel b. Images of KRAS-specific PCR amplicons generated from the cDNA of cells treated with 6 kinds of PS+2′MOE antisense oligos for 48 hours. Exon 3 skipping fraction is defined as the band intensities of “skipped/(skipped+full length)” transcript as quantified by ImageJ. M: 100 bp-marker. Panel c. Images of KRAS-specific PCR amplicons in indicated cell lines with same method as panel b. Panel d. Transcript reads of KRAS GQ60GK or Q61L versus wild type in the intact full-length KRAS amplicon derived from mRNA of Calu6 and SW948 cells treated with PS+2′MOE antisense oligos. Panel e. Western blot analysis of signaling in KRAS mutant and wild type cell lines were performed after 6 days of 0.5 μM PS+2′MOE antisense oligos. Panel f. Cell viability assays in suspension cells after 8 days of 0.5 μM PS+2′MOE antisense oligos treatment were performed.

FIG. 15 shows silent mutations and background activating mutations in TCGA cohort.

FIG. 16 shows details of allele frequency of KRAS Q61K and G60G in DFCI internal cohort.

FIG. 17 shows co-mutations of 4 KRAS G60G cases without Q61K in Guardant Health cohort.

FIG. 18 shows exon 3 skipping at baseline in TCGA cohort.

FIG. 19 shows sequences of target morpholinos and control morpholino.

FIG. 20 shows binding affinity of morpholinos with mutant or wild type sequences.

FIG. 21 shows genome-wide screening of potential off-target binding sites.

FIG. 22 shows binding affinity of screeded PS+2′MOE oligos with mutant or wild type sequences.

FIG. 23 shows genome-wide screening of potential off-target binding sites of screened PS+2′MOE oligos.

FIG. 24 shows materials and methods described herein.

FIG. 25 (panels a, b, c, d, and e) shows in vitro treatment using morpholino.

FIG. 26 shows KRAS exon 3.

FIG. 27 shows NRAS exon 3.

FIG. 28 shows HRAS exon 3.

FIG. 29 shows demand for targeted therapy against RAS Q61 cancer.

FIG. 30 shows splicing vulnerabilities in KRAS Q61K mutant cancer. G60G silent mutation is essential for KRAS Q61K to be oncogenic. Without G60G silent mutation, KRAS undergoes alternative splicing and results in non-functional protein.

FIG. 31 shows splicing vulnerabilities in RAS Q61 mutant cancer. A hotspot of exonic splicing enhances (ESE) is around RAS Q61.

FIG. 32 shows currently FDA-approved antisense oligo nucleotides to modify splicing and the new strategy of using mutant-specific antisense oligos for the treatment of RAS Q61 cancers.

FIG. 33 shows efficacy of mutant specific antisense oligos in Q61 pan-cancer.

FIG. 34 shows examples of cancer types in KRAS/NRAS/HRAS Q61X (any kind of Q61 mutation, such as Q61K/L/H/R) from 3 independent cohorts.

FIG. 35 shows data about screening antisense oligos. Panel a: schema depicting the design of the KRAS Q61L specific antisense oligos. Morpholinos were designed with 3nt differences. Panel b: skipping whole exon 3 in KRAS Q61L mutant lung H650 cell line. Specifically, PCR bands showing KRAS exon 3 skipping in KRAS Q61L mutant lung H650 cell lines treated with 9 kinds of morpholinos. Panel c: skipping whole exon 3 quantified by qPCR in KRAS Q61L mutant lung H650 cell line. Specifically, qPCR data showing expression level of KRAS exon 3 skipping and full length KRAS (probes were designed in junction of exon 3 and exon 4) in H650 cell line. Panel d: shows images of PCR products after agarose gel electrophoresis.

FIG. 36 shows a table of morpholinos and sequences of morpholinos for target variants in KRAS used in the screening.

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Nucleic Acids

Aspects of the invention are directed towards nucleic acids comprising a sequence that hybridizes to a target. In embodiments, the target comprises a precursor mRNA of a RAS family gene.

The RAS family includes the homologous proteins NRAS, HRAS, and KRAS (Parikh et al, Blood 108:1, 2006). The RAS family members encode 21-kD guanine nucleotide binding proteins that operate as molecular switches to regulate the transduction of physiological signals from the cell membrane to the nucleus (Mackenzie et al, Blood 93:6, 1999).

The RAS family members have been found in several kinds of cancer. HRAS mutations are associated with a variety of cancers (Young et al, Oncogene 24:40, 2005; Perkins et al, Mol. Brain Res. Ill, 2003; Garrett et al, Cancer Epidemiol. Biomarkers Prev. 2, 1993; Theodorescu et al, Proc. Natl Acad. Sci. USA 87, 1990; Weston et al, Environ Health Perspect. 105:4, 1997; Fujita et al., Cancer Research 48, 1988). KRAS and HRAS mutations are associated with a variety of cancers (Braun et al, Proc. Natl Acad. Sci. USA 101:2, 2004; Ji et al, J. Biol. Chem. 282: 19, 2007; Pao et al, PLOS Medicine 2: 1, 2005; Rosell et al, Clin. Cancer Res. 2, 1996; Ryan et al, Gut 52, 2003). In human tumors, activation of NRAS genes are a consequence of amino acid substitutions at positions 12, 13, or 61 of an NRAS gene (Carney et al, Proc. Natl Acad. Sci. USA 83, 1986; Janssen et al, Proc. Natl Acad. Sci. USA 84, 1987; Ugurel et al, PLOS ONE 2:2, 2007; Mackenzie et al, Blood 93:6, 1999). Mutations that result in constitutive activation of NRAS have been shown to occur in myeloid malignancies (Parikh et al, 2006; Janssen et al, 1987) and carcinomas (Reynolds et al, Proc. Natl Acad. Sci. USA 88, 1991).

“Oligonucleotide compounds” (can be used interchangeably with “nucleic acids”) of the invention can include oligonucleotides, e.g., antisense oligonucleotides (ASOs), splice switching oligonucleotides (SSOs), siRNA, shRNA, and the like as well as modified nucleotides discussed herein that are incorporated into the same. ASOs are single stranded nucleotide molecules that are complementary to a target nucleic acid sequence. Thus, embodiments herein comprise an oligonucleotide compound (such as a nucleic acid) comprising a sequence that hybridizes to a target.

The term “target” is used in a variety of different forms throughout this document and is defined by the context in which it is used. “Target mRNA”, for example, can refer to a messenger RNA to which a given oligonucleotide can be directed against. “Target sequence” and “target site”, for example, can refer to a sequence within the mRNA to which the sense strand of an oligonucleotide shows varying degrees of homology and the antisense strand exhibits varying degrees of complementarity. The phrase “oligonucleotide target” can refer to the gene, mRNA, or protein against which an oligonucleotide is directed. In embodiments, the target nucleic acid encompasses DNA comprising a RAS family gene, RNA (including pre-mRNA and mRNA) transcribed from such DNA, and also cDNA derived from such RNA, as well as DNA or RNA sequence described herein that further encompasses noncoding sequence. In one embodiment, the target sequence comprises a nucleic acid sequence encoding a RAS pre-mRNA (e.g., KRAS, NRAS, HRAS), where the nucleic acid sequence includes but is not limited to sense and/or antisense non-coding and/or coding sequences associated with a nucleic acid sequence encoding a subunit of the MMR system.

The genomic sequence for human KRAS is accessible in public databases and has GenBank Accession number [NG_007524.2] (nucleic acid) and [NP_203524.1 and NP_004976.2] (protein). The KRAS protein has 2 isoforms, specifically isoform 4A with exon 5, and isoform 4B without exon 5.

The genomic sequence for human NRAS is accessible in public databases and has GenBank Accession number [NG_007572.1] (nucleic acid) and [NP_002515.1] (protein).

The genomic sequence for human HRAS is accessible in public databases and has GenBank Accession number [NG_007666.1] (nucleic acid) and [NP_005334.1 and NP_789765.1] (protein).

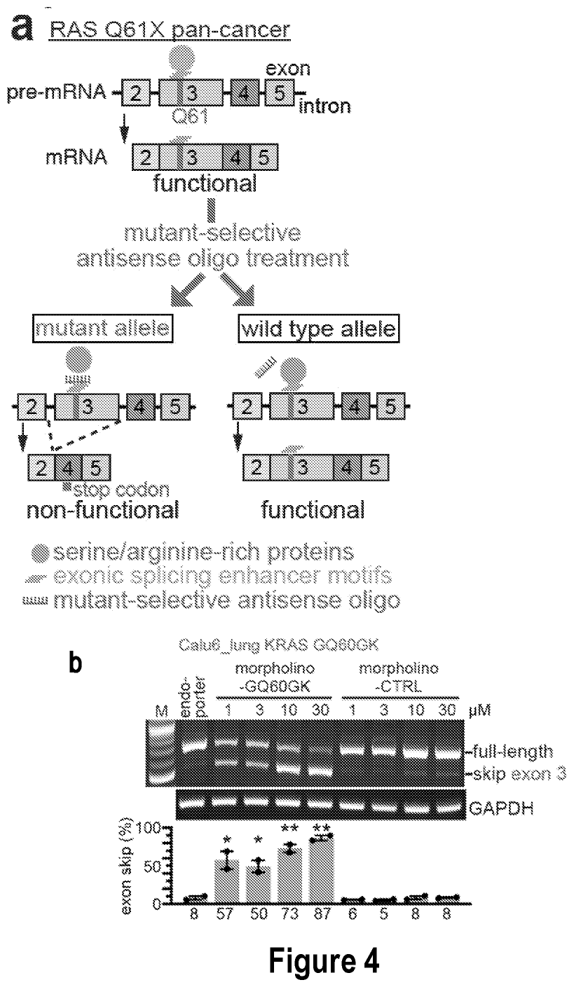

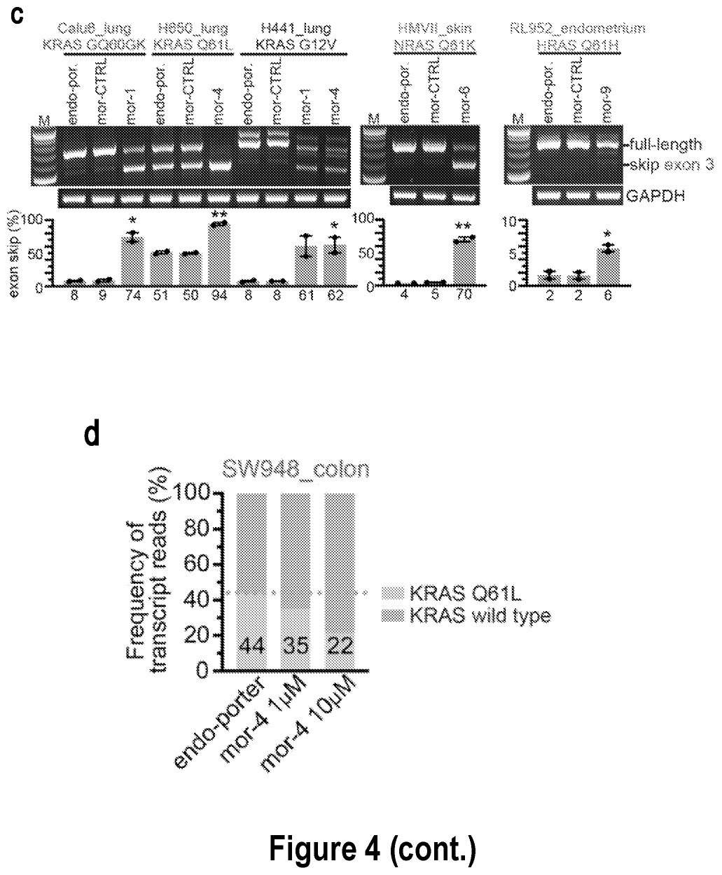

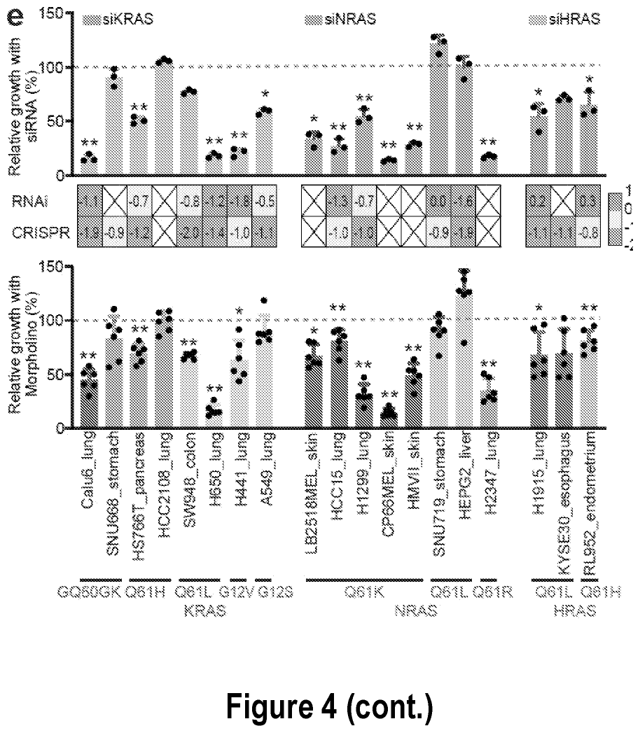

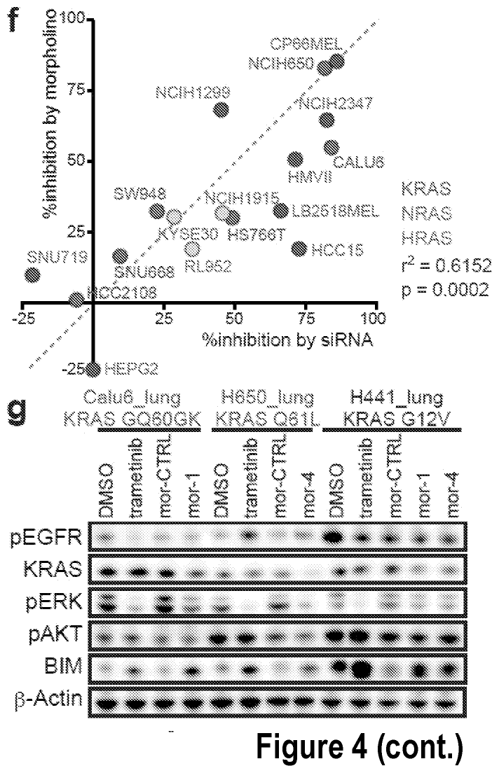

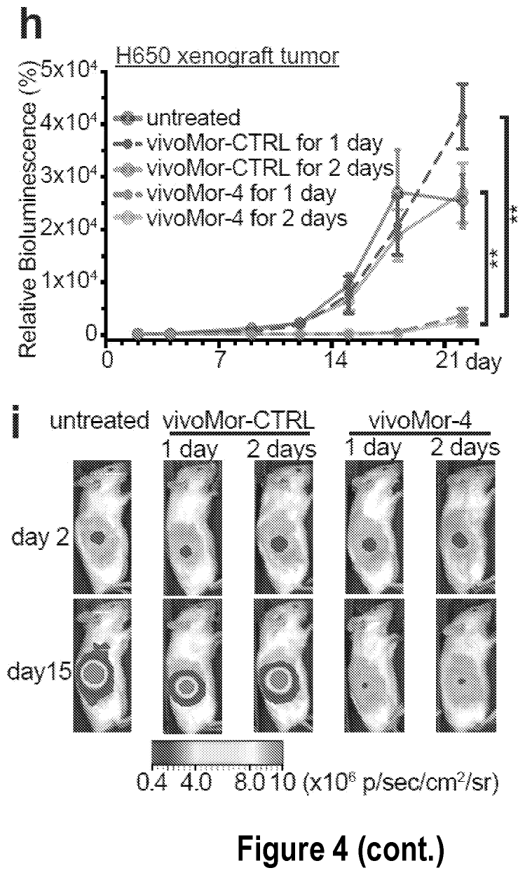

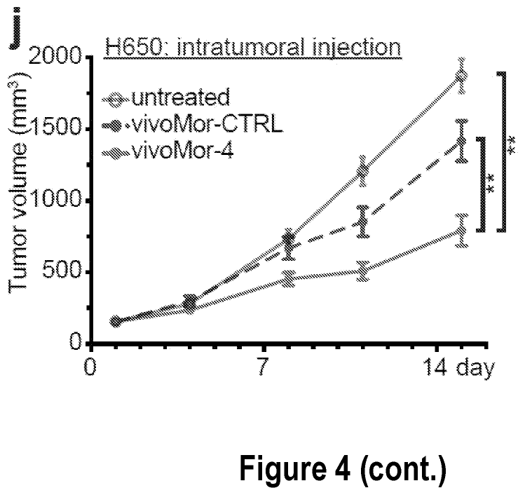

In embodiments, the target nucleic acid encompasses a precursor-mRNA comprising an exon splicing enhancer (ESE) binding motif or a putative exon splicing enhancer binding motif. Exon splicing enhancers are degenerate hexameric sequences found in exons which contribute to the splicing of the pre-mRNA transcribed from that gene. The presence or absence of exon splicing enhancers affects splicing. Thus, embodiments herein provide compositions and methods for skipping expression of a target exon of a gene in a cell, for example, wherein the target exon comprises a putative exon splicing enhancer sequence or is adjacent to a putative exon splicing enhancer sequence. Referring to FIG. 4, panel a, for example, the exon splicing enhancer motif can be located within or immediately adjacent to exon 3 of RAS.

KRAS exon 3
SEQ ID NO: [ ]
GATTCCTACAGGAAGCAAGTAGTAATTGATGGAGAAACCTGTCTCTTGGATATTCTCGACAC
AGCAGGTCAAGAGGAGTACAGTGCAATGAGGGACCAGTACATGAGGACTGGGGAGGGCTTTC
TTTGTGTATTTGCCATAAATAATACTAAATCATTTGAAGATATTCACCATTATAG
NRAS exon 3
SEQ ID NO: [ ]
GATTCTTACAGAAAACAAGTGGTTATAGATGGTGAAACCTGTTTGTTGGACATACTGGATAC
AGCTGGACAAGAAGAGTACAGTGCCATGAGAGACCAATACATGAGGACAGGCGAAGGCTTCC
TCTGTGTATTTGCCATCAATAATAGCAAGTCATTTGCGGATATTAACCTCTACAG
HRAS exon 3
SEQ ID NO: [ ]
GATTCCTACCGGAAGCAGGTGGTCATTGATGGGGAGACGTGCCTGTTGGACATCCTGGATAC
CGCCGGCCAGGAGGAGTACAGCGCCATGCGGGACCAGTACATGCGCACCGGGGAGGGCTTCC
TGTGTGTGTTTGCCATCAACAACACCAAGTCTTTTGAGGACATCCACCAGTACAG

In embodiments, the exon splicing enhancer (ESE) binding motif comprises codons 52 to 70 of the pre-mRNA or a portion thereof. For example, the exon splicing enhancer (ESE) binding motif comprises codons 55 to 65 of pre-mRNA of KRAS, or a portion thereof. For example, the exon splicing enhancer (ESE) binding motif comprises codons 55 to 66 of pre-mRNA of NRAS, or a portion thereof. For example, the exon splicing enhancer (ESE) binding motif comprises codons 58 to 67 pre-mRNA of HRAS, or a portion thereof.

Pre-mRNA of KRAS exon 3
SEQ ID NO:[ ]
GAUUCCUACAGGAAGCAAGUAGUAAUUGAUGGAGAAACCUGUCUCUUGGAUAUUCUCGACAC
AGCAGGUCAAGAGGAGUACAGUGCAAUGAGGGACCAGUACAUGAGGACUGGGGAGGGCUUUC
UUUGUGUAUUUGCCAUAAAUAAUACUAAAUCAUUUGAAGAUAUUCACCAUUAUAG
Pre-mRNA of NRAS exon 3
SEQ ID NO: [ ]
GAUUCUUACAGAAAACAAGUGGUUAUAGAUGGUGAAACCUGUUUGUUGGACAUACUGGAUAC
AGCUGGACAAGAAGAGUACAGUGCCAUGAGAGACCAAUACAUGAGGACAGGCGAAGGCUUCC
UCUGUGUAUUUGCCAUCAAUAAUAGCAAGUCAUUUGCGGAUAUUAACCUCUACAG
Pre-mRNA of HRAS exon 3
SEQ ID NO: [ ]
GAUUCCUACCGGAAGCAGGUGGUCAUUGAUGGGGAGACGUGCCUGUUGGACAUCCUGGAUAC
CGCCGGCCAGGAGGAGUACAGCGCCAUGCGGGACCAGUACAUGCGCACCGGGGAGGGCUUCC
UGUGUGUGUUUGCCAUCAACAACACCAAGUCUUUUGAGGACAUCCACCAGUACAG

In embodiments, the target comprises a precursor-mRNA (pre-mRNA) of a RAS family gene, wherein the precursor-mRNA comprises an exon splicing enhancer (ESE) binding motif comprising at least one mutation. As described herein, the significance of silent mutations and splicing vulnerabilities in Q61 mutant RAS cancers has been discovered. Q61X mutation is located within exon 3 of RAS, and is within and/or immediately adjacent to an exon splicing enhancer motif. Models with KRAS Q61K underwent alternative splicing due to the similarity to conserved motif of splicing donor site and resulted in causing early stop codon (and as such a non-functional protein), whereas KRAS Q61K+silent mutation (G60G+Q61K) prevented from alternative splicing, resulting in a functional protein. Thus, the target nucleic acid as described herein can be an ESE binding motif comprising at least one mutation as compared to the wild-type sequence, at least two mutations, at least three mutations, or four or more mutations. For example, the mutation can be Q61K. In another example, the mutation can be G60G+Q61K.

In embodiments the target nucleic acid can comprise at least one mutation within codon 61. For example, the at least one mutation comprises Q61H, Q61L, Q61R, or Q61K. In embodiments, the target nucleic acid can comprise at least one mutation within codon 60. In embodiments, the target nucleic acid can comprise a mutation within codon 60 and a mutation within codon 61. In embodiments, the mutations comprise G60X, Q61X, or a combination thereof. For example, the mutations comprise GQ60GK.

Thus, oligonucleotide compositions and/or nucleic acids as described herein can modulate splicing of a pre-mRNA, such as a RAS pre-mRNA. The nucleic acid can promote splicing of the pre-mRNA, or the nucleic acid can inhibit splicing of the pre-mRNA. In embodiments, the oligonucleotide composition and/or nucleic acid promotes alternative splicing which excludes exon 3 or a portion thereof.

Hybridization can involve hydrogen bonding, which can be Watson-Crick, Hoogsteen or reversed Hoogsteen hydrogen bonding, between complementary nucleoside or nucleotide bases. For example, adenine and thymine are complementary nucleotides which pair through the formation of hydrogen bonds. Hybridization can occur under varying circumstances. Complementary, as is understood by the skilled artisan, can refer to the capacity for precise pairing between two nucleotides. For example, an oligonucleotide and the DNA or RNA are complementary to each other when a sufficient number of corresponding positions in each molecule are occupied by nucleotides which can hydrogen bond with each other. The sequence of an oligonucleotide does not need to be 100% complementary to that of its target nucleic acid to be specifically hybridizable. For example, an oligonucleotide can hybridize over one or more segments such that intervening or adjacent segments are not involved in the hybridization event (such as, e.g., a loop structure, mismatch or hairpin structure). Thus, “specifically hybridizable” and “complementary” are used to indicate a sufficient degree of complementarity or precise pairing where stable and specific binding occurs between the oligonucleotide and its target nucleic acid (e.g., the DNA or RNA target).

In one embodiment, the specific hybridization of an oligonucleotide compound with its target nucleic acid interferes with the normal function of the nucleic acid. In one embodiment, an oligonucleotide compound is specifically hybridizable when binding of the oligonucleotide to the target nucleic acid interferes with the normal function of DNA, the normal function of RNA, or the normal function and/or expression of the product encoded by the target nucleic acid, causing a modulation of function and/or activity. In one embodiment, an oligonucleotide compound can cause an exon to be spliced from RAS pre-mRNA, such as exon 3. See, for example, FIG. 4, panel a. When exon 3 is spliced, such as from a mutant RAS pre-mRNA, for example, a non-functional protein is produced.

The DNA functions to be interfered include, for example, replication and transcription. The RNA functions to be interfered include, for example, translocation of the RNA to the site of protein translation, translation of protein from the RNA, splicing of the RNA to yield one or more mRNA species, and/or catalytic activity, which can be engaged in or facilitated by the RNA. The overall effect of such interference with target nucleic acid function is modulation of the expression of an encoded product or oligonucleotides. In one embodiment, the modulation is a decrease or loss of the activity of the encoded product. In one embodiment, the modulation is a decrease or loss of expression of the encoded product. In one embodiment, the modulation is the alternative splicing of the encoded mRNA.

The oligonucleotide compounds described herein comprise about 70%, or about 75%, or about 80%, or about 85%, or about 90%, or about 91%, or about 92%, or about 93%, or about 94%, or about 95%, or about 96%, or about 97%, or about 98%, or about 99% sequence complementarity to a target region within the target nucleic acid sequence to which the oligonucleotide compound is targeted. For example, an oligonucleotide in which 18 of 20 nucleotides of the oligonucleotide compound are complementary to a target region, and can therefore specifically hybridize, can represent 90 percent complementarity.

In one embodiment, homology between an oligonucleotide and its target nucleic acid sequence is from about 50% to about 60%. In some embodiments, the homology is from about 60% to about 70%. In some embodiments, the homology is from about 70% to about 80%. In some embodiments, the homology is from about 80% to about 85%. In some embodiments, the homology is from about 85% to about 90%. In some embodiments, the homology is about 90%, about 91%, about 92%, about 93%, about 94%, about 95%, about 96%, about 97%, about 98%, about 99%, or 100%.

In one embodiment, sequence identity between an oligonucleotide and its target nucleic acid sequence is from about 50% to about 60%. In further embodiments, the homology is from about 60% to about 70%. In further embodiments, the homology is from about 70% to about 80%. In further embodiments, the homology is from about 80% to about 85%. In further embodiments, the homology is from about 85% to about 90%. In further embodiments, the homology is about 90%, about 91%, about 92%, about 93%, about 94%, about 95%, about 96%, about 97%, about 98%, about 99%, or 100%.

In embodiments, the sequence is at least partially complementary to, partially complementary to, or fully complementary to the target. For example, in one embodiment, complementarity between an oligonucleotide and its target nucleic acid sequence is from about 50% to about 60%. In another embodiment, the homology is from about 60% to about 70%. In another embodiment, the homology is from about 70% to about 80%. In another embodiment, the homology is from about 80% to about 85%. In another embodiment, the homology is from about 85% to about 90%. In another embodiment, the homology is about 90%, about 91%, about 92%, about 93%, about 94%, about 95%, about 96%, about 97%, about 98%, about 99%, or 100%.

“Perfectly” or “fully” complementary nucleic acid molecules means those in which a certain number of nucleotides of a first nucleic acid molecule hydrogen bond (anneal) with the same number of residues in a second nucleic acid molecule to form a contiguous double-stranded region. For example, two or more fully complementary nucleic acid molecule strands can have the same number of nucleotides (i.e., have the same length and form one double-stranded region, with or without an overhang) or have a different number of nucleotides (e.g., one strand can be shorter than but fully contained within another strand or one strand can overhang the other strand).

“Partially complementary” means those nucleic acid molecules having a substantially complementary sequence to another nucleic acid but that differs from the other nucleic acid by at least one or more nucleotides. In certain embodiments, a partially complementary nucleic acid specifically excludes a nucleic acid containing a sequence that is exactly complementary, that is, a complementary sequence that has 100% complementarity.

In embodiments, the nucleic acid molecule is complementary or partially complementary to the mutant RAS pre-mRNA, but is not complementary to the wild-type RAS pre-mRNA. See, for example, FIG. 4, panel a.

In embodiments, the target nucleic acid can comprise a sequence of:

(SEQ ID NO: [ ])
5′-CUC UUG GAU AUU CUC GAC ACA GCA GGX₁ X₂X₃X₄
GAG GAG UAC AGU GCA AUG AGG GAC CAG-3′.

X₁-X₄ correspond to one or more mutations relative to wildtype KRAS. For example, X₁ is G, A, U, or C; X₂ is G, A, U, or C; X₃ is G, A, U, or C; X₄ is G, U, A, or C; or any combination thereof. For example, the target nucleic acid can comprise a sequence of:

(SEQ ID NO: [ ])
5′-CUC UUG GAU AUU CUC GAC
ACA GCA GGU CUA GAG GAG UAC
AGU GCA AUG AGG GAC CAG-3′,
(SEQ ID NO: [ ])
5′-CUC UUG GAU AUU CUC GAC
ACA GCA GGU CAC GAG GAG UAC
AGU GCA AUG AGG GAC CAG-3′,
(SEQ ID NO: [ ])
5′-CUC UUG GAU AUU CUC GAC
ACA GCA GGG AAA GAG GAG UAC
AGU GCA AUG AGG GAC CAG-3′,
and
(SEQ ID NO: [ ])
5′-CUC UUG GAU AUU CUC GAC
ACA GCA GGC AAA GAG GAG UAC
AGU GCA AUG AGG GAC CAG-3′,

or a sequence at least 90% identical thereto.

In embodiments, the target nucleic acid can comprise a sequence of:

(SEQ ID NO: [ ])
5′-UUG UUG GAC AUA CUG GAU
ACA GCU GGA X₁X₂A GAA GAG UAC
AGU GCC AUG AGA GAC CAA-3′.

X₁-X₂ correspond to one or more mutations relative to wildtype NRAS. For example, X₁ is G, U, A, or C; X₂ is G, U, A, or C; or any combination thereof. For example, the target nucleic acid can comprise a sequence of:

(SEQ ID NO: [ ])
5′-UUG UUG GAC AUA CUG GAU
ACA GCU GGA CGA GAA GAG UAC
AGU GCC AUG AGA GAC CAA-3′,
(SEQ ID NO: [ ])
5′-UUG UUG GAC AUA CUG GAU
ACA GCU GGA AAA GAA GAG UAC
AGU GCC AUG AGA GAC CAA-3′,
(SEQ ID NO: [ ])
5′-UUG UUG GAC AUA CUG GAU
ACA GCU GGA CUA GAA GAG UAC
AGU GCC AUG AGA GAC CAA-3′,

or a sequence at least 90% identical thereto.

In embodiments, the target nucleic acid can comprise a sequence of:

(SEQ ID NO: [ ])
5′-CUG UUG GAC AUC CUG GAU
ACC GCC GGC CX1X2 GAG GAG UAC
AGC GCC AUG CGG GAC CAG-3′.

X₁-X₂ correspond to one or more mutations relative to wildtype HRAS. For example, X₁ is G, U, A, or C; X₂ is G, U, A, or C; or any combination thereof. For example, the target nucleic acid can comprise a sequence of:

(SEQ ID NO: [ ])
5′-CUG UUG GAC AUC CUG GAU
ACC GCC GGC CUG GAG GAG UAC
AGC GCC AUG CGG GAC CAG-3′
(SEQ ID NO: [ ])
5′-CUG UUG GAC AUC CUG GAU
ACC GCC GGC CAU GAG GAG UAC
AGC GCC AUG CGG GAC CAG-3′

or a sequence at least 90% identical thereto.

In one embodiment, the oligonucleotides (i.e., nucleic acid molecules) can be specific for (i.e., hybridize to) a pre-mRNA of a RAS family gene (e.g., KRAS, NRAS, HRAS), which includes, without limitation, coding and non-coding regions. In one embodiment, the oligonucleotide is an antisense RNA molecule. In one embodiment, the oligonucleotide is an antisense DNA molecule. In one embodiment, an oligonucleotide targets a natural antisense sequence (natural antisense to the coding and non-coding regions) of a RAS family gene (e.g., KRAS, NRAS, HRAS). In one embodiment, the oligonucleotide is an antisense RNA molecule. In one embodiment, the oligonucleotide is an antisense DNA molecule.

The oligonucleotide compounds discussed herein can also include variants in which a different base is present at one or more of the nucleotide positions in the oligonucleotide compound. For example, if the first nucleotide is an adenine, variants can be produced which contain thymidine, guanosine, cytidine or other natural or non-natural nucleotides at that position. The base substitution can be done at any of the positions of the oligonucleotide. The oligonucleotide compounds can then be tested using methods described herein to determine the oligonucleotide compound's ability to inhibit expression and/or function of a target nucleic acid, such as a member of RAS (e.g., KRAS, HRAS, NRAS).

In one embodiment, the oligonucleotides comprise isolated oligonucleotides. As used herein, the term “isolated” can refer to referenced material (e.g., nucleic acid molecules of the disclosure herein) that are removed from its original environment, such as being separated from some or all of the co-existing materials in a natural environment (e.g., a natural environment can be a cell).

In embodiments, the oligonucleotide comprises a sequence according to

(SEQ ID NO: [ ])
5′-GTACTCCTCX₁X₂X₃X₄CCTGCTGTGTCG-3′,

or a sequence that is at least 90% identical thereto.

For example, X₁ is G or T; X₂ is A or T; X₃ is G or T; X₄ is G, C, or A; or any combination thereof. For example, the oligonucleotide comprises a sequence of

M1
(SEQ ID NO: [ ])
5′-GTACTCCTCTTTGCCTGCTGTGTCG-3′,
M2
(SEQ ID NO: [ ])
5′-GTACTCCTCTTTCCCTGCTGTGTCG-3′,
M3
(SEQ ID NO: [ ])
5′-GTACTCCTCGTGACCTGCTGTGTCG-3′,
M4
(SEQ ID NO: [ ])
5′-GTACTCCTCTAGACCTGCTGTGTCG-3′,

or a sequence that is at least 90% identical thereto.

In embodiments, the oligonucleotic comprises a sequence according to:

[SEQ ID NO: [ ]]
5′-CTTX₁X₂CCTGCTGTGTCGAGA-3′,

or a sequence that is at least 90% identical thereto.

For example, X₁ is G or T; X₂ is G, C or A; or any combination thereof.

For example, the oligonucleotide comprises a sequence of

1-1
SEQ ID NO: [ ]]
5′-CTTTGCCTGCTGTGTCGAGA-3′,
1-2
[SEQ ID NO: [ ]]
5′-CCTCTTTGCCTGCTGTGTCG-3′,
1-3
[SEQ ID NO: [ ]]
5′-ACTCCTCTTTGCCTGCTGTG-3′,
1-4
[SEQ ID NO: [ ]]
5′-TGTACTCCTCTTTGCCTGCT-3′,
1-5
[SEQ ID NO: [ ]]
5′-CACTGTACTCCTCTTTGCCT-3′,
or
1-6
[SEQ ID NO: [ ]]
5′-TTGCACTGTACTCCTCTTTG-3′.

In embodiments, the oligonucleotide comprises a sequence of

5′-X₁X₂X₃X₄-3′,

wherein the sequence further comprises 5′ flanking nucleotides, 3′ flanking nucleotides, or both 5′ flanking nucleotides and 3′ flanking nucleotides. In this context, the term “flanking nucleotides” can refers to the nucleotides adjacent to the 5′-X₁X₂X₃X₄-3′ sequence. In certain embodiments, the flanking nucleotides comprise 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In embodiments, the sequence comprises flanking nucleotides on its 5′ end, 3′ end, or both. In embodiments, the flanking sequences are a length suitable for hybridization to a target nucleic acid.

In embodiments, the flanking nucleotides can comprise between 0 and 50 nucleotides, such as between 0 and 40 nucleotides, such as between 0 and 30 nucleotides, such as between 0 and 20 nucleotides, such as between 0 and 10 nucleotides. In embodiments, the flanking nucleotides can comprise a sequence that is complementary to or partially complementary to a RAS pre-mRNA.

For example, X₁ is G or T; X₂ is A or T; X₃ is G or T; X₄ is G, C, or A; or any combination thereof. For example, the oligonucleotide comprises a sequence of

mor-4-1
(SEQ ID NO: [ ])
5′-TAGACCTGCTGTGTCGAGAATATCC-3′
mor-4-2
(SEQ ID NO: [ ])
5′-CTCTAGACCTGCTGTGTCGAGAATA-3′
mor-4-3
(SEQ ID NO: [ ])
5′-CTCCTCTAGACCTGCTGTGTCGAGA-3′
mor-4-4
(SEQ ID NO: [ ])
5′-GTACTCCTCTAGACCTGCTGTGTCG-3′
mor-4-5
(SEQ ID NO: [ ])
5′-ACTGTACTCCTCTAGACCTGCTGTG-3′
mor-4-7
(SEQ ID NO: [ ])
5′-CATTGCACTGTACTCCTCTAGACCT-3′
mor-4-8
(SEQ ID NO: [ ])
5′-CCTCATTGCACTGTACTCCTCTAGA-3′

In embodiments, the oligonucleotide comprises a sequence of

(SEQ ID NO: [ ])
5′-CTCTTCTX₁X₂TCCAGCTGTATCCAGT-3′,

or a sequence that is at least 90% identical thereto.

For example, X₁ is C, T, or A; X₂ is T or G; or any combination thereof. For example, the oligonucleotide comprises a sequence of

(SEQ ID NO: [ ])
5′-CTCTTCTCGTCCAGCTGTATCCAGT-3′,
(SEQ ID NO: [ ])
5′-CTCTTCTTTTCCAGCTGTATCCAGT-3′,
(SEQ ID NO: [ ])
5′-CTCTTCTAGTCCAGCTGTATCCAGT-3′,

or a sequence that is at least 90% identical thereto.

In embodiments, the oligonucleotide comprises a sequence of:

(SEQ ID NO: [ ])
5′-TGGCGCTGTACTCCTCX_X2GGCCGG-3′,

or a sequence that is at least 90% identical thereto.

For example, X₁ is A or C; X₂ is T or A; or any combination thereof. For example, the oligonucleotide comprises a sequence of:

(SEQ ID NO: [ ])
5′-TGGCGCTGTACTCCTCCAGGCCGG-3′,
(SEQ ID NO: [ ])
5′-TGGCGCTGTACTCCTCATGGCCGG-3′,

or a sequence that is at least 90% identical thereto.

According to the invention, oligonucleotide compounds can comprise at least one region where the oligonucleotide is modified in order to exhibit one or more properties described herein. The properties of the oligonucleotide include, but are not limited, for example, to increased resistance to nuclease degradation, increased cellular uptake, and/or increased binding affinity for the target nucleic acid. Modified oligonucleotides can include, for example, synthetic nucleotides having modified base moieties and/or modified sugar moieties (see e.g., described by Schcit, Nucleotide Analogs, John Wiley, New York, 1980; Freier & Altmann, (1997) Nucl. Acid. Res., 25(22), 4429-4443, Toulme, J. J., (2001) Nature Biotechnology 19:17-18; Manoharan M., (1999) Biochemica et Biophysica Acta 1489:117-139; Freier S. M., (1997) Nucleic Acid Research, 25:4429-4443, Uhlman, E., (2000) Drug Discovery & Development, 3: 203-213, Herdewin P., (2000) Antisense & Nucleic Acid Drug Dev., 10:297-310); or 2′-O, 3′-C-linked [3.2.0]bicycloarabinonucleosides. Such modified nucleotides include synthetic nucleotides designed to enhance binding properties, e.g., duplex or triplex stability, specificity, or the like.

An oligonucleotide compound, whether DNA or RNA with modified nucleotides, DNA or RNA with substituted nucleotides, and the like, can specifically hybridize when binding of the oligonucleotide compound to the target nucleic acid (e.g., a DNA or RNA molecule) interferes with the normal function of the target DNA or RNA. Further modifications can include conjugate groups attached to one of the termini of an oligonucleotide compound or to selected nucleotide positions of an oligonucleotide compound, conjugate group(s) added to various positions on the sugar ring, or conjugate group(s) added to one of the internucleotide linkages. In one embodiment, the interference can cause a loss of utility of the target nucleic acid, and there is a sufficient degree of complementarity to avoid non-specific binding of the oligonucleotide compound to non-target nucleic acid sequences under conditions in which specific binding is need. Conditions in which specific binding is needed include, but are not limited to, physiological conditions in in vivo assays or in therapeutic treatment, or conditions in which the in vitro assays are performed.

ASOs comprise a grouping of antisense compounds which include but are not limited to siRNA, ribozymes, external guide sequence (EGS) oligonucleotides, single- or double-stranded RNA interference (RNAi), and other oligonucleotides that hybridize to at least a portion of the target nucleic acid sequences and modulate its function. The antisense compounds can be single-stranded, double-stranded, circular or hairpin and can comprise structural elements such as mismatches or loops. Antisense compounds are routinely prepared linearly but one of ordinary skill in the art can prepare antisense compounds to be joined or otherwise prepared to be circular and/or branched.

In one embodiment, oligonucleotide compounds directed to a nucleic acid sequence of a RAS pre-mRNA can comprise one or more modified nucleotides. In one embodiment, oligonucleotide compounds can comprise shorter or longer fragment lengths (e.g., 15-, 16-, 17-, 18-, 19-20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49-, or 50-mers). In one embodiment, oligonucleotide compounds can comprise modified bonds or internucleotide linkages. Non-limiting examples of modified bonds or internucleotide linkages include phosphorothioate, phosphorodithioate, and the like. In one embodiment, the oligonucleotide compounds can comprise a phosphorus derivative. In one embodiment, the phosphorus derivative (or modified phosphate group) can be attached to the sugar or sugar analog moiety in the modified oligonucleotides of the invention. Non-limiting examples of a phosphorus derivative (or a modified phosphate group) include a monophosphate, diphosphate, triphosphate, alkylphosphate, alkanephosphate, phosphorothioate and the like. The preparation of the exemplary phosphorus derivatives (or modified phosphate groups), and their incorporation into nucleotides (e.g., those comprising an oligonucleotide compound of the invention), is well-known in the art. In embodiments, the modified nucleic acid comprises a sugar modification, a backbone modification, a base modification, an unnatural base pair, conjugation to a cell penetrating peptide, or any combination thereof. In embodiments, the modification comprises phosphorothioate (PS)+2′-O-Methyoxyethyl (2′MOE). In embodiments, the modified nucleic acid is a morpholino, a locked nucleic acid (LNA), amido-bridged nucleic acid (AmNA), or peptide nucleic acid (PNA).

A number of nucleotide modifications have been shown to make the oligonucleotide into which they are incorporated more resistant to nuclease digestion than the native oligodeoxynucleotide. Oligonucleotides that have been modified to enhance their nuclease resistance survive intact for a longer time than unmodified oligonucleotides. As discussed herein, embodiments of the invention encompass modified oligonucleotides, such as modified ASOs directed to a RAS pre-mRNA. Modified oligonucleotides can comprise 2′-O-methyl modified oligoribonucleotides, which render the antisense oligonucleotide resistant to RNase H degradation. In one embodiment, modified oligonucleotides comprise a phosphorothioate backbone. For example, the phosphorothioate backbone increases the stability of an oligonucleotide compound against nucleases and enhances cellular uptake. In some embodiments, oligonucleotide compounds can comprise a full length phosphorodiamidate DNA. In some embodiments, oligonucleotide compounds can comprise one or nucleotides having a 2′O-methyl modification. In some embodiments, oligonucleotide compounds comprise one or more modifications discussed herein. Non-limiting examples of modified backbones include phosphorothioates, phosphinates, phosphorodithioates, phosphoramidates, phosphotriesters, aminoalkylphosphotriesters, methyl and other alkyl phosphonates (e.g., phosphonates comprising 3′ alkylene phosphonates), short chain alkyl or cycloalkyl intersugar linkages or short chain heteroatomic or heterocyclic intersugar linkages. In one embodiment, oligonucleotide compounds directed to a nucleic acid sequence of a RAS pre-mRNA comprise phosphorothioate backbones.

In one embodiment, a modified oligonucleotide compound comprises at least one nucleotide modified at the 2′ position of the sugar. In some embodiments, the nucleotide having a modification at the 2′ position of the sugar comprises a 2′-O-alkyl, 2′-O-alkyl-O-alkyl, or 2′-fluoro-modified nucleotide. In some embodiments, RNA modifications include 2′-fluoro, 2′-amino and 2′-O-methyl modifications on the ribose of pyrimidines.

As discussed herein, oligonucleotide compounds can comprise additional modifications such as morpholino phosphorodiamidate DNA, locked nucleic acids (LNA), and ethylene bridged nucleic acids. These modifications can render the oligonucleotide compounds RNase H and nuclease resistant as well as can increase the affinity for the target RNA. In one embodiment, oligonucleotide compositions of the invention have morpholino backbone structures (e.g., as disclosed by Summerton and Weller, in U.S. Pat. No. 5,034,506, which is hereby incorporated by reference in its entirety). Morpholinos, for example, are commercially available through Gene Tools, LLC, Philomath Oreg.; http://www.gene-tools.com/).

For example, the morpholino backbone of oligonucleotide analogues can make them resistant to nucleases and proteases so that they are long-lived in the cell.

For example, the morpholino can be a peptide-conjugated morpholino (e.g. as described in Boisguérin et al. Advanced Drug Delivery Review 87 (2015) 52-67 and Klien et al. The Journal of Clinical Investigation, 2019, 129(11):4739-4744, which are hereby incorporated by reference in their entirety). For example, the peptide-conjugated morpholino can be a “cell penetrating peptide-conjugated morpholino”. In embodiments, the cell penetrating peptide-conjugated morpholino can have increased delivery in the body. Cell penetrating peptide-conjugated morpholino will be known to the skilled artisan. For example, see Klein, Arnaud F., et al. “Peptide-conjugated oligonucleotides evoke long-lasting myotonic dystrophy correction in patient-derived cells and mice.” The Journal of clinical investigation 129.11 (2019): 4739-4744, and Boisguérin, Prisca, et al. “Delivery of therapeutic oligonucleotides with cell penetrating peptides.” Advanced drug delivery reviews 87 (2015): 52-67.

A number of nucleotide modifications incorporated into an oligonucleotide (e.g., resulting in an oligonucleotide analog), makes the oligonucleotide useful for steric blocking applications. For example, negatively charged oligonucleotide analogues, such as oligodeoxynucleotide phosphorothioate (DNA-PS), 2′-O-methylphosphorothioate (OMe-PS), 2′-O-methoxyethyl (MOE), 2′-deoxy-2′-fluoronucleotides (2′-F), locked nucleic acids (LNA; also referred to as bridged nucleic acids (BNA)), ethylene-bridged nucleic acids (ENA), tricycloDNA analogue (TcDNA), and 2′-O-[2-(N-methylcarbamoyl)ethyl]uridine (MCE), as disclosed in Järver et al. (2014) Nuc. Acid Therap., 24(1):37-47 (incorporated by reference in its entirety), can be used to induce exon skipping:

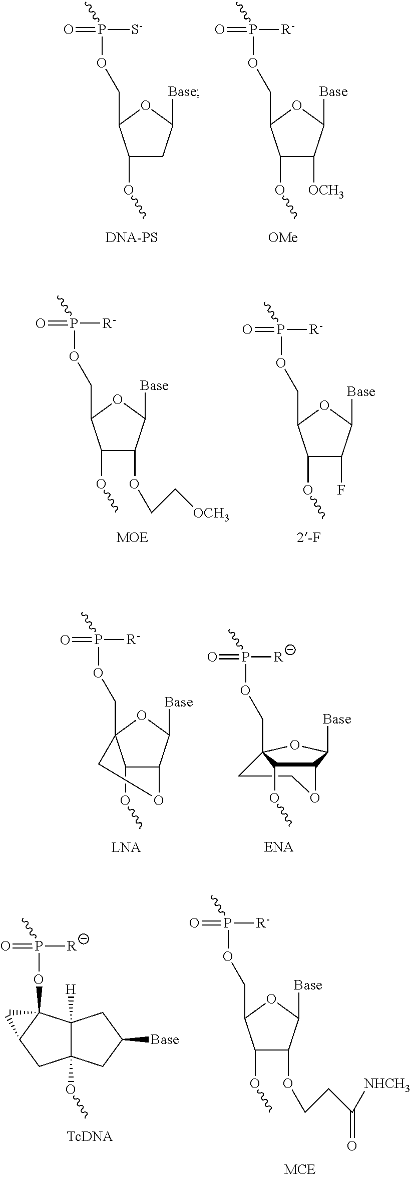

where R can be O or S in the negatively charged oligonucleotide analogues.

In one embodiment, the oligonucleotide compounds disclosed herein comprise one or more substitutions or modifications. In one embodiment, the oligonucleotide compounds are substituted with at least one locked nucleic acid (LNA). In one embodiment, the oligonucleotide compounds are substituted with at least one phosphorothioate (PS). In one embodiment, the oligonucleotide compounds are substituted with at least one 2′-O-methylphosphorothioate (OMe-PS). In one embodiment, the oligonucleotide compounds are substituted with at least one 2′-O-methoxyethyl (MOE). In one embodiment, the oligonucleotide compounds are substituted with at least one 2′-deoxy-2′-fluoronucleotide (2′-F). In one embodiment, the oligonucleotide compounds are substituted with at least one ethylene-bridged nucleic acid (ENA). In one embodiment, the oligonucleotide compounds are substituted with at least one tricycloDNA analogue (TcDNA). In one embodiment, the oligonucleotide compounds are substituted with at least one 2′-O-[2-(N-methylcarbamoyl)ethyl]uridine (MCE). In one embodiment, the oligonucleotide compounds are substituted with at least one oligodeoxynucleotide phosphorothioate (DNA-PS), 2′-O-methylphosphorothioate (OMe-PS), 2′-O-methoxyethyl (MOE), 2′-deoxy-2′-fluoronucleotide (2′-F), locked nucleic acid (LNA), ethylene-bridged nucleic acid (ENA), tricycloDNA analogue (TcDNA), 2′-O-[2-(N-methylcarbamoyl)ethyl]uridine (MCE), or a combination thereof.

Charge-neutral peptide nucleic acids (PNA) and phosphorodiamidate morpholino oligonucleotides (PMO) are further examples of oligonucleotide analogues, as disclosed in Järver et al. (2014) Nuc. Acid Therap., 24(1):37-47 (incorporated by reference in its entirety), that can be used to induce exon skipping:

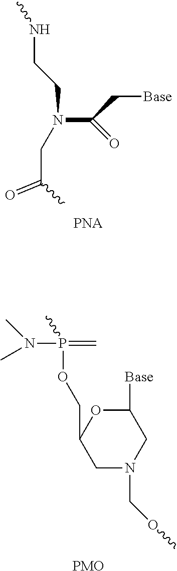

In one embodiment, the oligonucleotide compounds are substituted with at least one peptide nucleic acid (PNA). In one embodiment, the oligonucleotide compounds are substituted with at least one phosphorothioate (PS). In one embodiment, the oligonucleotide compounds are substituted with at least one peptide nucleic acid (PNA), phosphorothioate (PS), or a combination thereof.

Due to the uncharged backbone of the morpholino subunit, these oligonucleotide analogues can bind their complementary target RNA tightly. Morpholinos work by binding their complementary sequence and excluding binding by proteins or nucleic acids. In one embodiment, binding to an exon splicing enhancer binding motif can interfere with recognition of those sequences by the splicing machinery. Morpholinos have most often been used for protein knockdown experiments. A morpholino designed to bind the initiating AUG in an mRNA will block translation initiation by ribosomes. An advantage of morpholinos is the predictable way that they work in different species and different tissues since they are not dependent on accessory protein expression such as RISC, dicer, or RNaseH for activity.

In one embodiment, oligonucleotide compounds disclosed herein can bind to a selected target nucleic acid sequence to modulate splicing. In some embodiments, masking an exon splicing enhancer binding domain can modulate splicing. In one embodiment, an oligonucleotide compound as described herein can cause an exon to be retained; thus, when the exon is retained, for example, the mRNA can encode a functional protein. In one embodiment, the oligonucleotide compound is a modified oligonucleotide directed to a target nucleic acid sequence of RAS pre-mRNA. In another embodiment, the modified oligonucleotide compound directed to a target nucleic acid sequence of RAS pre-mRNA comprises at least one morpholino subunit.

In some embodiments, an oligonucleotide compound directed to a nucleic acid sequence of a RAS pre-mRNA is a modified oligonucleotide. According to the invention, a combination or “cocktail” of two or more oligonucleotide compounds can be provided that bind to a selected target nucleic acid in order to modulate splicing.

Target site(s) useful in the practice of the invention are those involved in mRNA splicing (such as splice donor sites, splice acceptor sites or exonic splicing enhancer elements). Splicing branch points and exon recognition sequences or splice enhancers are also potential target sites for modulation of mRNA splicing. In one embodiment, oligonucleotide compounds disclosed herein can bind to a selected target nucleic acid sequence to induce exon skipping. In some embodiments, masking a donor splice site can induce exon skipping. In some embodiments, masking an acceptor splice site can induce exon skipping. For example, owing to the nature of morpholino oligomers, one of ordinary skill in the art can identify sequences that will reliably bind splice junctions. As described in the examples herein, the efficacy of targeted morpholino SSOs can be quickly ascertained in tissue culture.

Another modification of the oligonucleotide compounds disclosed herein involves chemically linking one or more moieties or conjugates to the oligonucleotide, which enhance the activity, cellular distribution, or cellular uptake of the oligonucleotide. These moieties or conjugates can include conjugate groups covalently bound to functional groups such as primary or secondary hydroxyl groups. Non-limiting examples of moieties and conjugates include lipid moieties (such as a cholesterol moiety, a cholesteryl moiety, a thiocholesterol moeity), intercalators, reporter molecules, a palmityl moiety, or an octadecylamine or hexylamino-carbonyl-oxycholesterol moiety, a phospholipid, aliphatic chains (such as dodecandiol or undecyl residues), polyamine chains, polyamide chains, polyethylene glycol chains, polyether chains, cholic acid, and adamantane acetic acid. Examples of conjugate groups include, but are not limited to, cholesterols, lipids, phospholipids, biotin, phenazine, folate, phenanthridine, anthraquinone, acridine, fluoresceins, rhodamines, coumarins, and dyes.

Oligonucleotide compounds comprising lipophilic moieties, and methods for preparing such are known in the art, for example, as described in U.S. Pat. Nos. 5,138,045, 5,218,105 and 5,459,255, each of which is incorporated by reference in its entirety.

Representative United States patents that teach the preparation of oligonucleotide compound conjugates include, but are not limited to, U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is hereby incorporated by reference in its entirety. Representative conjugate groups are disclosed in U.S. Pat. Nos. 5,578,718; 6,153,737; 6,287,860; and 6,783,931, each of which are incorporated by reference in its entirety.

The oligonucleotide compounds can be conveniently and routinely made through the established technique of solid phase synthesis. Equipment useful for such syntheses can be obtained through several commercial vendors, including Applied Biosystems (Foster City, Calif.). Synthesis of the oligonucleotide compounds is well understood by one of ordinary skill in the art. It is also well known in the art to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides. Morpholinos, for example, are commercially available through Gene Tools, LLC, Philomath Oreg.; http://www.gene-tools.com/). For example, the oligonucleotide compounds of the invention (such as ASOs and SSOs) are synthesized in vitro and do not include antisense compositions of biological origin, or genetic vector constructs designed to direct the in vivo synthesis of oligonucleotide compounds.

In one embodiment, the oligonucleotide compounds (e.g., modified oligonucleotide compounds) bind to coding and/or non-coding regions of a target nucleic acid sequence of RAS pre-mRNA, and modulate the expression and/or function of the target molecule.

Target nucleic acid sequences of about 5-100 nucleotides in length, comprising a stretch of at least five (5) consecutive are suitable for targeting. Target nucleic acid sequences can include DNA or RNA sequences that comprise at least 5 consecutive nucleotides from the 5′-terminus of the gene encoding a RAS protein. Target nucleic acid sequences can include DNA or RNA sequences that comprise at least 5 consecutive nucleotides from the 3′-terminus of the gene encoding a RAS protein.

In one embodiment, the oligonucleotide compound binds to a sense or an antisense strand of a target nucleic acid sequence. The target nucleic acid sequences include coding as well as non-coding regions. The oligonucleotide compound can be from about 10 nucleotides in length up to about 50 nucleotides in length. In one embodiment, the oligonucleotide compounds of the invention are 10 to 50 nucleotides in length. In one embodiment, the oligonucleotide compounds are at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the oligonucleotides are 15 nucleotides in length. In some embodiments, the oligonucleotides are 20 nucleotides in length. In some embodiments, the oligonucleotides are 25 nucleotides in length. In some embodiments, the oligonucleotides are 20 nucleotides in length. In some embodiments, the oligonucleotides are 30 nucleotides in length.

### Treatments and Therapy for Diseases

As provided herein, any of the aspects or embodiments disclosed herein can be useful in treating RAS-associated diseases or disorders in a subject in need thereof, such as one or more hyperproliferative diseases or disorders, for example, leukemia, cutaneous melanoma, adenocarcinoma, squamous cell carcinoma, Philadelphia chromosome-negative myeloproliferative disorder, myelodysplastic syndrome, transitional cell carcinoma, ovarian cancer, brain tumors, breast cancer, bladder cancer, lung cancer, kidney tumors, urinary tract tumors, pancreatic carcinoma, and colorectal adenoma; as well as one or more angiogenic diseases or disorders. For example, the method can comprise administering to a subject a therapeutically effective amount of a nucleic acid molecule as described herein or a composition comprising the same.

In embodiments, the RAS-associated disease or disorder is cancer, such as a cancer comprising at least one mutation in KRAS, NRAS, or HRAS. For example, the cancer can comprise at least one mutation within codon 61 of RAS. For example, the at least one mutation comprises Q61H, Q61L, Q61R, or Q61K. In embodiments, the cancer can comprise at least one mutation within codon 60. In embodiments, the cancer can comprise a mutation within codon 60 and a mutation within codon 61. In embodiments, the mutations comprise G60X, Q61X, or a combination thereof. For example, the mutations comprise GQ60GK.

The terms “animal,” “subject,” and “patient” can be used interchangeably, and can refer to members of the animal kingdom including, but not limited to, mammals, animals (e.g., cats, dogs, horses, swine, etc.) and humans.

As used herein and as is well understood in the art, “treatment” is an approach for obtaining beneficial results, including clinical results. Beneficial clinical results can include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” can also refer to prolonging survival as compared to expected survival if not receiving treatment.

The term “in need thereof” can refer to the need for symptomatic or asymptomatic relief from a condition such as, for example, a RAS associated disease or condition, such as a cancer. The subject in need thereof can be undergoing treatment for conditions related to, for example, a cancer.

Aspects of the invention are also drawn to methods for modulating splicing of a RAS pre-mRNA in a subject. For example, the method comprises administering to a subject a therapeutically effective amount of a nucleic acid molecule as described herein or a composition comprising the same.

Aspects of the invention are further drawn to methods for modulating splicing of a RAS pre-mRNA in a tumor cell. For example, the method comprises contacting the tumor cell with a nucleic acid molecule as described herein in an amount effect to modulate splicing of the RAS pre-mRNA in the tumor cell. In embodiments, the splicing is modulated in tumor cells, such as those comprising a mutated RAS, but not in normal cells.

As used herein, “modulating splicing” can refer to changing the splicing pattern of an mRNA and includes promoting or inhibiting exon skipping, exon inclusion, intron inclusion, utilization of a nearby cryptic splice site, or generation of a new splice site. The alteration of the splicing pattern need not be 100%, i.e., promoting and inhibiting refer to increasing and decreasing the frequency that a splicing event occurs (or does not occur) relative to the frequency in the original pre-mRNA (without mutation or without compound treatment).

As described herein, modulating splicing can refer to modulating splicing of exon 3 of a RAS pre-mRNA.

The disclosure also comprises of a small molecule therapeutics (e.g., oligonucleotide compounds, naked or modified) useful for the treatment of RAS associated diseases or conditions, such as cancer. In one embodiment, an oligonucleotide compound (e.g., an antisense oligonucleotide) is administered to a subject to prevent or treat diseases or disorders associated with RAS. In one embodiment, an oligonucleotide compound is directed to a target nucleic acid sequence of a RAS pre-mRNA. In one embodiment, an effective amount of the oligonucleotide compound is administered to the subject. In some embodiments, the oligonucleotide compound is a modified oligonucleotide that is nuclease-resistant. In some embodiments, the oligonucleotide compound comprises a pharmaceutical composition administered to a subject in a pharmaceutically acceptable carrier. In some embodiments, the oligonucleotide compound (e.g., an antisense oligonucleotide modulates splicing) can serve as a therapeutic method for the treatment of various RAS associated diseases or conditions.

For therapeutics, a subject, for example, a human, suspected of having a disease or disorder (such as a RAS associated disease or condition), which can be treated by modulating the expression of a nucleic acid sequence of RAS is treated by administering an oligonucleotide compound (such as an ASO) in accordance with this invention. In one embodiment, a pharmaceutical composition comprising an oligonucleotide compound disclosed herein, such as a nuclease-resistant oligonucleotide 15 to 30 nucleotide bases in length targeted to a complementary nucleic acid sequence of a gene or gene product encoding a RAS protein, is administered to a subject. In one embodiment, the oligonucleotide hybridizes with and modulates the splicing of a RAS pre-mRNA by about 10%, about 20%, about 30%, about 40%, about 50%, about 60%, about 70%, about 75%, about 80%, about 85%, about 90%, about 95%, about 97%, about 98%, about 99%, or 100%, as compared to a normal control. In one embodiment, the oligonucleotide compound comprises at least one modification. In one embodiment, the oligonucleotide is 17 to 28 nucleotide bases in length. In one embodiment, the oligonucleotide is 18 to 25 nucleotide bases in length. In one embodiment, the oligonucleotide is 19 to 23 nucleotide bases in length.

In one embodiment, a pharmaceutical composition that is an oligonucleotide compound comprising an oligonucleotide complex can be administered.

The oligonucleotide compounds of the invention can be utilized in pharmaceutical compositions by adding an effective amount of a compound to a suitable pharmaceutically acceptable diluent or carrier. Use of the oligonucleotide compounds and methods of the invention can also be useful prophylactically.

An “effective amount”, “sufficient amount” or “therapeutically effective amount” as used herein is an amount of a composition that is sufficient to effect beneficial results, including clinical results. As such, the effective amount can be sufficient, for example, to reduce or ameliorate the severity and/or duration of an affliction or condition, or one or more symptoms thereof, prevent the advancement of conditions related to an affliction or condition, prevent the recurrence, development, or onset of one or more symptoms associated with an affliction or condition, or enhance or otherwise improve the prophylactic or therapeutic effect(s) of another therapy. An effective amount also includes the amount of the composition (e.g., the oligonucleotide compounds discussed herein) that avoids or substantially attenuates undesirable side effects.

The term “carrier” can refer to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. Other examples of suitable pharmaceutical carriers are described in Remington: The Science and Practice of Pharmacy, 21st Edition (University of the Sciences in Philadelphia, ed., Lippincott Williams & Wilkins 2005); and Handbook of Pharmaceutical Excipients, 7th Edition (Raymond Rowe et al., ed., Pharmaceutical Press 2012); each hereby incorporated by reference in its entirety.

The term “pharmaceutically acceptable salts” can refer to physiologically and pharmaceutically acceptable salts of the compounds of the invention: i.e., salts that retain the required biological activity of the parent compound and do not impart undesired toxicological effects thereto. A pharmaceutically acceptable carrier can comprise solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions. For oligonucleotide compounds, examples of pharmaceutically acceptable salts and their uses are further described in U.S. Pat. No. 6,287,860, which is hereby incorporated by reference in its entirety.

In one embodiment, modulation of splicing of a RAS pre-mRNA can be effected by administering one or more oligonucleotide compounds (e.g., ASOs or SSOs, naked or modified) to a subject in need thereof. In one embodiment, the prevention, amelioration, or treatment of a RAS-associated disease or condition that is related to abnormal expression, function, activity of a subunit of RAS protein compared to a normal control can also be effected by administering one or more oligonucleotide compounds (e.g., ASOs or SSOs, naked or modified) to a subject in need thereof.

Embodiments of the invention can be administered alone, or can be administered in a therapeutic cocktail or as a pharmaceutical composition. For example, a pharmaceutical composition can comprise embodiments of the invention, and a saline solution that includes a phosphate buffer. Embodiments of the invention can be administered using the means and doses described herein. Embodiments of the invention can be administered in combination with a suitable carrier. In one embodiment, the oligonucleotide compounds of the invention (e.g., ASOs and SSOs) encompass any pharmaceutically acceptable salts, esters, or salts of such esters, or any other compound which, upon administration to a subject, provides (directly or indirectly) the biologically active metabolite or residue thereof.

Within additional aspects of this disclosure, combination formulations and methods are provided comprising an effective amount of one or more oligonucleotides of the disclosure in combination with one or more secondary or adjunctive active agents that are formulated together or administered coordinately with the oligonucleotide of this disclosure to control a RAS-associated disease or condition as described herein. Useful adjunctive therapeutic agents in these combinatorial formulations and coordinate treatment methods include, for example, enzymatic nucleic acid molecules, allosteric nucleic acid molecules, antisense, decoy, or aptamer nucleic acid molecules, antibodies such as monoclonal antibodies, small molecules and other organic or inorganic compounds including metals, salts and ions, and other drugs and active agents indicated for treating a RAS-associated disease or condition, including chemotherapeutic agents used to treat cancer, steroids, non-steroidal anti-inflammatory drugs (NSAIDs), tyrosine kinase inhibitors, or the like. Exemplary chemotherapeutic agents include alkylating agents (e.g., cisplatin, oxaliplatin, carboplatin, busulfan, nitrosoureas, nitrogen mustards, uramustine, temozolomide), antimetabolites (e.g., aminopterin, methotrexate, mercaptopurine, fluorouracil, cytarabine), taxanes (e.g., paclitaxel, docetaxel), anthracyclines (e.g., doxorubicin, daunorubicin, epirubicin, idaruicin, mitoxantrone, valrubicin), bleomycin, mytomycin, actinomycin, hydroxyurea, topoisomerase inhibitors (e.g., camptothecin, topotecan, irinotecan, etoposide, teniposide), monoclonal antibodies (e.g., alemtuzumab, bevacizumab, cetuximab, gemtuzumab, panitumumab, rituximab, tositumomab, trastuzumab), vinca alkaloids (e.g., vincristine, vinblastine, vindesine, vinorelbine), cyclophosphamide, prednisone, leucovorin, oxaliplatin.

Some adjunctive therapies can be directed at targets that interact or associate with RAS or affect specific RAS biological activities. A variety of inhibitors of RAS have been described that can be suitably employed as adjunctive therapies including, but not limited to, small molecules and antibodies or fragments thereof.

Small molecule NRAS inhibitors include, for example, farnesyl transferase inhibitors, MEK1/2 inhibitors, and inhibitory RAS isoforms. Farnesyl transferase inhibitors, such as FTI-277 and R1 15777, interfere with the translocation of NRAS to the cell membrane (Ugurel et al., PLOS ONE 2:2, 2007; Lerner et al, Oncogene 15: 11, 1997; Ochiai et al, Blood 102:9, 2003). A recent study showed that MEK 1/2 inhibitors PD98059 and U0126 deactivated the NRAS-MEK-mitogen-activated protein kinase (MAPK) p44/42 pathway linked to ovine pulmonary adenocarcinoma (Maeda et al, J. Virol 79:1, 2005). Dominant inhibitory mutants have been created for each of the three RAS isoforms that substitute serine 17 with aspargine (Matallanas et al, J. Biol. Chem. 278:1, 2003). Two of the three RAS isoforms, NRAS N17 and HRAS N17, were shown to inhibit wildtype NRAS (Matallanas et al, J. Biol Chem. 278:1, 2003).

Small molecule HRAS inhibitors include, for example, farnesyl transferase inhibitors, S-Trans, Trans-farnesylthiosalicylic acid, and diallyl disulfide (DADS). Some known farnesyl transferase inhibitors include FTI-277 and R1 15111. Farnesyl transferase inhibitors, such as FTI-277 and R1 15777, interfere with the translocation of HRAS to the cell membrane (Lerner et al, Oncogene 15: 11, 1997; Ochiai et al, Blood 102:9, 2003; Zhu et al, Blood 105: 12, 2005). S-trans, trans-farnesylthiosalicylic acid, a new, synthetic, farnesylated, rigid carboxylic acid derivative has been shown to dislodge HRAS from its membrane anchorage domains and accelerate HRAS degradation (Gana-Weisz et al, Clinical Cancer Research 8, 2002). A naturally occurring organosulfur compound in garlic, DADS, has been shown to be effective in the treatment of experimental brain C6 glioma in a rat model (Perkins et al, Mol Brain Res. Ill, 2003). Additionally, dominant inhibitory mutants have been created for each of the three RAS isoforms that substitute serine 17 with aspargine (Matallanas et al, J. Biol. Chem. 278:1, 2003).

At least one known inhibitor of KRAS can be suitably employed as an adjunctive therapy. S-trans, trans-farnesylthiosalicylic acid, a new, synthetic, farnesylated, rigid carboxylic acid derivative has been shown to dislodge KRAS from its membrane anchorage domains and accelerate KRAS degradation (Ji et al, J. Biol. Chem. 282: 19, 2007). To practice the coordinate administration methods of this disclosure, an oligonucleotide as described herein is administered, simultaneously or sequentially, in a coordinated treatment protocol with one or more of the secondary or adjunctive therapeutic agents disclosed herein. The coordinate administration can be done in any order, and there can be a time period while only one or both active therapeutic agents, individually or collectively, exert their biological activities. A distinguishing aspect of such coordinated treatment methods is that the oligonucleotide present in a composition elicits some favorable clinical response, which can be in conjunction with a secondary clinical response provided by the secondary therapeutic agent. For example, the coordinated administration of the oligonucleotide with a secondary therapeutic agent as disclosed herein can yield an enhanced (synergistic) therapeutic response beyond the therapeutic response elicited by the purified dsRNA or secondary therapeutic agent alone.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition must be sterile and can be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams known in the art.

The oligonucleotide compounds of the invention can also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Non-limiting examples of United States patents that teach the preparation of such uptake, distribution and/or absorption-assisting formulations include U.S. Pat. Nos. 5,108,921; 5,354,844; 5,416,016; 5,459,127; 5,521,291; 5,543,165; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

For treating tissues in the central nervous system, administration can be made by, e.g., injection or infusion into the cerebrospinal fluid. Administration of antisense RNA into cerebrospinal fluid is described, e.g., in U.S. Pat. No. 7,622,455, which is incorporated by reference in its entirety. When it is intended that an oligonucleotide compound (e.g., an ASO or SSO) will be administered to cells of the central nervous system, administration can be with one or more agents that can promote penetration of the oligonucleotide compound across the blood-brain barrier. Injection can be made, e.g., in the entorhinal cortex or hippocampus. See also U.S. Pat. Nos. 6,632,427 and 6,756,523 for additional disclosures relating to direct delivery to the brain, each patent which is incorporated by reference in its entirety. For treating cardiac tissues, administration can be made by, e.g., injection or infusion into the bloodstream. The injection can be administered by the following routes: intraperitoneal injection, subcutaneous injection, intradermal injection, intravenous injection, intramuscular injection, intra-arterial injection, or a combination thereof. In one embodiment, administration into the bloodstream is useful.

Formulations useful for topical administration include those in which the oligonucleotide compounds of the invention are in admixture with a topical delivery agent such as lipids, liposomes, fatty acids, fatty acid esters, steroids, chelating agents and surfactants. Exemplary lipids and liposomes include neutral (e.g. diolcoyl-phosphatidyl ethanolamine (DOPE), dimyristoylphosphatidyl choline (DMPC), distearolyphosphatidyl choline) negative (e.g. dimyristoylphosphatidyl glycerol (DMPG)) and cationic (e.g. diolcoyltetramethyl-aminopropyl (DOTAP), and diolcoyl-phosphatidyl ethanolamine (DOTMA)). For topical or other administration, oligonucleotide compounds of the invention can be encapsulated within liposomes or can form complexes thereto, such as cationic liposomes. Alternatively, oligonucleotide compounds can be complexed to lipids, such as cationic lipids. Exemplary fatty acids and esters, pharmaceutically acceptable salts thereof, and their uses are further described in U.S. Pat. No. 6,287,860, which is hereby incorporated by reference in its entirety.

The formulation of therapeutic compositions and their subsequent administration (dosing) is within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages can vary depending on the relative potency of individual oligonucleotides, and can be estimated based on EC50s found to be effective in in vitro and in vivo animal models. In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, or at least about 500 mg/kg body weight.

In one embodiment, the oligonucleotide compound can be administered to the subject one time (e.g., as a single injection or deposition). Alternatively, administration can be once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. For example, the dosage can be given once or more daily, weekly, monthly or yearly, or even once every 2 to 20 years. In one embodiment, two or more combined oligonucleotide compounds, therapeutics, and the like can be used together in combination or sequentially. The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect required for uses described herein; and rate of excretion. Persons of ordinary skill in the art can easily estimate repetition rates for dosing based on measured residence times and concentrations of the drug in bodily fluids or tissues. Following successful treatment, it can be desirable to have the patient undergo maintenance therapy to prevent the recurrence of the disease state, wherein the oligonucleotide compound is administered in maintenance doses, ranging from at least about 0.1 mg/kg body weight to about 10 mg/kg of body weight, once or more daily, to once every 2-20 years. Certain injected dosages of antisense oligonucleotides, for example, are described, in U.S. Pat. No. 7,563,884, which is hereby incorporated by reference in its entirety.

Oligonucleotides of the invention can be manufactured for delivery using a recombinant viral vector. A “recombinant viral vector” can refer to a recombinant polynucleotide vector comprising one or more heterologous sequences (i.e., nucleic acid sequence not of viral origin).

A “recombinant AAV vector (rAAV vector)” can refer to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e, AAV Rep and Cap proteins). When a rAAV vector is incorporated into a larger polynucleotide (e.g., in a chromosome or in another vector such as a plasmid used for cloning or transfection), then the rAAV vector can be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

An “rAAV virus” or “rAAV viral particle” can refer to a viral particle composed of at least one AAV capsid protein and an encapsidated rAAV vector genome.

A “recombinant adenoviral vector” can refer to a polynucleotide vector comprising one or more heterologous sequences (i.e., nucleic acid sequence not of adenovirus origin) that are flanked by at least one adenovirus inverted terminal repeat sequence (ITR). In some embodiments, the recombinant nucleic acid is flanked by two inverted terminal repeat sequences (ITRs). Such recombinant viral vectors can be replicated and packaged into infectious viral particles when present in a host cell that is expressing essential adenovirus genes deleted from the recombinant viral genome (e.g., E1 genes, E2 genes. E4 genes, etc.). When a recombinant viral vector is incorporated into a larger polynucleotide (e.g., in a chromosome or in another vector such as a plasmid used for cloning or transfection), then the recombinant viral vector can be referred to as a “pro-vector” which can be “rescued” by replication and encapsidation in the presence of adenovirus packaging functions. A recombinant viral vector can be in any of a number of forms, including, but not limited to, plasmids, linear artificial chromosomes, complexed with lipids, encapsulated within liposomes, and encapsidated in a viral particle, for example, an adenovirus particle. A recombinant viral vector can be packaged into an adenovirus virus capsid to generate a “recombinant adenoviral particle.”

A “recombinant lentivirus vector” can refer to a polynucleotide vector comprising one or more heterologous sequences (i.e., nucleic acid sequence not of lentivirus origin) that are flanked by at least one lentivirus terminal repeat sequences (LTRs). In some embodiments, the recombinant nucleic acid is flanked by two lentiviral terminal repeat sequences (LTRs). Such recombinant viral vectors can be replicated and packaged into infectious viral particles when present in a host cell that has been infected with a suitable helper function. A recombinant lentiviral vector can be packaged into a lentivirus capsid to generate a “recombinant lentiviral particle.”

A “recombinant herpes simplex vector (recombinant HSV vector)” can refer to a polynucleotide vector comprising one or more heterologous sequences (i.e., nucleic acid sequence not of HSV origin) that are flanked by HSV terminal repeat sequences. Such recombinant viral vectors can be replicated and packaged into infectious viral particles when present in a host cell that has been infected with a suitable helper function. When a recombinant viral vector is incorporated into a larger polynucleotide (e.g., in a chromosome or in another vector such as a plasmid used for cloning or transfection), then the recombinant viral vector can be referred to as a “pro-vector” which can be “rescued” by replication and encapsidation in the presence of HSV packaging functions. A recombinant viral vector can be in any of a number of forms, including, but not limited to, plasmids, linear artificial chromosomes, complexed with lipids, encapsulated within liposomes, and encapsidated in a viral particle, for example, an HSV particle. A recombinant viral vector can be packaged into an HSV capsid to generate a “recombinant herpes simplex viral particle.”

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” can refer to a polynucleotide that is introduced into a cell and can be transcribed into RNA and optionally, translated and/or expressed under appropriate conditions. In aspects, it confers a property to a cell into which it was introduced, or otherwise leads to a therapeutic or diagnostic outcome. In another aspect, it can be transcribed into a molecule that modulates splicing, such as an ASO or oligonucleotide as described herein.

Embodiments can also comprise compositions and methods to directly correct KRAS G60G silent mutations, such as using a CRISPR complex, such as CRISPR-Cas9, with KRAS mutant-specific sgRNA, in order to convert the original KRAS GQ60GK into the non-functional Q61K.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a Cas gene and specifically designed CRISPRs, the organism's genome can be cut and modified at virtually any location. A number of methods exist for expressing the guide strand or Cas protein, including inducible expression of one or both. A number of methods exist for introducing the guide strand and Cas protein into cells including viral transduction, injection or micro-injection, nano-particle or other delivery, uptake of proteins, uptake of RNA or DNA, uptake of combination of protein and RNA or DNA. Combinations of methods can also be used, simultaneously or in sequence. Multiple rounds of delivery of RNA, DNA or protein can occur with or without further protein expression. Methods of preparing compositions for use in genome editing using the CRISPR/Cas systems are described in detail in WO 2013/176772 and WO 2014/018423, which are specifically incorporated by reference herein in their entireties.

The term “CRISPR activity” can refer to an activity associated with a CRISPR system. Examples of such activities are double-stranded nuclease, nickase, transcriptional activation, transcriptional repression, nucleic acid methylation, nucleic acid demethylation, and recombinase.

The term “CRISPR system” can refer to a collection of CRISPR proteins and nucleic acid that, when combined, result in at least CRISPR associated activity (e.g., the target locus specific, double-stranded cleavage of double-stranded DNA).

The term “CRISPR complex” can refer to the CRISPR proteins and nucleic acid (e.g., RNA) that associate with each other to form an aggregate that has functional activity. An example of a CRISPR complex is a wild-type Cas9 (sometimes referred to as Csn1) protein that is bound to a guide RNA specific for a target locus.

The term “CRISPR protein” can refer to a protein comprising a nucleic acid (e.g., RNA) binding domain nucleic acid and an effector domain (e.g., Cas9, such as Streptococcus pyogenes Cas9). The nucleic acid binding domains interact with a first nucleic acid molecules having a region that can hybridize to a target nucleic acid (e.g., a guide RNA) or allows for the association with a second nucleic acid having a region that can hybridize to the target nucleic acid (e.g., a crRNA). CRISPR proteins can also comprise nuclease domains (i.e., DNase or RNase domains), additional DNA binding domains, helicase domains, protein-protein interaction domains, dimerization domains, as well as other domains.

CRISPR protein also can refer to proteins that form a complex that binds the first nucleic acid molecule referred to herein. Thus, one CRISPR protein can bind to, for example, a guide RNA and another protein can have endonuclease activity. These are considered to be CRISPR proteins because they function as part of a complex that performs the same functions as a single protein such as Cas9.

In embodiments, CRISPR proteins can contain nuclear localization signals (NLS) that allow them to be transported to the nucleus.

### Kits

The invention also provides kits for treatment of a subject with a RAS-associate disease or condition. In embodiments, the kit comprises at least an oligonucleotide compound, packaged in a suitable container, together with instructions for its use. In one embodiment, the invention provides for a kit for the treatment of a RAS-associated disease or condition, the kit comprising an oligonucleotide compound discussed herein. In one embodiment, the invention provides for a kit for the treatment of a RAS-associated disease or condition, the kit comprising at least two oligonucleotide compounds discussed herein. In one embodiment, the RAS-associated disease or condition is cancer. In one embodiment, the oligonucleotide compound comprises an oligonucleotide of a sequence described herein. In one embodiment, the oligonucleotide compound comprises at least one modification described herein. In some embodiments, the kits will contain at least one oligonucleotide compound (e.g., an ASO or SSO), such as shown herein, or a cocktail of antisense molecules comprising a combination of oligonucleotides described herein.

While the embodiments of the invention are described with reference to various implementations and exploitations, it will be understood that these embodiments are illustrative and that the scope of the inventions is not limited to them. Many variations, modifications, additions, and improvements are possible. Further still, any steps described herein can be carried out in any order, and any steps can be added or deleted. Support for the invention and additional embodiments of the invention can be found in the attached documents which are expressly incorporated herein in their entirety by reference hereto. Also, the phraseology and terminology used herein is for the purpose of description and cannot be regarded as limiting. The use of “including,” “comprising,” or “having,” “containing,” “involving,” and variations thereof herein, is meant to encompass the items listed thereafter and equivalents thereof as well as additional items.

Unless otherwise defined, technical and scientific terms used herein have the same meaning as understood by one of ordinary skill in the art to which this invention belongs. Exemplary methods and materials are described herein, although methods and materials similar or equivalent to those described herein can also be used in the practice or testing of the invention.

As will be apparent to one of ordinary skill in the art from a reading of this disclosure, the embodiments of the disclosure can be embodied in forms other than those specifically disclosed herein. The embodiments described herein are, therefore, to be considered as illustrative and not restrictive. Those skilled in the art will recognize, or be able to ascertain, using no more than routine experimentation, numerous equivalents to the specific embodiments described herein. The scope of the invention is as set forth in the appended claims and equivalents thereof, rather than being limited to the examples contained in the foregoing description.

Publications and other references mentioned herein are incorporated by reference in their entirety, as if each individual publication or reference were specifically and individually indicated to be incorporated by reference. Publications and references cited herein are not admitted to being prior art.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

KRAS is a mutated oncogene in cancer. Although KRAS G12C-specific Inhibitors show responses in patients with lung cancer, other variants, for example Q61K mutations are not targetable.

Considering that KRAS mutations are also found as acquired resistant mechanisms to EGFR-tyrosine kinase inhibitors, we created intrinsic KRAS mutations in EGFR mutant lung cancer cell line PC9 by CRISPR genome editing. Through these models, we unexpectedly discovered the significance of silent mutations and splicing vulnerabilities in Q61 mutant RAS cancers. Models with KRAS Q61K underwent alternative splicing due to the similarity to conserved motif of splicing donor site which resulted in causing early stop codon (and as such a non-functional protein), whereas KRAS Q61K+silent mutation (G60G+Q61K, also referred to as GQ60GK) prevented from alternative splicing. In 3 independent pan-cancer cohorts, we confirmed that KRAS Q61K always co-exists with KRAS G60G silent mutation. Additionally, we found that sequences around RAS Q61 are hotspots of the motifs for exonic splicing enhancers (ESE), and genome editing around these areas can induce whole exon 3 skipping and subsequent early stop codon by interrupting these motifs.

We designed mutant-specific antisense oligos targeting the hotspot of ESE in pre-mRNA of KRAS, NRAS, or HRAS Q61 cancer. Skipping whole exon 3 was induced in RAS Q61 mutant pan-cancer cell lines resulting in pre-mature termination. In vitro data demonstrated growth inhibition and decreased downstream signals including pERK and pAKT.

As described herein, mutant-specific morpholino antisense oligos can be used as a new therapy targeting RAS Q61 mutant pan-cancers.

Currently, only KRAS G12C-specific inhibitors, which lock the KRAS protein in its inactive GDP-bound state, showed promising data in clinical trials, but there are no direct inhibitors for KRAS Q61, NRAS, or HRAS cancer. Our approach targeting splicing vulnerability is a new concept, and is applicable to any cancer with a mutation in codon 61 of KRAS, NRAS, or HRAS. Of note, the use of mutant-specific morpholino antisense oligos inhibits only mutant pre-mRNA and does not affect wild type pre-RNA intact. As such, these strategies are Q61 RAS mutant specific and do not impact wild type KRAS. Thus, this approach can have a wider therapeutic index than MEK inhibitors. MEK inhibitors are used in KRAS mutant cancers, but have no mutant selectively and are toxic (due to inhibiting MEK in normal tissues). Considering that treatments with antisense oligo nucleotides for spinal muscular atrophy and morpholino antisense oligos for Duchenne muscular dystrophy have already been approved by FDA, our strategy can also be therapeutically viable.

Described herein are mutant-specific morpholino antisense oligos and their use for the treatment of RAS (KRAS, HRAS or NRAS) Q61 mutant cancers.

### Example 2—KRAS Silent Mutations Uncover a Treatment Approach for RAS 061 Cancers

### Abstract

Targeted therapies in cancers with non-synonymous somatic mutations, focal amplifications, and translocations can improve survival¹. RAS family members, including KRAS, NRAS, and HRAS, are the most frequently mutated oncogenes in human cancers. Although KRAS G12C-specific inhibitors show clinical activity in patients with lung cancer²⁻⁴, there are no direct inhibitors of NRAS, HRAS or non-G12C KRAS variants. Here we uncover a silent mutation in KRAS G60G for a functional KRAS Q61K. In the absence of a silent mutation at G60 in KRAS Q61K, a cryptic splice donor site is formed, leading to alternative splicing and a truncated non-functional protein. The presence of a G60G silent mutation eliminates the splice donor site, yielding a functional KRAS Q61K variant. A striking concordance of KRAS Q61K and a G60G/A59A silent mutation was detected in three independent pan-cancer cohorts. We further reveal that the region around RAS Q61 is enriched in exonic splicing enhancer (ESE) motifs and design mutant-specific oligos to interfere with ESE-mediated splicing, rendering the RAS Q61 protein non-functional in a mutant selective manner. The induction of aberrant splicing by mutant-selective antisense oligos demonstrated therapeutic effects in vitro and in vivo. By studying splicing necessary for a functional KRAS Q61K, we uncover a mutant selective RAS Q61 cancer treatment strategy, as well as expose a mutant-specific vulnerability, which, without wishing to be bound by theory, can be therapeutically exploited in other genetic contexts.

### Introduction

The effects of non-synonymous mutations, which alter the amino acid sequence of a protein, have been investigated for their potential to disrupt normal human biology and/or cause cancer. The vulnerabilities of oncogenic non-synonymous somatic mutations or translocations have since been targeted with specific drugs^{1,5,6}. In contrast, the clinical significance of synonymous (silent) mutations remains elusive, despite some evidence that silent mutations are involved in splicing, RNA stability, RNA folding, translation or co-translational protein folding^{7,8}. The role of synonymous mutations in cancer etiology has not been systematically studied, and as such silent mutations are for the most part disregarded as noise in clinical mutational analyses.

Mutations in the RAS family genes are found in up to 20% of cancers: KRAS in non-small cell lung (NSCLC), colorectal, and pancreatic cancers; NRAS in melanoma, colon cancer, and leukemia; and HRAS in bladder, breast, and thyroid cancers.⁹ RAS proteins are guanosine triphosphatases (GTPases) acting as binary switches that cycle between inactive (GDP bound) and active (GTP bound) states. Activated RAS proteins stimulate the downstream Mitogen-activated Protein Kinase (MAPK) pathway including MEK and ERK. Somatic mutations in RAS increase GTP-bound RAS, aberrantly activating MAPK signaling.

The development of targeted therapies for RAS mutant cancers has been complex. MEK inhibitors combined with chemotherapy in KRAS mutant NSCLC have limited efficacy¹⁰. Combined use of a MEK inhibitor with receptor tyrosine kinase (RTK) inhibitors, given RTK-mediated feedback activation of the MAPK/ERK pathway, has also been proposed but toxicity and lower efficacy of the MEK inhibitor—due to lack of mutant selectivity—is a challenge¹¹⁻¹³. Allosteric inhibitors targeting SHP2, a non-receptor protein tyrosine phosphatase that transduces signaling from RTKs to promote the activation of RAS, are in clinical development¹⁴. The first successful RAS-targeted therapies involve use of KRAS G12C-specific covalent inhibitors that lock the protein in its inactive, GDP-bound state². To date, phase-I clinical trials have demonstrated encouraging clinical activity of these compounds in patients with NSCLC^{3,4,15}. Another approach is to target SOS1, a guanine exchange factor for KRAS that binds and activates GDP-bound RAS-proteins at its catalytic binding site and in this way promotes exchange of GDP for GTP. However, KRAS Q61 mutants, which lack intrinsic GTP hydrolysis activity^{16,17}, aren't responsive to SOS1 inhibitors, warranting the development of alternative Q61X-selective therapeutic strategies.

### Results

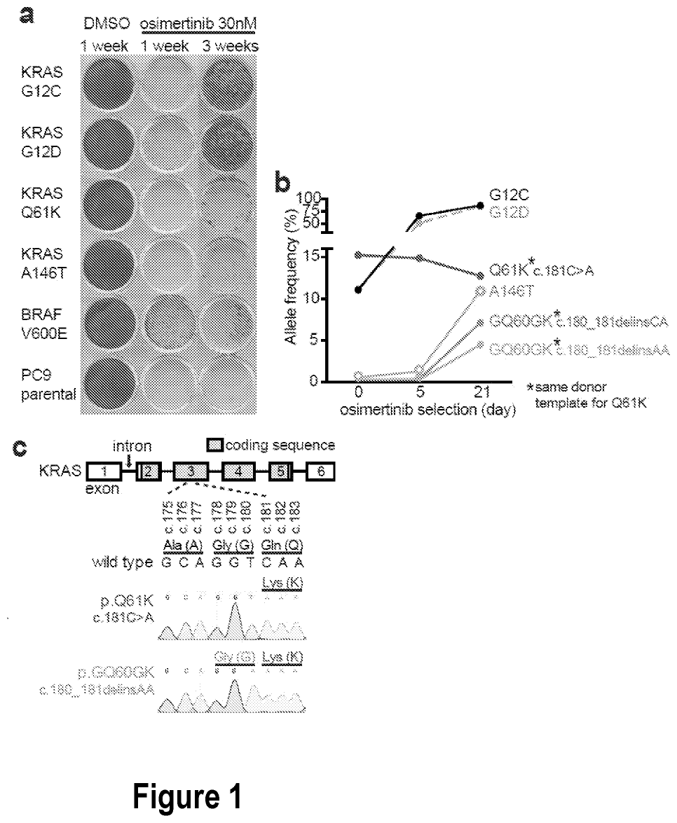

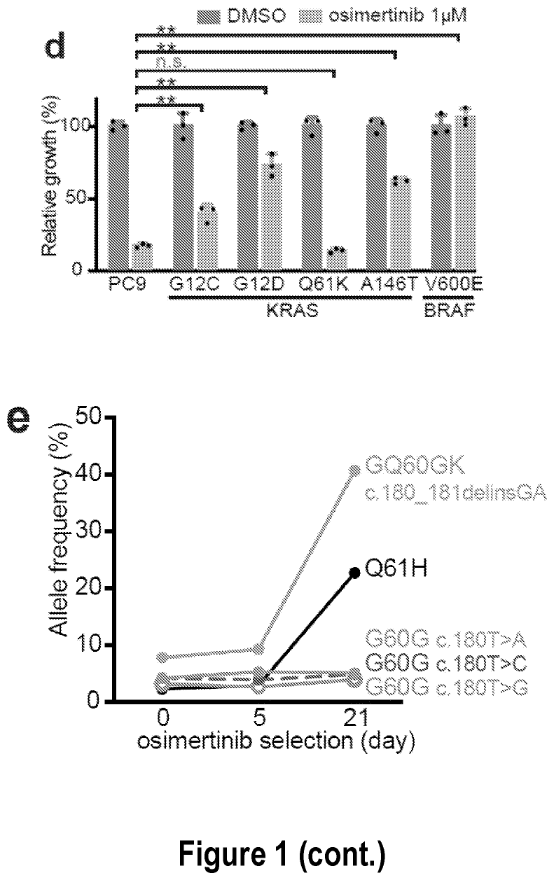

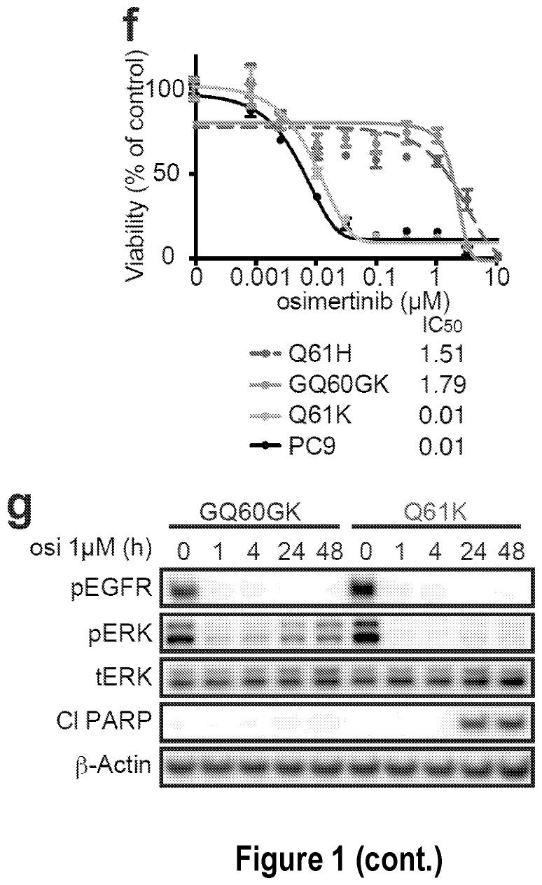

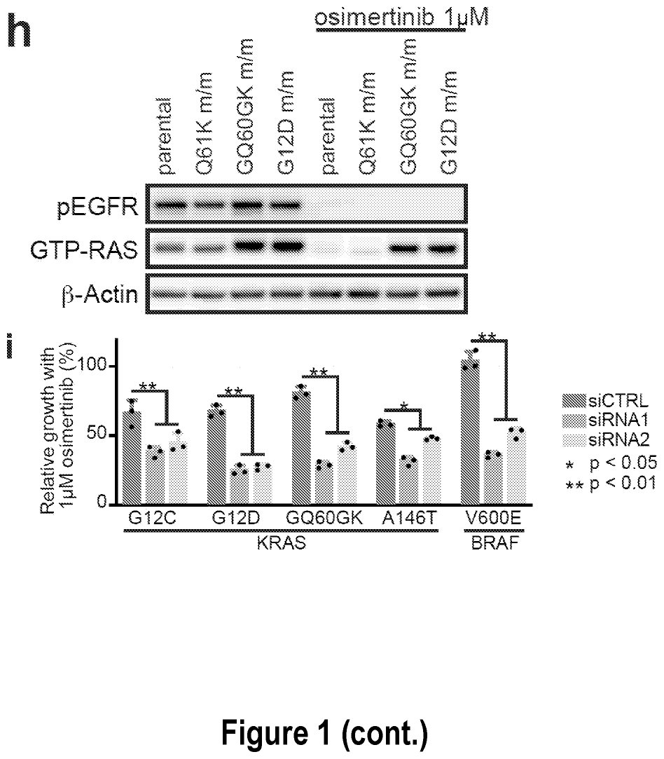

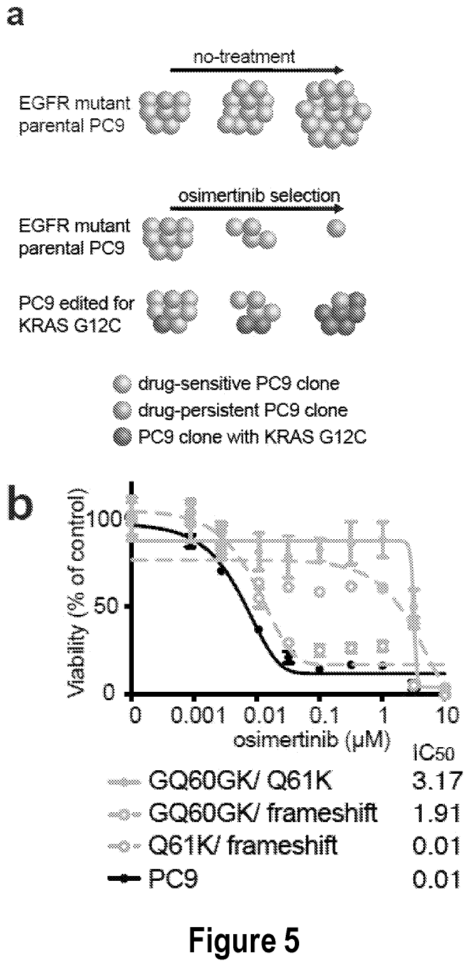

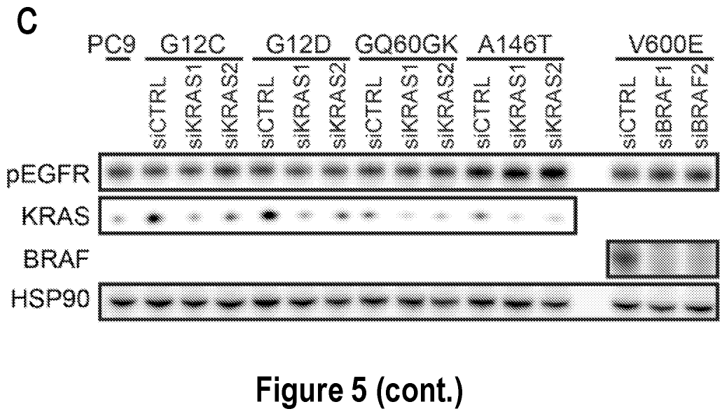

Acquired somatic mutations in KRAS G12C, G12D, Q61K, A146T, and BRAF V600E drive resistance to epidermal growth factor receptor (EGFR)-tyrosine kinase inhibitor osimertinib in EGFR mutant lung cancers^{18,19}. To model these events in vitro, we introduced mutations into KRAS or BRAF in the EGFR mutant lung cancer cell line, PC9, using CRISPR-Cas9 homology directed repair (HDR), and selected for resistance imparting clones. As expected, osimertinib treatment led to a selective increase in cells harboring different KRAS mutations or BRAF V600E but not in the parental cells (FIG. 1 panel a, and FIG. 5 panel a). We monitored mutant allele frequencies (AFs) over the course of drug selection, using targeted next generation sequencing (NGS), and noted that while the AFs of KRAS G12C, G12D, and A146T increased over time under osimertinib selection pressure, the AF of KRAS Q61K instead decreased (FIG. 1 panel b). Unexpectedly, we noted that the AFs of two other KRAS Q61K alleles, both containing a concurrent silent mutation at G60, GQ60GK (c.180_181 delinsCA or AA), increased sharply in response to drug treatment (FIG. 1 panels b, c). The GQ60GK double mutants emerged from a CRISPR-Cas9 editing event that used the same donor template designed for Q61K (c.181C>A) single mutant and can be the result of the error-prone non-homologous end joining repair. When tested for osimertinib sensitivity, single-cell clones of PC9 cells harboring KRAS Q61K without this silent mutation failed to impart resistance, exhibiting growth inhibition comparable to that of parental PC9 cells. This was in stark contrast to the KRAS G12C, G12D, A146T, or BRAF V600E clones that were largely unaffected by 1 μM osimertinib treatment (FIG. 1 panel d). Considering the 3 possible G60 mutant variants (c.180T>A, C, or G), we edited PC9 cells using donor templates for KRAS GQ60GK (c. 180_181 delinsGA), KRAS G60G alone (c.180T>A, C, or G) and another non-synonymous mutation at codon 61, KRAS Q61H. Under osimertinib pressure, the AFs of GQ60GK (c.180_181 delinsGA) and Q61H increased, but AFs of G60G silent mutations alone remained unchanged (FIG. 1 panel e). Heterozygous KRAS GQ60GK or Q61H mutations alone were sufficient to cause resistance to osimertinib in the PC9 models (FIG. 1 panel f and FIG. 5 panel b).

To examine the gain of function effect of the KRAS GQ60GK mutation, we evaluated its protein product and impact on downstream signaling. Following osimertinib treatment, persistent ERK1/2 activation was present in PC9 cells expressing KRAS GQ60GK c. 180_181 delinsAA but not in cells expressing KRAS Q61K alone (FIG. 1 panel g), confirming the uncoupling of EGFR inhibition from that of ERK 1/2 in the double-mutant only. In addition, KRAS GQ60GK exhibited RAS-GTP levels similar to those of KRAS G12D at baseline, which were minimally affected by osimertinib treatment (FIG. 1 panel h). In contrast, osimertinib treatment led to a decrease in RAS-GTP in the KRAS Q61K single mutant or in KRAS wild type parental PC9 cells. Reciprocally, knocking down KRAS or BRAF genes by siRNA resensitized resistant PC9 models to osimertinib, corroborating causality between KRAS or BRAF mutations and osimertinib resistance (FIG. 1 panel i and FIG. 5 panel c). Collectively our findings indicate a requirement of a silent mutation in KRAS G60G for the biological function of KRAS Q61K.

### KRAS GQ60GK is Present in Three Pan-Cancer Cohorts

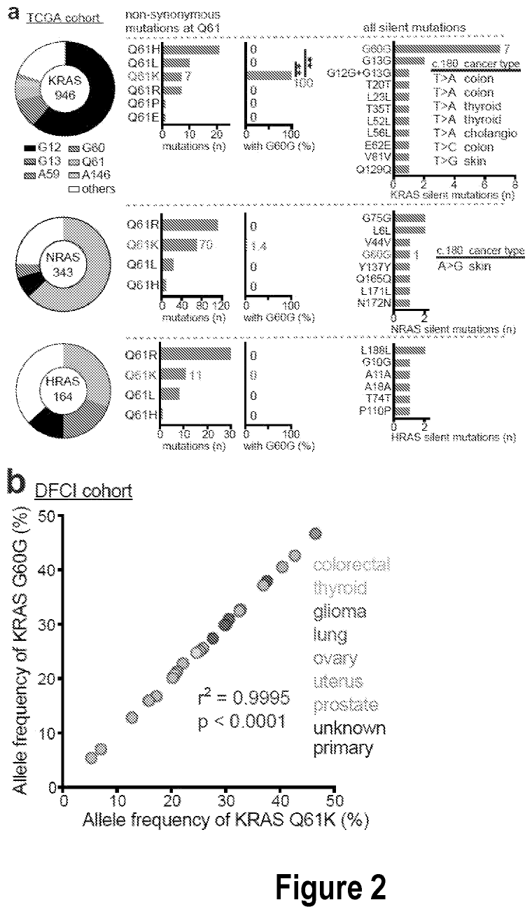

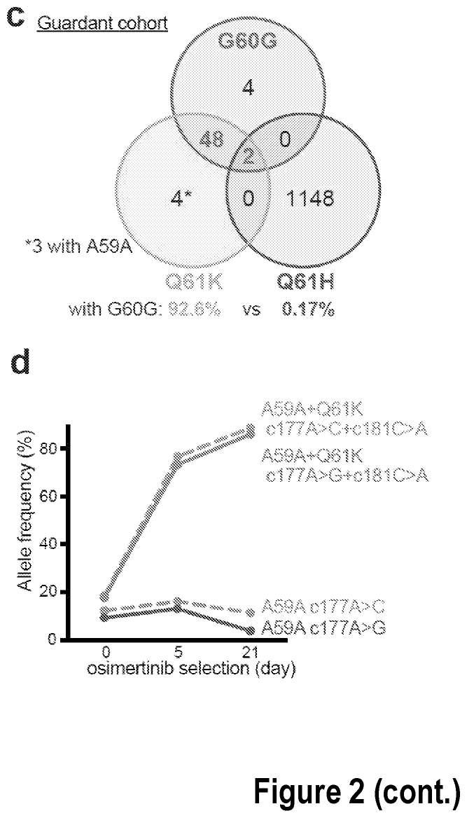

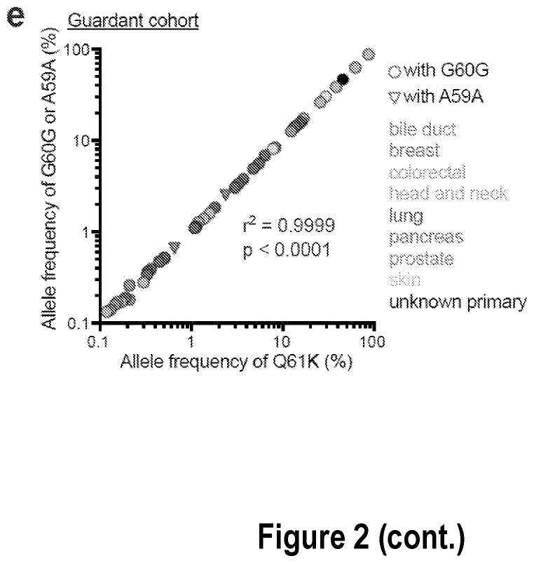

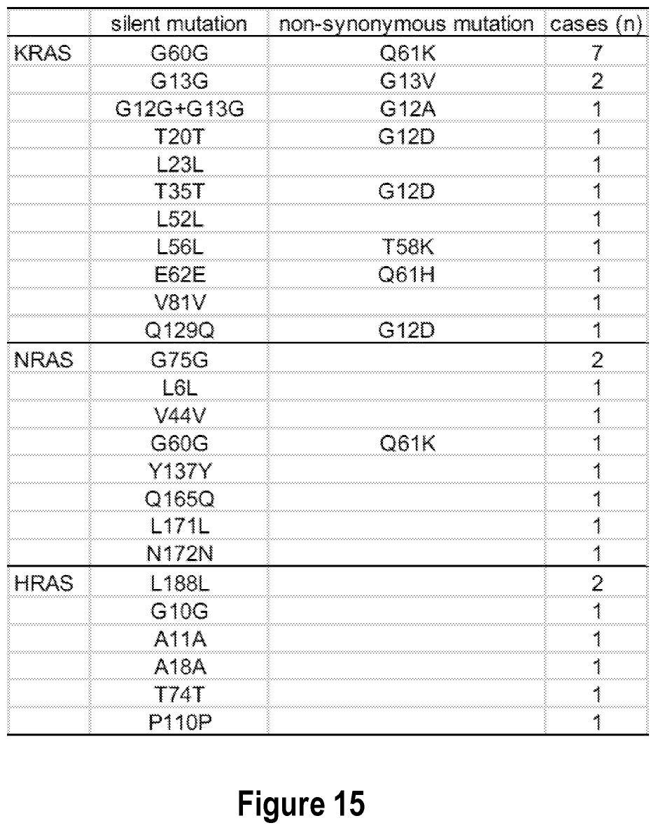

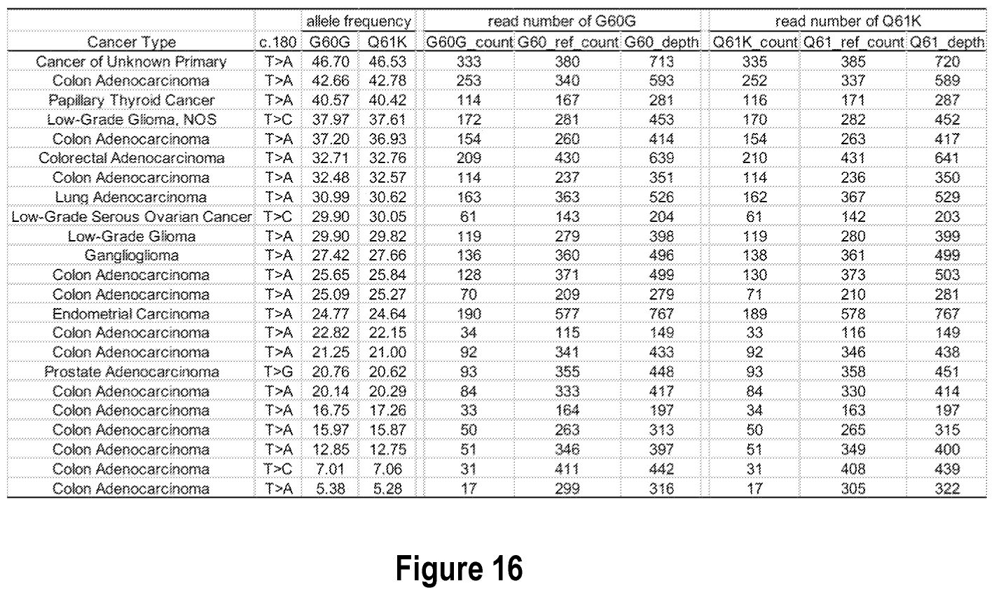

We surveyed The Cancer Genome Atlas (TCGA) data on KRAS and other RAS family member mutants for the frequency of silent co-mutations. Q61 was the most frequently mutated codon in NRAS and HRAS, and the third most common mutation in KRAS (FIG. 2 panel a, and FIG. 15). Silent mutations were found in 2-4% of all RAS family mutant cancers: 18 KRAS cases, 10 NRAS cases, and 7 HRAS cases. Notably, however, all 7 cancers with KRAS Q61K also contained KRAS G60G (c.180T>A, C, or G) silent mutations. This co-occurrence was unique to KRAS Q61K among KRAS mutations and G60G was only found once among 81 NRAS or HRAS Q61K mutant cancers (FIG. 2 panel a). To expand upon this finding, we examined a pan-cancer cohort (n=25,252) sequenced by targeted NGS at the Dana-Farber Cancer Institute. Among these, we identified 23 cases of KRAS Q61K and all of them contained a silent mutation at G60G (FIG. 2 panel b), including the case of a patient whose lung adenocarcinoma acquired KRAS GQ60GK mutation after osimertinib treatment¹⁸ used to model in vitro resistance (FIG. 1). There was a high degree of concordance between the allelic frequency of the mutations leading to KRAS Q61K and KRAS G60G (FIG. 2 panel b and FIG. 16).

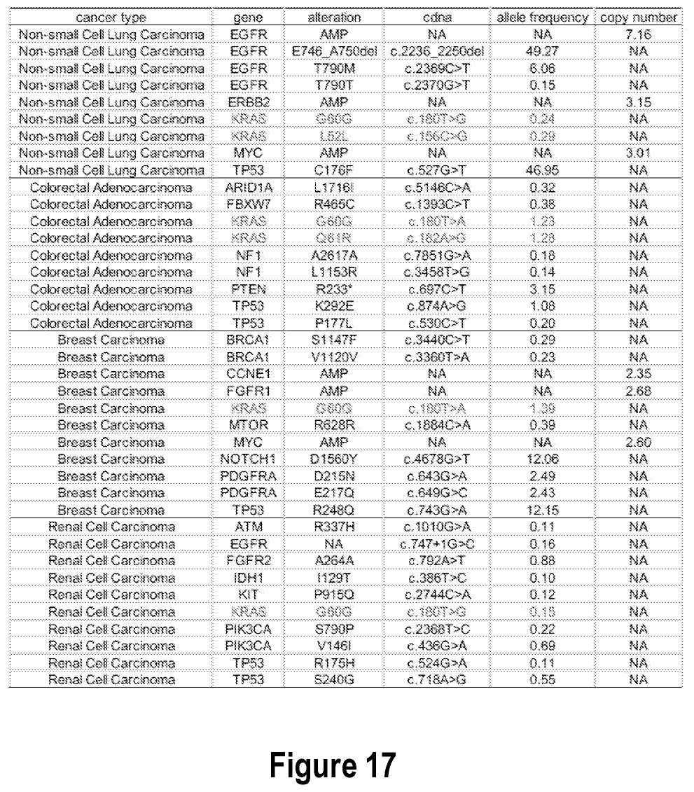

Given the widespread use of liquid biopsies, using plasma to detect genomic drivers and mechanisms of resistance in circulating tumor DNA and for monitoring the effect of treatment^{20,21}, we studied KRAS Q61K, G60G, and Q61H cancers in the Guardant Health clinical cohort, analyzed by targeted NGS (Guardant360). The co-occurrence of the KRAS G60G silent mutations was significantly higher in KRAS Q61K than in KRAS Q61H cancers (FIG. 2 panel c). Of the 54 cancers with KRAS Q61K, 50 (93%) cases had KRAS G60G silent mutations, compared to only 2/1148 (0.17%) of KRAS Q61H cancers (p<0.0001). Of note, both KRAS Q61H cancers harboring a KRAS G60G silent mutation also contained a concomitant KRAS Q61K mutation (FIG. 2 panel C). In this cohort, three of the four KRAS Q61K cancers that did not have a KRAS G60G mutation contained a different silent mutation, KRAS A59A (c.177A>A or G). Interrogating the function of the mutation using the osimertinib selection assay of CRISPR-modified PC9, we noted that the AF of A59A alone did not increase under drug selection (FIG. 2 panel d). However, in cells with a silent mutation in A59A in cis with Q61K, the allelic fraction increase mirrored that seen with GQ60GK (FIG. 1 panel e and 2 panel d). Data on AFs of KRAS G60G and A59A were obtained in 45 cases with KRAS Q61K; in the 45 cases, the AFs of KRAS Q61K correlated with KRAS G60G or A59A silent mutations (FIG. 2 panel e). In the remaining 9 cases without available AFs, the silent mutation in G60G and A59A was confirmed to be in cis with Q61K by manual review for analytic accuracy. Four cases had only KRAS G60G silent mutations without KRAS Q61K: NSCLC with EGFR del746_750+T790M, breast cancer with BRCA1 S1147F, colon cancer with KRAS Q61R, and renal cell carcinoma with low tumor shed (AFs <1%) and without an apparent driver mutation (FIG. 17).

### KRAS G60G is Required for Proper Splicing in KRAS Q61K

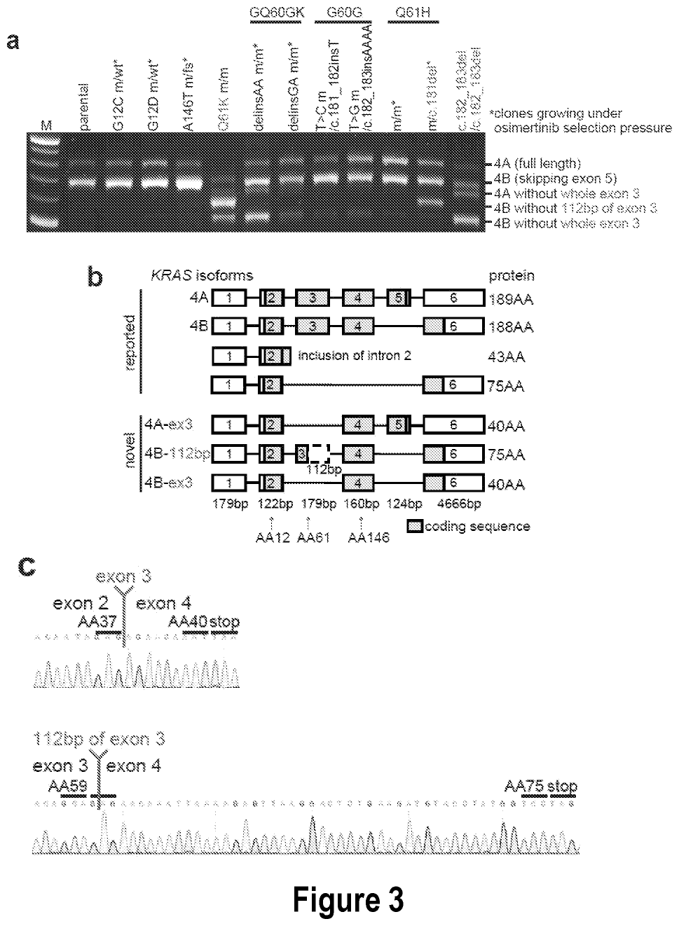

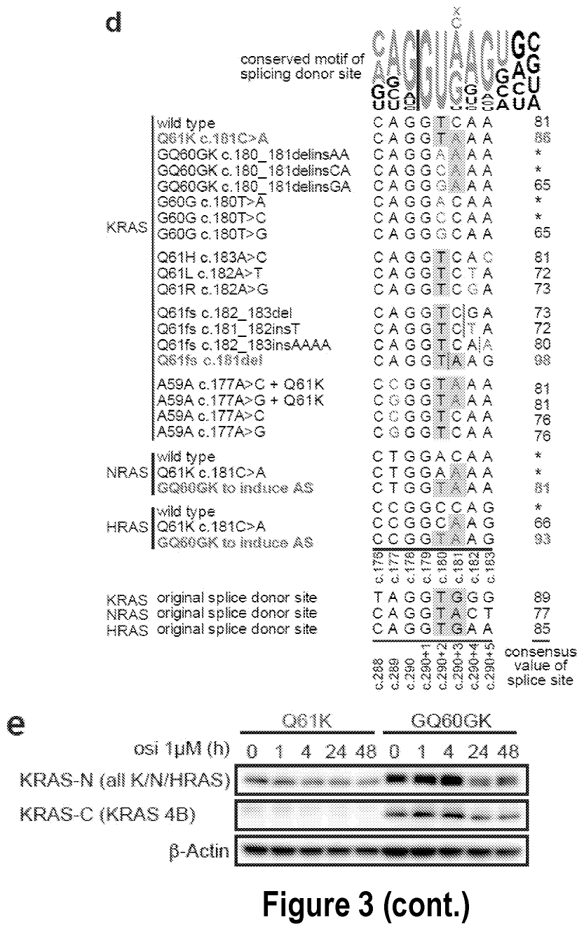

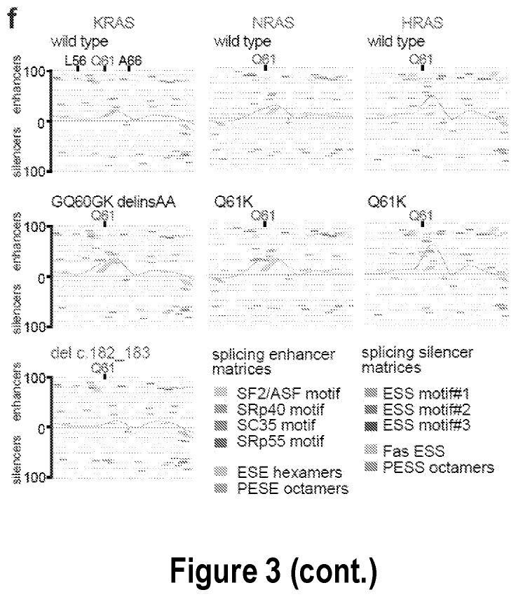

To validate the mechanism defining the reliance of functional KRAS Q61K on silent mutations in KRAS G60G, we amplified the cDNA of KRAS from CRISPR-modified PC9 clones emerging in our screen, following osimertinib or control treatment (FIG. 3 panel a). In addition to the two previously documented isoforms 4A (full length KRAS) and 4B (lacking exon 5)²² observed in parental PC9 cells and clones expressing KRAS G12C, G12D, A146T, G60G, and Q61H mutations, two transcript isoforms were identified in clones harboring KRAS GQ60GK or Q61K. These isoforms are characterized by the absence of 112 bp within exon 3, or skipping of the entire exon 3 (FIG. 3 panels a-c). Without wishing to be bound by theory, neither isoform will be translated to a functional KRAS Q61K, due to a frameshift introducing an early stop codon.

The sequence of wild-type KRAS around Q61 shows a high consensus with the conserved motif of a splice donor site²³, deviating only at c. 181 (FIG. 3 panel d). The mutation resulting in KRAS Q61K (c.181C>A) simultaneously introduces a putative cryptic splice donor site at that location, with a consensus value (86) that is equivalent to the canonical splice donor site between exon 3 and intron 3 (89), and as such could result in an aberrant splicing event producing either no protein or a non-functional Q61K variant observed in our screen. To validate this, we analyzed the protein products of KRAS GQ60GK and KRAS Q61K by western blotting following an osimertinib challenge, using antibodies that bind either the N-terminus (common to KRAS, NRAS and HRAS) or the C-terminus (unique to KRAS 4B). While the N-terminal antibody detected RAS in both the KRAS Q61K and GQ60GK cells, the C-terminal antibody detected none/minimal RAS in the KRAS Q61K cells and more robust expression in the KRAS GQ60GK cells (FIG. 3 panel e). Collectively, these data indicate that KRAS Q61K alone cannot produce full-length KRAS protein competent to impart osimertinib resistance (FIGS. 3 panel e and 1 panel h).

Another one of our CRISPR edited PC9 clones harbors a heterozygous deletion of c.181 along with Q61H (FIG. 3 panel a). This single base pair deletion similarly introduces a cryptic splice site with a high consensus value (98), leading us to conclude that aberrant splicing at this site is responsible for deleting 112 bp of exon 3 in this isoform (FIG. 3 panels a, d), creating a frameshift. Silent mutations at KRAS G60G (c.180T>A, C, or G) disrupt the cryptic splice donor site introduced by the KRAS Q61K mutation, as evidenced by its low consensus value relative to the conserved splice site (FIG. 3 panel d). Other KRAS Q61X variants such as Q61H/L/R do not generate a cryptic splice donor site because these mutations occur in c.182 or 183 (FIG. 3 panel d). KRAS A59A (c.177A>A or G) silent mutations (FIG. 2 panel c), like KRAS G60G silent mutations, decrease the splice site consensus values in KRAS Q61K and as such produce a functional KRAS Q61K (FIGS. 2 panel d and 3 panel d). In NRAS or HRAS, c. 180 in the wild-type sequence is an A or C, and consequently NRAS or HRAS Q61K mutations do not introduce a cryptic splice donor site. These findings are consistent with the clinical data showing that the G60G silent mutation occurs uniquely in the KRAS Q61K background (FIG. 2 panel a). Without wishing to be bound by theory, introducing silent mutations exogenously into NRAS Q61K or HRAS Q61K can also render these proteins non-functional. For example, a silent mutation at G60G (A>T or C>T) in NRAS or HRAS Q61K can create a cryptic splice donor site that will induce aberrant splicing (FIG. 3 panel d).

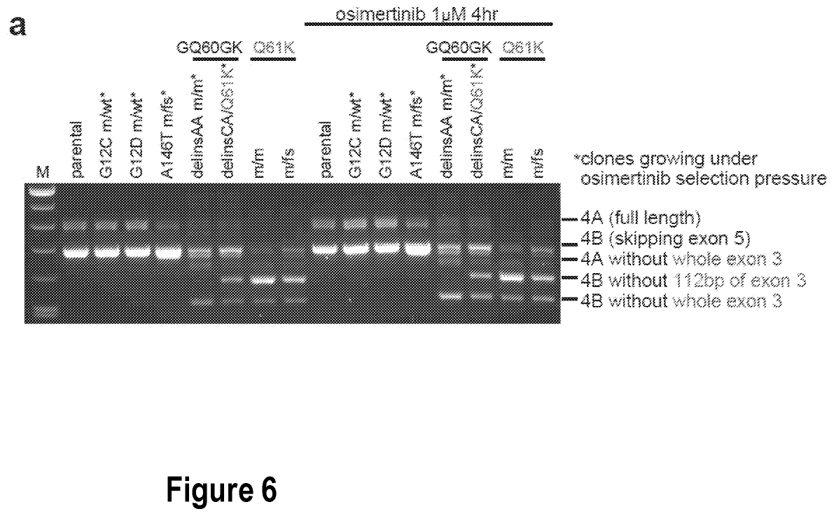

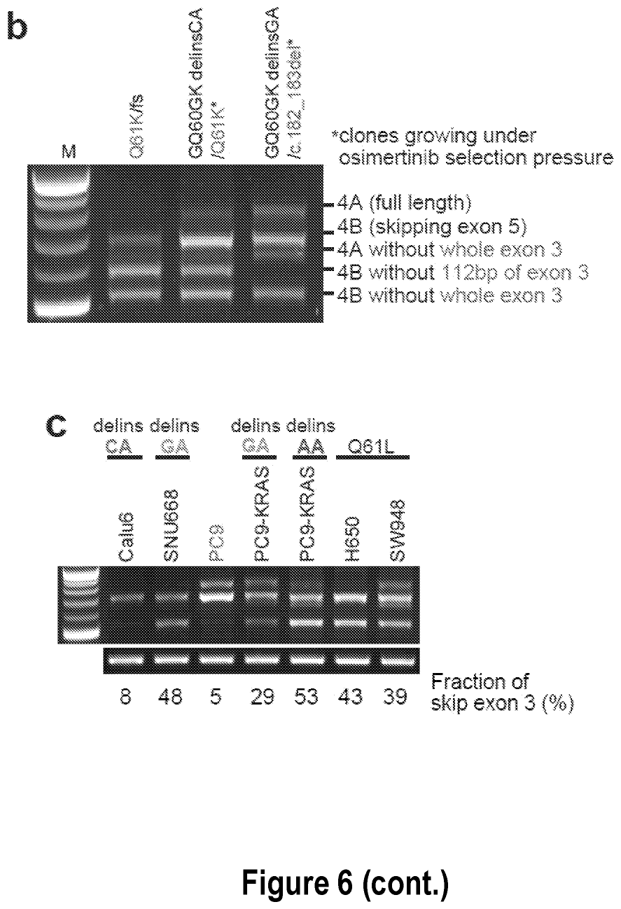

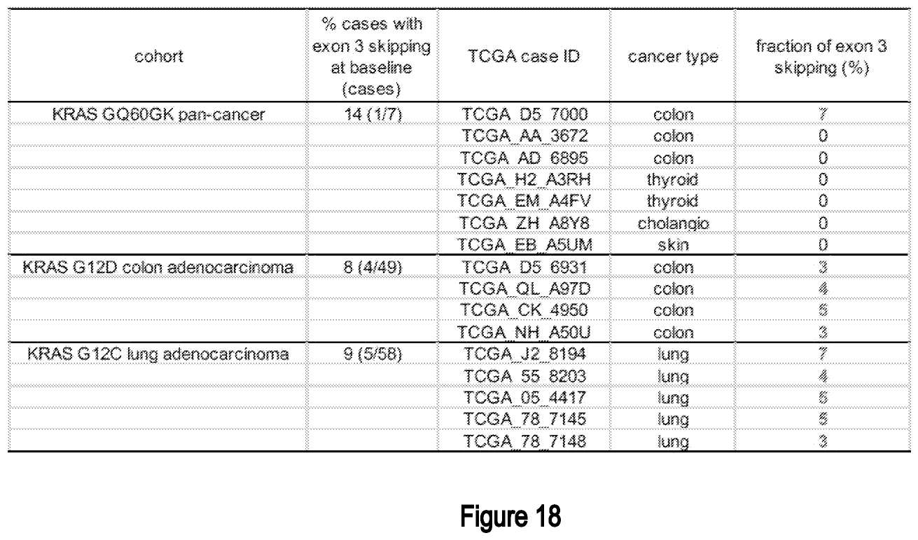

We next validated the mechanism behind skipping of the entire exon 3, leading to premature termination (FIG. 2 panel a pancel c). Using the Human Splicing Finder prediction software²⁴, we surveyed KRAS exon 3 for putative exonic splicing enhancer (ESE) and exonic splicing silencer (ESS) motifs and determined that the region around KRAS Q61K is enriched in ESE motifs (FIG. 3 panel f). ESEs serve as a binding signal for specific serine/arginine-rich (SR) proteins and other splicing regulators which recruit the splicing machinery to weak splice sites flanking an exon and enhance exon inclusion²³. Even a single nucleotide change can disrupt or introduce an ESE or ESS signal and promote aberrant splicing, including one that deletes an entire exon²⁵. A case in point, a 2-nucleotide deletion at KRAS c. 182_183 alters the putative ESE motif dramatically (FIG. 3 panel f), and a clone harboring the c. 182_183del indicates skipping of the entire exon 3 (FIG. 3 panel a). The skipping of the entire exon 3 was also observed in other clones harboring mutations at KRAS Q61 (FIG. 6 panels a, b). A minority of cancers also have a natural proclivity to splice out the entire exon 3 at baseline (FIG. 6 panel c and FIG. 18) although this occurs at a low (10%) frequency. Of note, ESE motifs are also found in NRAS Q61 and HRAS Q61 (FIG. 3 panel f).

Taken together, we uncover a new mechanism, whereby a silent mutation in G60G in the KRAS Q61K context prevents aberrant splicing and can be required to guarantee proper translation of this KRAS mutant. Our screen further exposes other vulnerabilities associated with mutations in the vicinity of KRAS Q61, including fatal alterations to endogenous ESE motifs, leading to aberrant splicing.

### Induction of Aberrant Splicing by Antisense Oligonucleotides

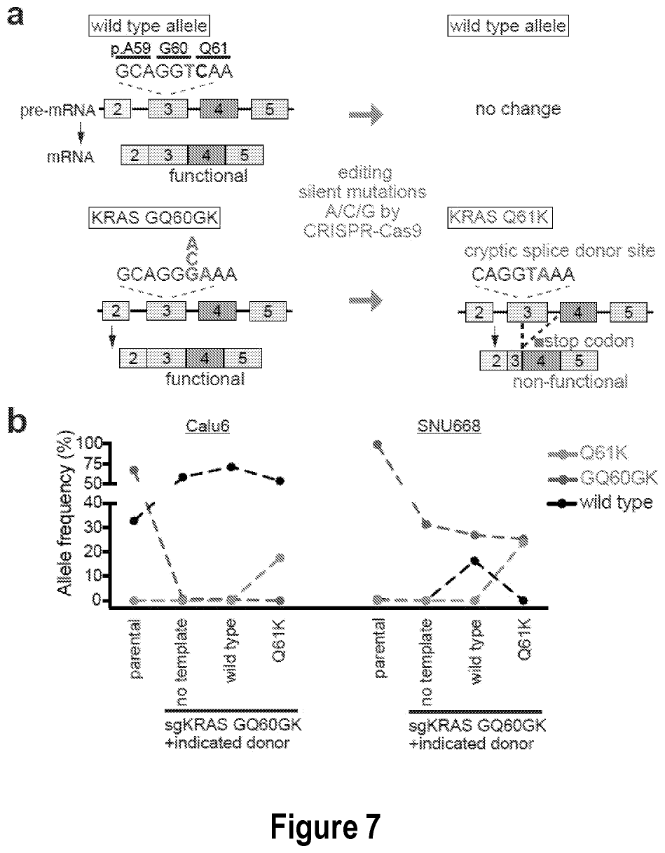

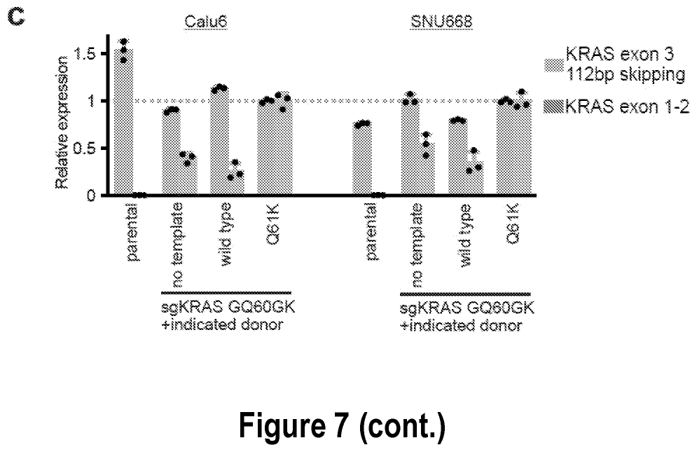

We applied the insights gained from studying the different KRAS mutants that impact splicing into therapeutic strategies. Without wishing to be bound by theory, antisense oligonucleotides designed against the ESE motifs in pre-mRNA of KRAS, NRAS or HRAS, can compete for binding to these sites with the SR proteins, leading to a deleterious exclusion of the whole exon 3 and causing early termination. An alternative strategy is to correct KRAS G60G silent mutations using CRISPR-Cas9 with KRAS mutant-specific sgRNA, in order to convert the original KRAS GQ60GK into the non-functional Q61K (FIG. 7 panels a-c).

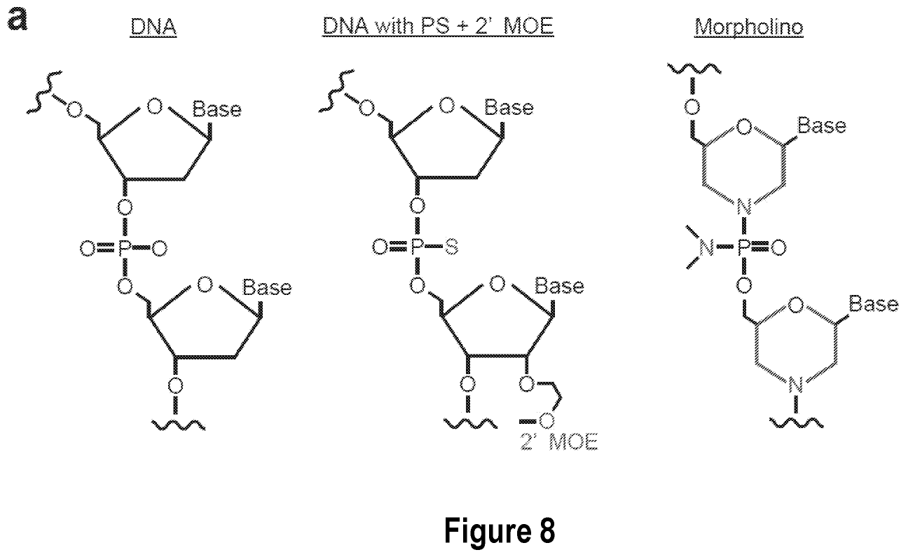

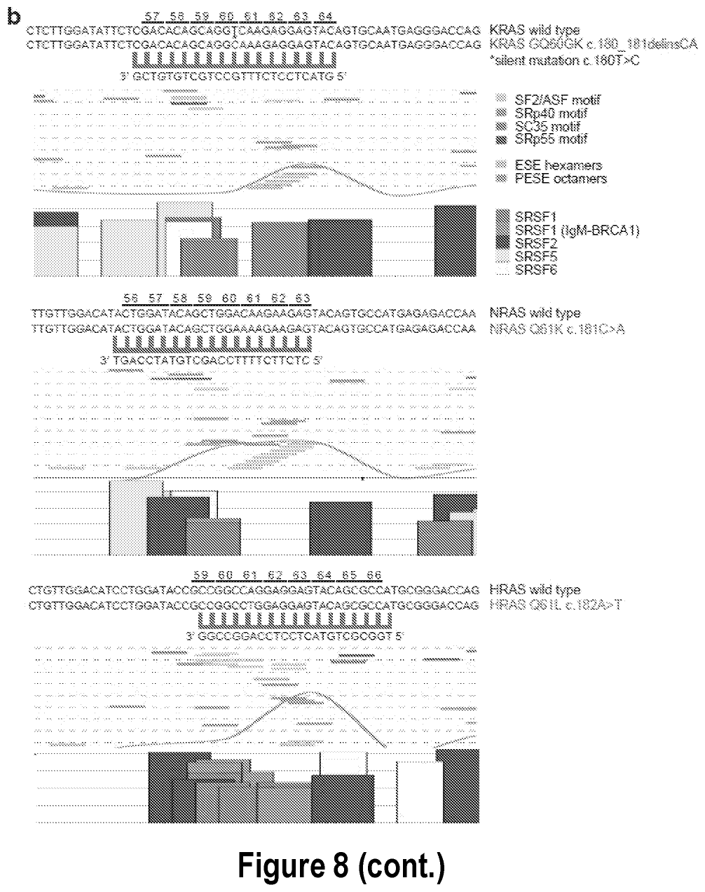

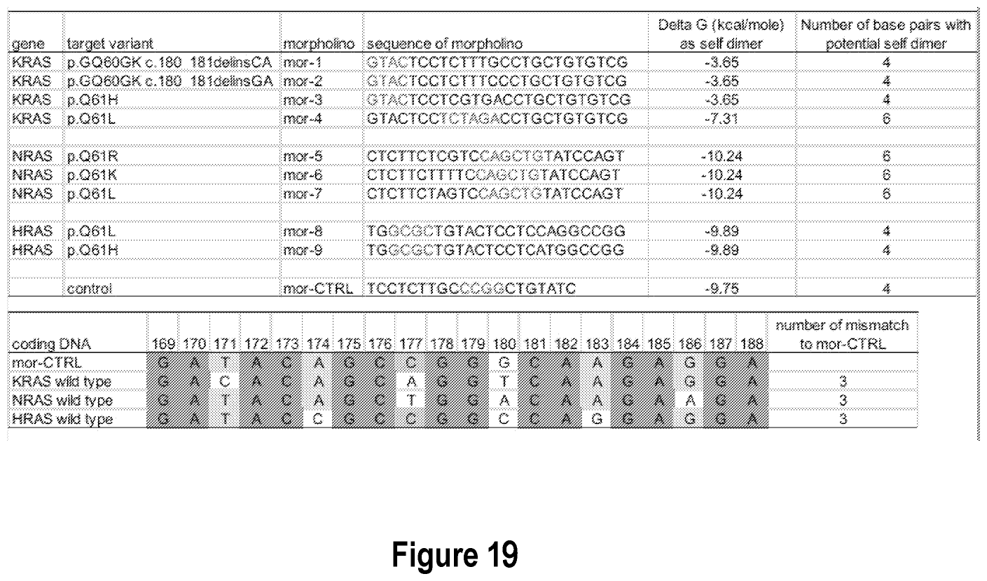

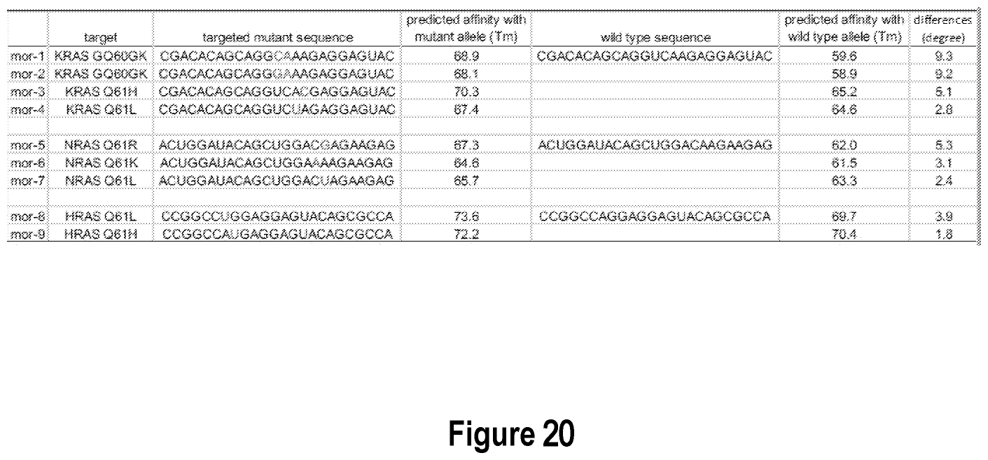

Here, we used morpholino and DNA with phosphorothioate (PS)+2′-O-Methoxyethyl (2′MOE) modifications to generate antisense oligos (FIG. 8 panel a). Both types of antisense oligos are modified from natural nucleic acids to strongly and specifically bind to complementary target sites and are more stable in the presence of nucleases than DNA26. We designed mutant selective morpholinos for KRAS, NRAS, and HRAS Q61X, as well as control morpholinos with 3 mismatches to these sequences, taking into consideration the putative ESE sites and the potential for the oligo to self-dimerize (FIG. 8 panel b and FIG. 19). Morpholino oligos against HRAS Q61X were not able to cover ESEs around codon 57 and 58 as the oligos can self-dimerize and as such not bind this region. The mean difference in predicted affinity (Tm) between morpholinos against the mutant or wild type allele was 4.8 (1.8-9.3) degrees (FIG. 20). Morpholinos targeting KRAS GQ60GK revealed up to a 9.3 degree difference compared to the wild type counterparts.

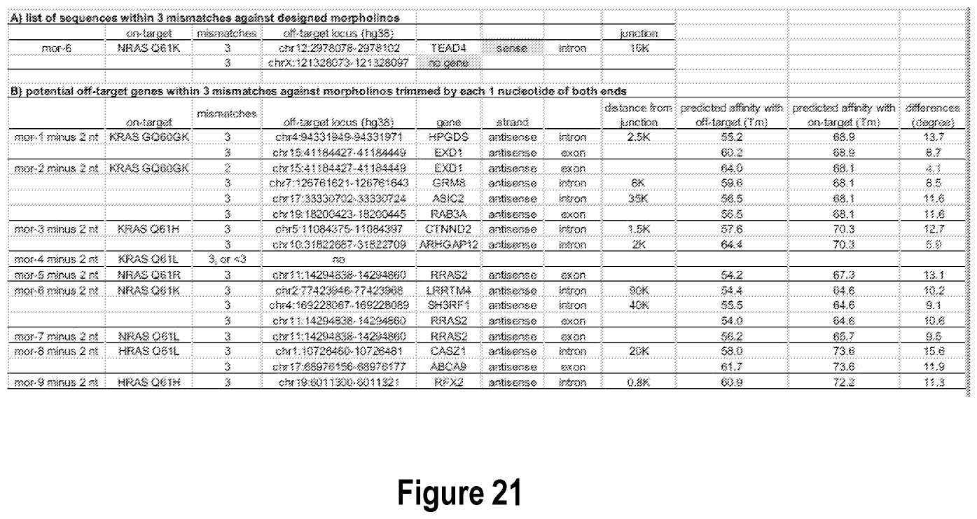

A genome-wide screening of off-target sites of designed morpholinos demonstrated no antisense off-target genes with 100% homology against any of the 9 morpholinos. When allowing for up to 3 mismatches, only mor-6 (3 mismatches) has an off-target homology sequence, but its targets are located on the sense strand (thus, unable to bind) or in non-coding regions (FIG. 21 panel a). Additionally, sequences trimmed by 1 nucleotide on both ends were evaluated to simulate binding of end-degraded morpholinos²⁷. Genes within 3 mismatches showed a mean 10.5 (4.1-15.6) degree difference in the predicted binding affinity between on-target and off-target sites, and as such are unlikely to result in off-target binding (FIG. 21 panel b).

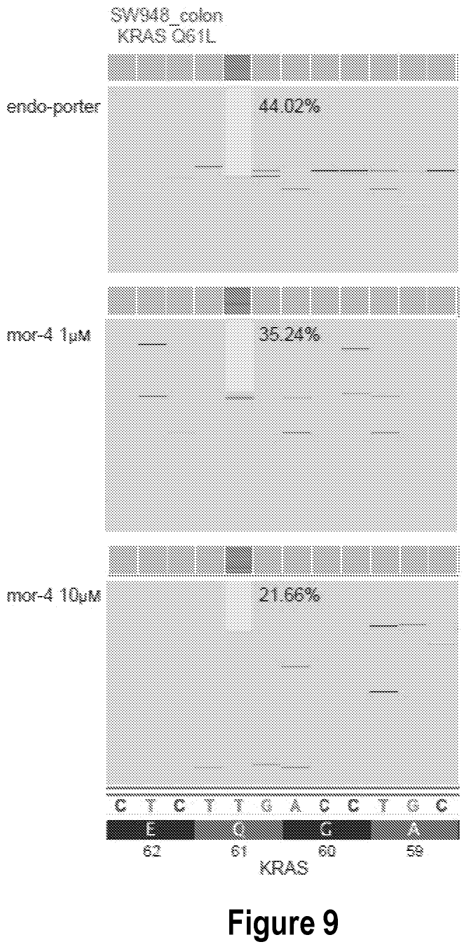

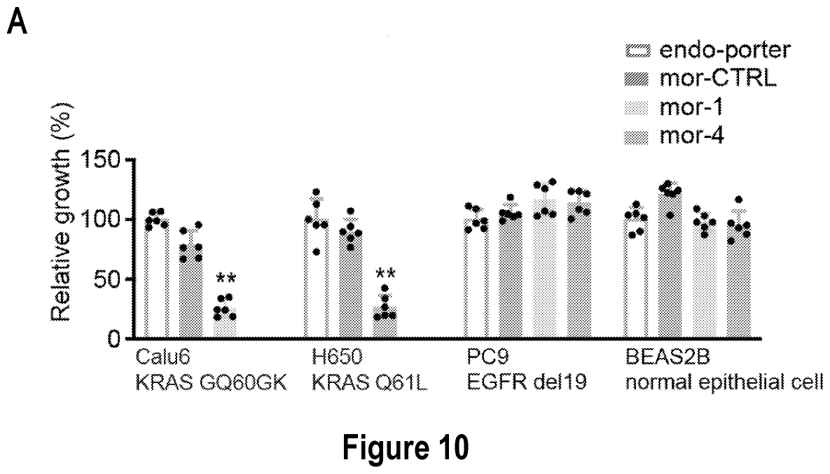

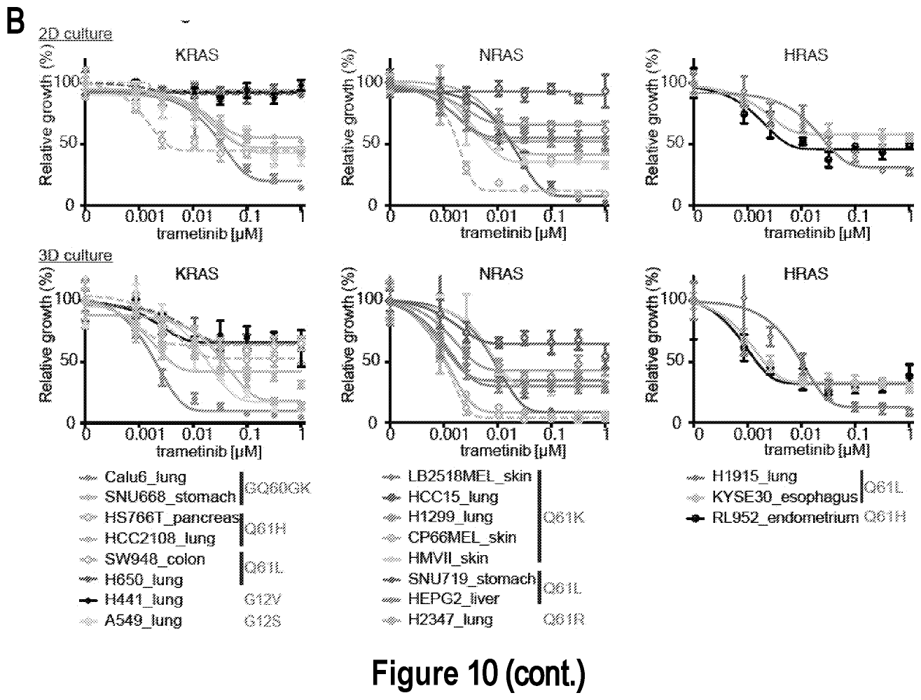

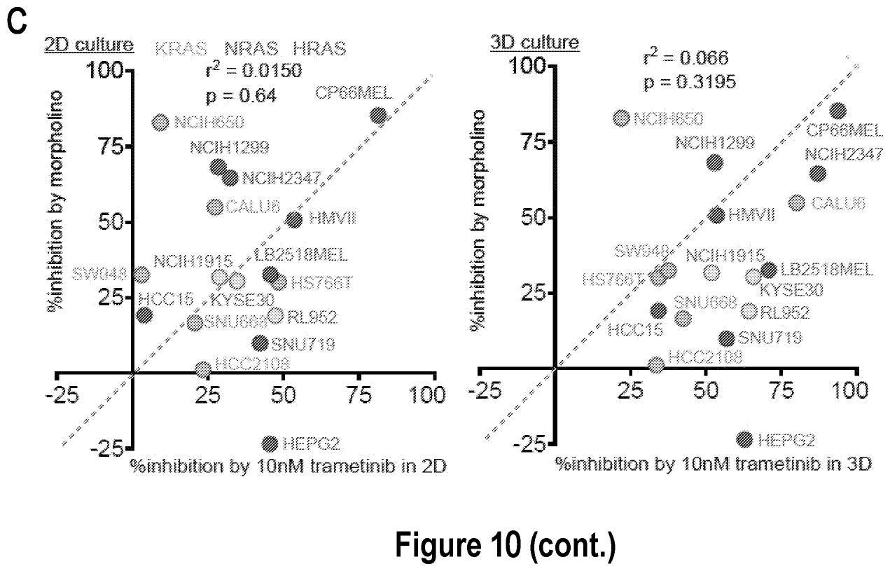

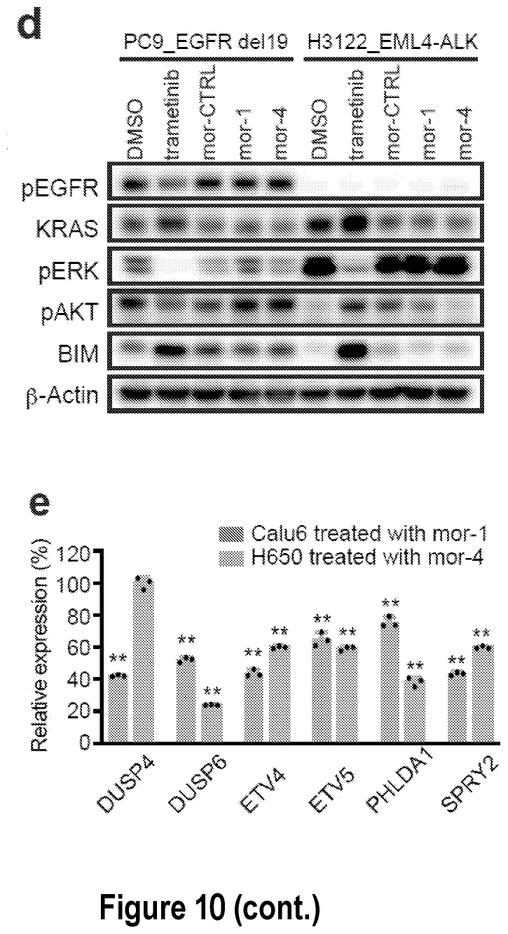

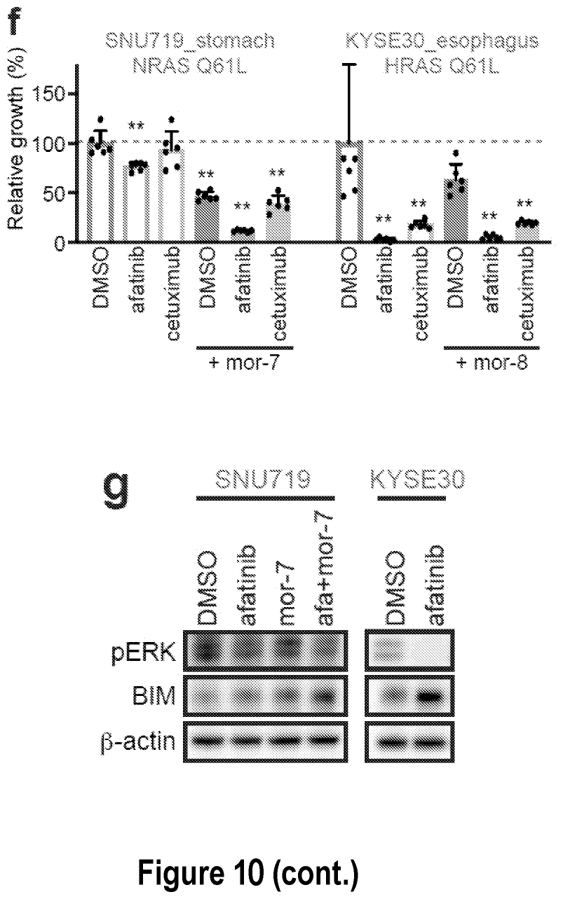

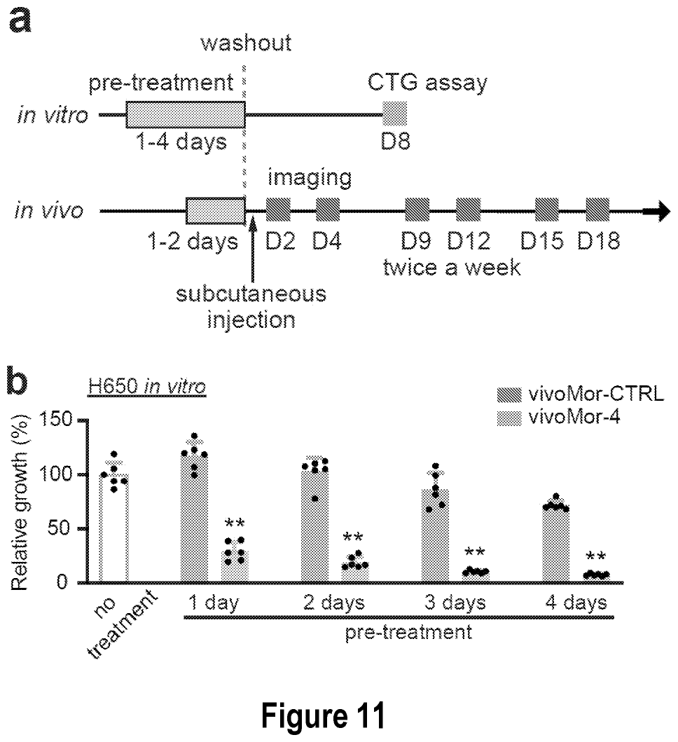

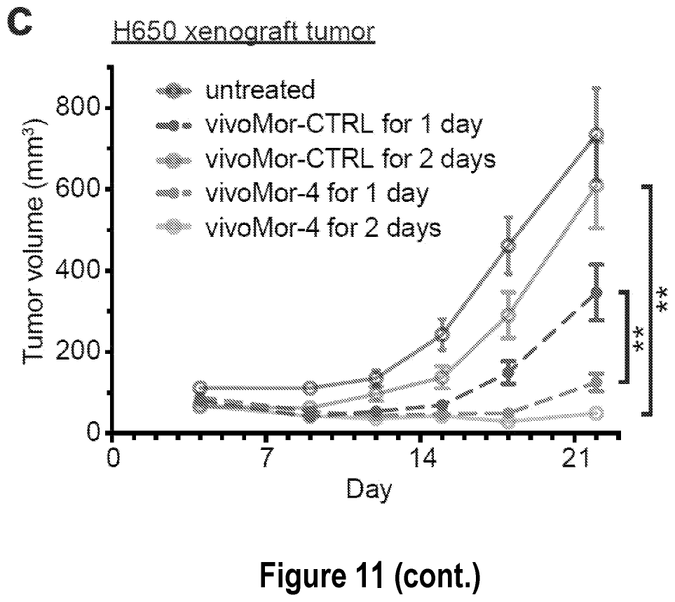

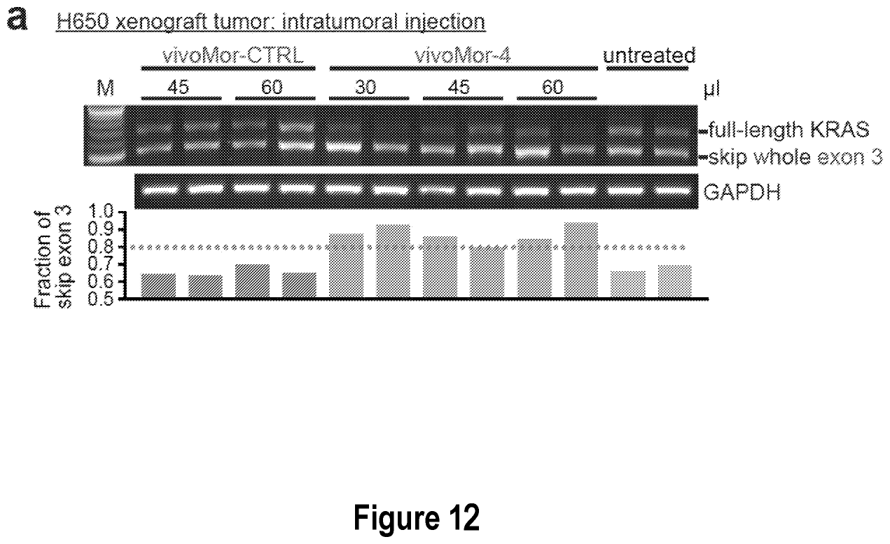

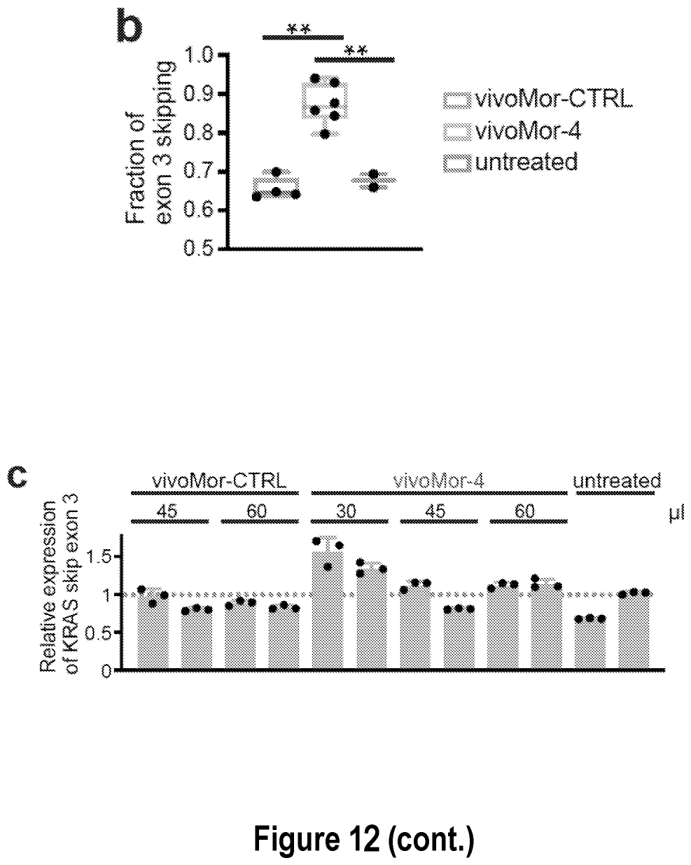

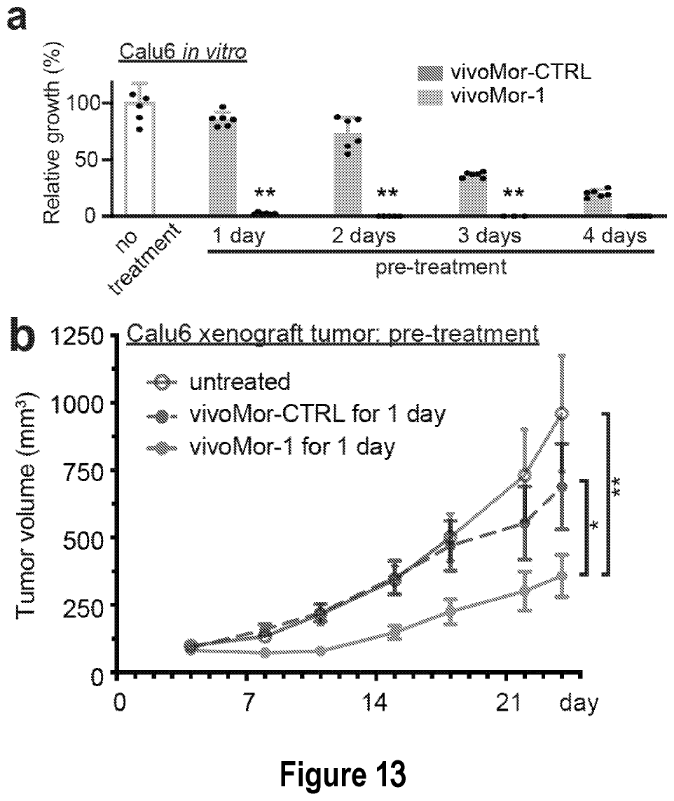

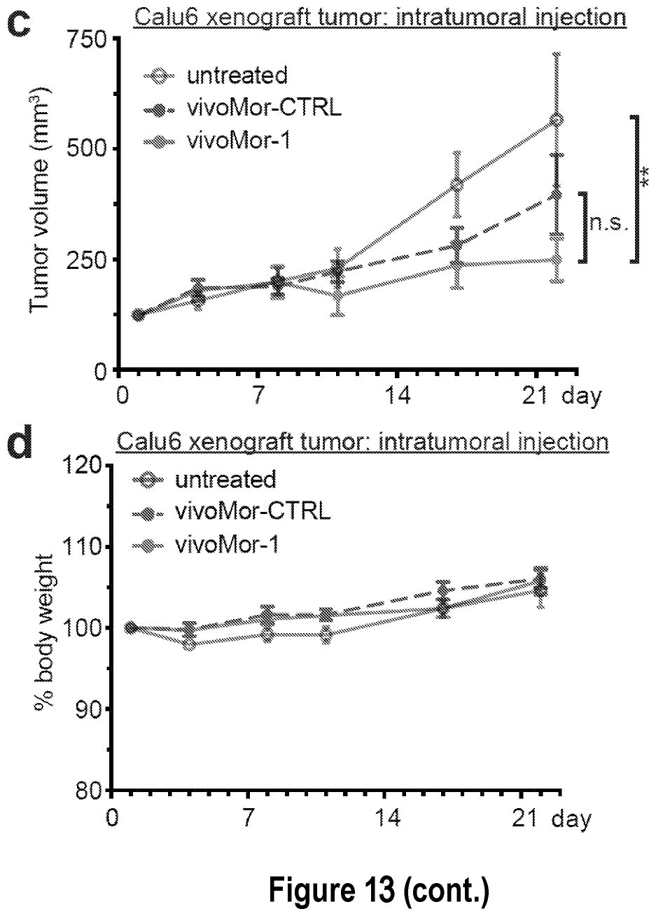

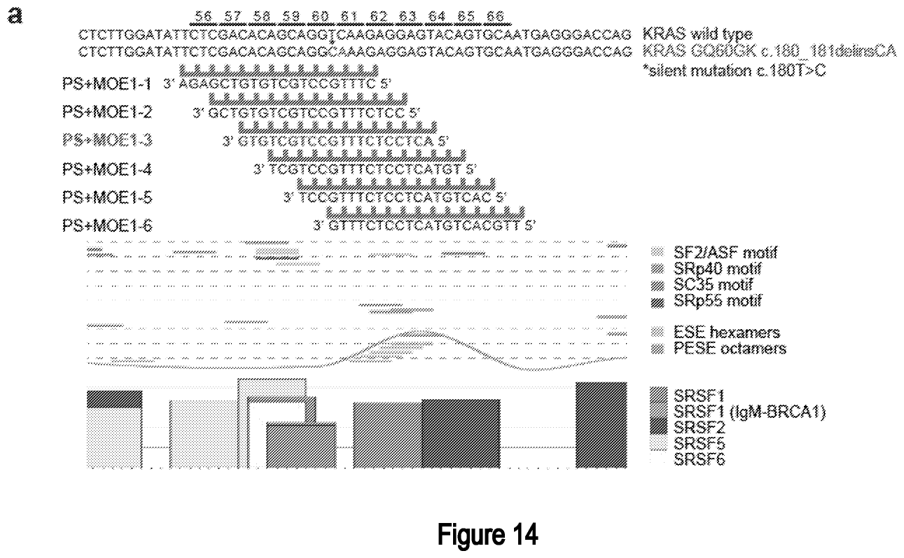

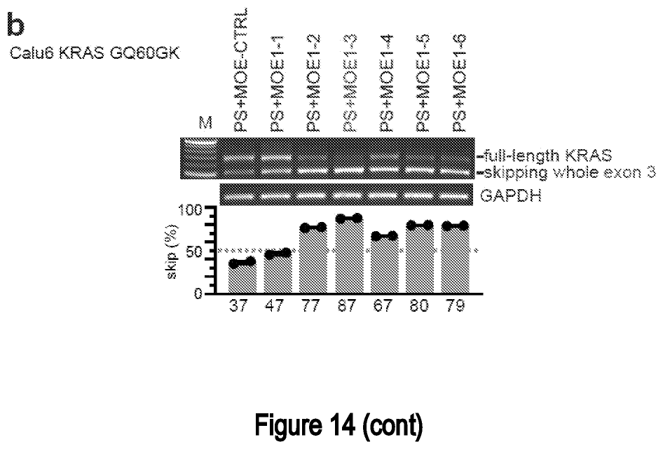

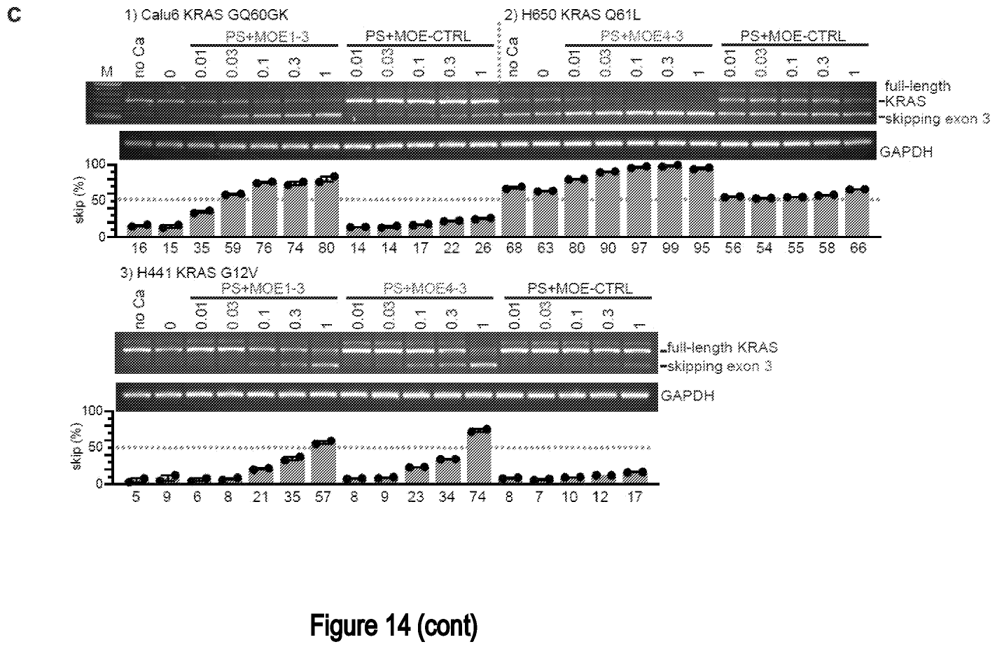

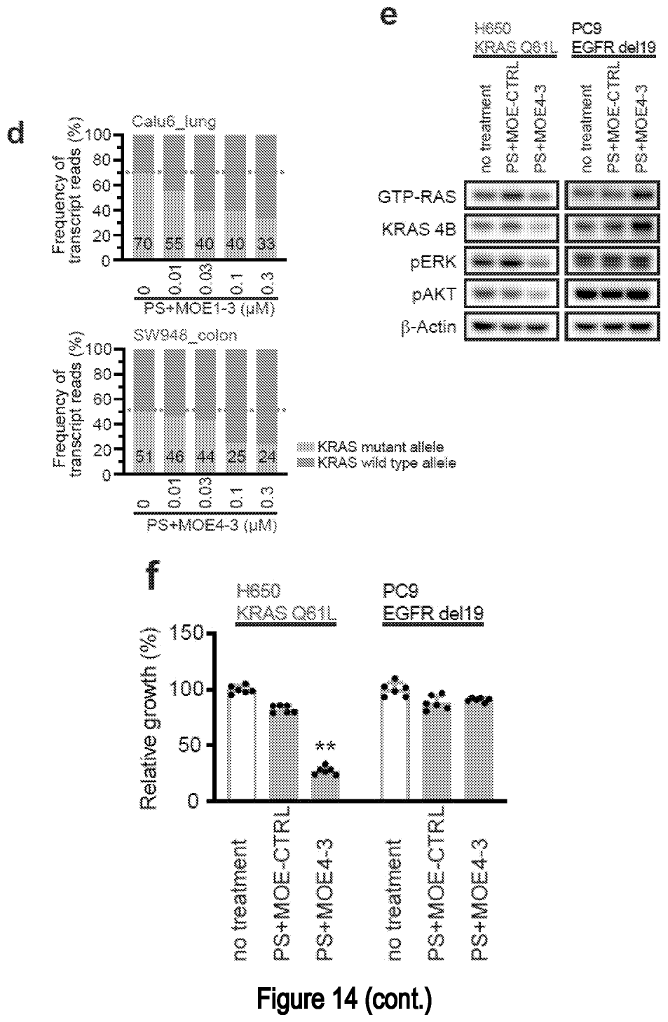

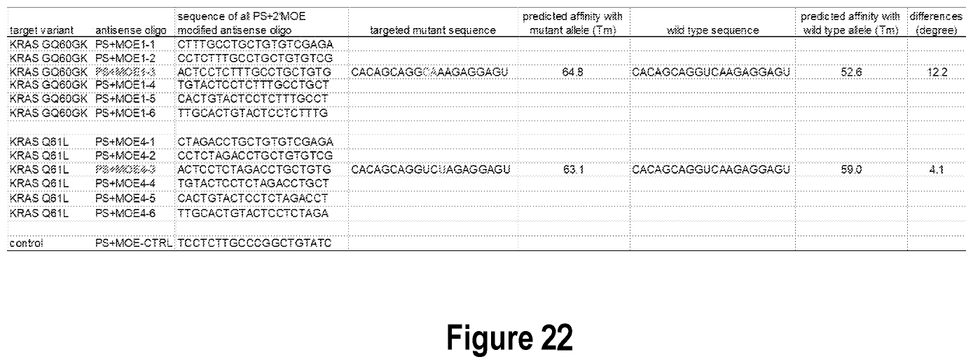

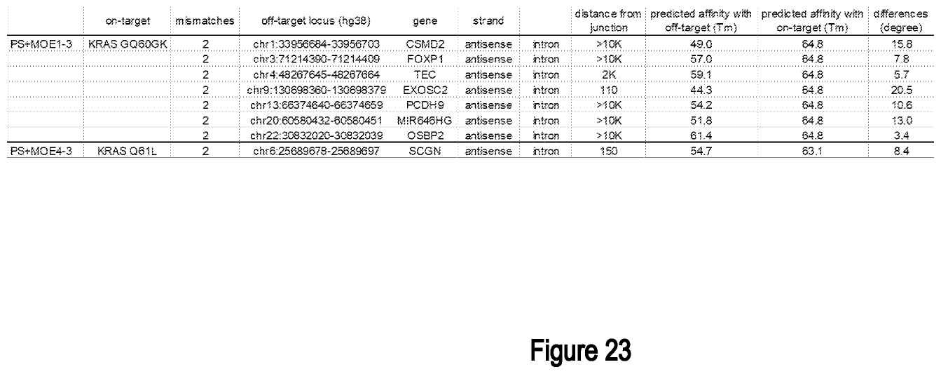

The use of mutant selective antisense oligos can be used to induce aberrant splicing only in tumor cells, but not in normal cells lacking the Q61 mutation, minimizing off target toxicity (FIG. 4 panel a). Using this approach, we induced skipping of whole exon 3 in the KRAS GQ60GK lung cancer CALU6 cell line, in a dose dependent manner (FIG. 4 panel b). No skipping of exon 3 was observed with the control oligo. This observation extended to additional cancer cell lines harboring KRAS, NRAS, and HRAS Q61X mutants demonstrating oligo-mediated mutant selective exon 3 skipping (FIG. 4 panel c). To assess the selectivity of the morpholino for the KRAS mutant over wild type transcript, the identity of the full-length pre-mRNA contributing to the exon 3 spliced mRNA following morpholino treatment needed to be determined. Since this information cannot be extracted from the exon 3 skipped cDNA sequence itself—the Q61 bearing exon 3 is spliced out—we focused our analysis on the composition of the full-length KRAS cDNA amplicon instead. We PCR-amplified KRAS cDNA generated from morpholino treated SW948 cells, separated it by gel electrophoresis and analyzed by NGS the remaining full-length transcript, in order to obtain the ratio of mutant versus wild type KRAS pre-mRNA targeted by the morpholino. NGS analysis analogous to standard AF quantification revealed that mor-4 preferentially targeted the mutant KRAS pre-mRNA and decreased its fraction within the full-length transcript species in a dose dependent manner; from 44% to 22%, which indicates that morpholino treatment induces mutant selective splicing (FIG. 4 panel d and FIG. 9). We next evaluated the effect of these oligos on cell growth. As not all RAS mutant cell lines are dependent on RAS signaling for their growth²⁸, we determined RAS dependency of each of the Q61 mutant cell lines using KRAS, NRAS or HRAS specific siRNAs, and compared our findings to published gene effect scores for dependency by RNAi and CRISPR knock out obtained from Depmap (depmap.org) (FIG. 4 panel e). Cell lines that were RAS dependent for their growth as determined by siRNA were also growth inhibited following selective morpholino oligo treatment (FIG. 4 panel e and FIG. 10 panel a). In aggregate, there was a significant concordance between growth inhibition by siRNA and morpholino oligo treatment (FIG. 4 panel f). We also evaluated MEK inhibitor sensitivity in a panel of these cell lines using trametinib (FIG. 10 panel b). Of note, the KRAS Q61L H650 lung cell line is highly resistant to trametinib but sensitive to antisense oligo (FIG. 10 panels b, c). Resistance to trametinib in H650 is the result of pEGFR reactivation and sustained pAKT signals, whereas antisense oligo treatment inhibited both pERK and pAKT without this feedback (FIG. 4 panel g, and FIG. 10 panel d). We further confirmed that the expression of ERK signature genes²⁹ in KRAS mutant cells was reduced after treatment with antisense oligos (FIG. 10 panel e). In models not sensitive to single agent morpholino oligo treatment, some were sensitive to EGFR inhibitor monotherapy or to the combination of an EGFR inhibitor and the morpholino (FIG. 10 panels f, g). We next studied the morpholino treatment strategy in vivo. We employed a pre-treatment strategy (FIG. 11 panel a). When piloted in vitro, morpholino oligo pre-treatment for 1 to 4 days followed by wash-out potently inhibited cell growth (FIG. 11 panel b). We subsequently pre-treated H650 lung cancer cells for 1 or 2 days with either the mutant-selective or control vivo-morpholino before injecting the same number of viable cells from each treatment condition in vivo. The control vivo-morpholino treated cells grew at a comparable rate to the untreated cells (FIG. 4 panels h, i). However, there was a significant decrease in growth in vivo as measured by either luciferase or tumor volume in the cells treated with the mutant-selective morpholino (FIG. 4 panels h, i and FIG. 11 panel c). Furthermore, intra-tumoral injection of vivo-morpholinos demonstrated induction of KRAS exon 3 skipping (FIG. 12 panels a-d), and a significant reduction in tumor size compared to CTRL or untreated tumors (FIG. 4 panel j). In a second KRAS GQ60GK mutant xenograft model (Calu6), treatment was also effective when the pre-treatment approach was used (FIG. 13 panels a, b), and led to a positive efficacy trend through the intra-tumoral injection approach (FIG. 13 panels c, d). We further evaluated the efficacy of a second type of antisense oligo technology, PS+2′MOE oligos and observed that they achieved mutant selective exon skipping and growth inhibition in vitro at much lower concentrations compared with morpholino oligos (FIG. 14 panels a-f and FIGS. 22 and 23).

### Discussion

We uncover a role of silent mutations in splicing and production of a functional oncogene. Our RAS-directed CRISPR editing and drug pressure screen shed light on the effects of silent mutations, namely KRAS G60G, on splicing and translation of a functional KRAS Q61K. Prior studies using conventional extrinsic overexpression of the coding sequences alone would not have identified the biological necessity of the silent mutation because the cDNA of an already spliced transcript is employed in such models. The CRISPR-models, which enable the evaluation of splicing events, as well as manual review of KRAS G60G silent mutations in clinical samples uncovered new biology of KRAS G60G which had not been known. A functional KRAS Q61K requires a dinucleotide change and as such can explain the rarity of this mutation in patients (0.7% of all KRAS mutations) in contrast to NRAS Q61K (20% of all NRAS mutations) or HRAS Q61K (7% of all HRAS) mutations, which are oncogenic due to a single base pair substitution. The functional significance of KRAS G60G silent mutations have been unknown³⁰. We identified two different splicing vulnerabilities that can be exploited therapeutically: a cryptic splice donor site in KRAS GQ60GK cancers, and ESE motifs in KRAS, NRAS and HRAS Q61X mutant cancers. The results indicate that the induction of aberrant exon 3 exclusion in a mutant selective manner using an antisense oligonucleotide approach produces nonfunctional RAS mutant protein and leads to tumor cell growth inhibition in vitro and in vivo. Our findings indicate the therapeutic utility of direct inhibition of RAS Q61X cancers.

Treatments using splice modulating morpholinos for Duchenne muscular dystrophy and antisense oligo with PS+2′MOE for spinal muscular atrophy are FDA approved therapies, indicating clinical feasibility of our RAS Q61X directed antisense oligo approach. Unlike prior anti-sense strategies targeting STAT3 or KRAS, our strategy for targeting RAS Q61X is mutant selective and can result in a wider therapeutic index and less toxicity in normal tissues^{31,32}. As not all KRAS mutant tumors are dependent on RAS but also other signals including EGFR33 and targeting KRAS G12C achieved responses in only a subset¹⁵, the correlation between our morpholino's efficacy and RAS dependency further support the on-target effects of our strategy. Embodiments of in vivo delivery of antisense oligos can include chemical modifications, including conjugation to cell penetrating short peptides^{34,35}, encapsulation, and viral delivery³⁶.

Our findings provide new insights into the biological role of silent mutations in oncogenes and their ability to be translated into new therapies. The applicability of this strategy can extend to other genes based on comprehensive analyses of silent mutations³⁷. Further development of DNA editing technologies can enable direct editing of KRAS G60G silent mutations to induce aberrant splicing in KRAS Q61K cancers, abolishing their oncogenic capacity.

### REFERENCES CITED IN THIS EXAMPLE

1 Middleton, G. et al. The National Lung Matrix Trial of personalized therapy in lung cancer. Nature 583, 807-812, doi: 10.1038/s41586-020-2481-8 (2020).
2 Ostrem, J. M., Peters, U., Sos, M. L., Wells, J. A. & Shokat, K. M. K-Ras(G12C) inhibitors allosterically control GTP affinity and effector interactions. Nature 503, 548-551, doi:10.1038/nature12796 (2013).
3 Canon, J. et al. The clinical KRAS(G12C) inhibitor AMG 510 drives anti-tumour immunity. Nature 575, 217-223, doi: 10.1038/s41586-019-1694-1 (2019).
4 Hallin, J. et al. The KRAS(G12C) Inhibitor MRTX849 Provides Insight toward Therapeutic Susceptibility of KRAS-Mutant Cancers in Mouse Models and Patients. Cancer Discov 10, 54-71, doi: 10.1158/2159-8290.CD-19-1167 (2020).
5 Zehir, A. et al. Mutational landscape of metastatic cancer revealed from prospective clinical sequencing of 10,000 patients. Nat Med 23, 703-713, doi: 10.1038/nm.4333 (2017).
6 Ramalingam, S. S. et al. Overall Survival with Osimertinib in Untreated, EGFR-Mutated Advanced NSCLC. N Engl J Med 382, 41-50, doi: 10.1056/NEJMoa1913662 (2020).
7 Diederichs, S. et al. The dark matter of the cancer genome: aberrations in regulatory elements, untranslated regions, splice sites, non-coding RNA and synonymous mutations. EMBO Mol Med 8, 442-457, doi: 10.15252/emmm.201506055 (2016).
8 Group, P. T. C. et al. Genomic basis for RNA alterations in cancer. Nature 578, 129-136, doi: 10.1038/s41586-020-1970-0 (2020).
9 Consortium, A. P. G. AACR Project GENIE: Powering Precision Medicine through an International Consortium. Cancer Discov 7, 818-831, doi: 10.1158/2159-8290.CD-17-0151 (2017).
10 Janne, P. A. et al. Selumetinib Plus Docetaxel Compared With Docetaxel Alone and Progression-Free Survival in Patients With KRAS-Mutant Advanced Non-Small Cell Lung Cancer: The SELECT-1 Randomized Clinical Trial. JAMA 317, 1844-1853, doi:10.1001/jama.2017.3438 (2017).
11 Kitai, H. et al. Epithelial-to-Mesenchymal Transition Defines Feedback Activation of Receptor Tyrosine Kinase Signaling Induced by MEK Inhibition in KRAS-Mutant Lung Cancer. Cancer Discov 6, 754-769, doi: 10.1158/2159-8290.CD-15-1377 (2016).
12 Kruspig, B. et al. The ERBB network facilitates KRAS-driven lung tumorigenesis. Sci Transl Med 10, doi: 10.1126/scitranslmed.aao2565 (2018).
13 Moll, H. P. et al. Afatinib restrains K-RAS-driven lung tumorigenesis. Sci Transl Med 10, doi: 10.1126/scitranslmed.aao2301 (2018).
14 LaMarche, M. J. et al. Identification of TNO155, an Allosteric SHP2 Inhibitor for the Treatment of Cancer. J Med Chem, doi:10.1021/acs.jmedchem.0c01170 (2020).
15 Hong, D. S. et al. KRAS(G12C) Inhibition with Sotorasib in Advanced Solid Tumors. N Engl J Med, doi: 10.1056/NEJMoa1917239 (2020).
16 Hunter, J. C. et al. Biochemical and Structural Analysis of Common Cancer-Associated KRAS Mutations. Mol Cancer Res 13, 1325-1335, doi: 10.1158/1541-7786.MCR-15-0203 (2015).
17 Zhou, Z. W. et al. KRASQ61H preferentially signals through MAPK in a RAF dimer-dependent manner in non-small cell lung cancer. Cancer Res, doi: 10.1158/0008-5472.CAN-20-0448 (2020).
18 Oxnard, G. R. et al. Assessment of Resistance Mechanisms and Clinical Implications in Patients With EGFR T790M-Positive Lung Cancer and Acquired Resistance to Osimertinib. JAMA Oncol, doi: 10.1001/jamaoncol.2018.2969 (2018).
19 Ramalingam, S. S. et al. Mechanisms of acquired resistance to first-line osimertinib: Preliminary data from the phase III FLAURA study. Annals of Oncology 29, doi:10.1093/annonc/mdy424.063 (2018).
20 Reinert, T. et al. Analysis of Plasma Cell-Free DNA by Ultradeep Sequencing in Patients With Stages I to III Colorectal Cancer. JAMA Oncol, doi:10.1001/jamaoncol.2019.0528 (2019).
21 Chabon, J. J. et al. Integrating genomic features for non-invasive early lung cancer detection. Nature 580, 245-251, doi: 10.1038/s41586-020-2140-0 (2020).
22 Amendola, C. R. et al. KRAS4A directly regulates hexokinase 1. Nature 576, 482-486, doi: 10.1038/s41586-019-1832-9 (2019).
23 Cartegni, L., Chew, S. L. & Krainer, A. R. Listening to silence and understanding nonsense: exonic mutations that affect splicing. Nat Rev Genet 3, 285-298, doi: 10.1038/nrg775 (2002).
24 Desmet, F. O. et al. Human Splicing Finder: an online bioinformatics tool to predict splicing signals. Nucleic Acids Res 37, e67, doi: 10.1093/nar/gkp215 (2009).
25 McVety, S., Li, L., Gordon, P. H., Chong, G. & Foulkes, W. D. Disruption of an exon splicing enhancer in exon 3 of MLH1 is the cause of HNPCC in a Quebec family. J Med Genet 43, 153-156, doi: 10.1136/jmg.2005.031997 (2006).
26 Khvorova, A. & Watts, J. K. The chemical evolution of oligonucleotide therapies of clinical utility. Nat Biotechnol 35, 238-248, doi: 10.1038/nbt.3765 (2017).
27 Kim, J. et al. Patient-Customized Oligonucleotide Therapy for a Rare Genetic Disease. N Engl J Med 381, 1644-1652, doi: 10.1056/NEJMoa1813279 (2019).
28 Janes, M. R. et al. Targeting KRAS Mutant Cancers with a Covalent G12C-Specific Inhibitor. Cell 172, 578-589 e517, doi: 10.1016/j.cell.2018.01.006 (2018).
29 Brant, R. et al. Clinically Viable Gene Expression Assays with Potential for Predicting Benefit from MEK Inhibitors. Clin Cancer Res 23, 1471-1480, doi:10.1158/1078-0432.CCR-16-0021 (2017).
30 Chang, M. T. et al. Identifying recurrent mutations in cancer reveals widespread lineage diversity and mutational specificity. Nat Biotechnol 34, 155-163, doi:10.1038/nbt.3391 (2016).
31 Zammarchi, F. et al. Antitumorigenic potential of STAT3 alternative splicing modulation. Proc Natl Acad Sci USA 108, 17779-17784, doi: 10.1073/pnas.1108482108 (2011).
32 Ross, S. J. et al. Targeting KRAS-dependent tumors with AZD4785, a high-affinity therapeutic antisense oligonucleotide inhibitor of KRAS. Sci Transl Med 9, doi:10.1126/scitranslmed.aal5253 (2017).
33 Amodio, V. et al. EGFR blockade reverts resistance to KRAS G12C inhibition in colorectal cancer. Cancer Discov, doi: 10.1158/2159-8290.CD-20-0187 (2020).
34 Klein, A. F. et al. Peptide-conjugated oligonucleotides evoke long-lasting myotonic dystrophy correction in patient-derived cells and mice. J Clin Invest 129, 4739-4744, doi: 10.1172/JCI128205 (2019).
35 Boisguerin, P. et al. Delivery of therapeutic oligonucleotides with cell penetrating peptides. Adv Drug Deliv Rev 87, 52-67, doi: 10.1016/j.addr.2015.02.008 (2015).
36 Imbert, M., Dias-Florencio, G. & Goyenvalle, A. Viral Vector-Mediated Antisense Therapy for Genetic Diseases. Genes (Basel) 8, doi: 10.3390/genes8020051 (2017).
37 Sharma, Y. et al. A pan-cancer analysis of synonymous mutations. Nat Commun 10, 2569, doi: 10.1038/s41467-019-10489-2 (2019).
38 Cartegni, L., Wang, J., Zhu, Z., Zhang, M. Q. & Krainer, A. R. ESEfinder: A web resource to identify exonic splicing enhancers. Nucleic Acids Res 31, 3568-3571, doi:10.1093/nar/gkg616 (2003).
39 Smith, P. J. et al. An increased specificity score matrix for the prediction of SF2/ASF-specific exonic splicing enhancers. Hum Mol Genet 15, 2490-2508, doi:10.1093/hmg/ddl171 (2006).
40 Fairbrother, W. G. et al. RESCUE-ESE identifies candidate exonic splicing enhancers in vertebrate exons. Nucleic Acids Res 32, W187-190, doi:10.1093/nar/gkh393 (2004).
41 Zhang, X. H. & Chasin, L. A. Computational definition of sequence motifs governing constitutive exon splicing. Genes Dev 18, 1241-1250, doi: 10.1101/gad. 1195304 (2004).
42 Hori, S.-i. et al. Ca2+ enrichment in culture medium potentiates effect of oligonucleotides. Nucleic Acids Research 43, e128-e128, doi: 10.1093/nar/gkv626 (2015).
43 Garcia, E. P. et al. Validation of OncoPanel: A Targeted Next-Generation Sequencing Assay for the Detection of Somatic Variants in Cancer. Arch Pathol Lab Med 141, 751-758, doi: 10.5858/arpa.2016-0527-OA (2017).
44 Odegaard, J. I. et al. Validation of a Plasma-Based Comprehensive Cancer Genotyping Assay Utilizing Orthogonal Tissue- and Plasma-Based Methodologies. Clin Cancer Res 24, 3539-3549, doi: 10.1158/1078-0432.CCR-17-3831 (2018).

### Methods

### Cell Lines and Drugs

Information on cell lines are listed in supplementary table 10. All cell lines were periodically tested for Mycoplasma using the Mycoplasma Plus PCR Primer Set (Agilent) throughout the study. Osimertinib, trametinib, and afatinib were purchased from Selleck Chemicals. Cetuximab was purchased from Dana Farber Cancer Institute pharmacy.

### Animals

7-week old female NSG mice (for H650 xenograft model) and NCr nude mice (for Calu-6 xenograft model) were purchased from The Jackson Laboratory. Animals were allowed to acclimate for at least 5 days before initiation of the study. All in vivo studies were conducted at Dana-Farber Cancer Institute with the approval of the Institutional Animal Care and Use Committee in an AAALAC accredited vivarium.

### Genome Editing Using CRISPR-Cas9

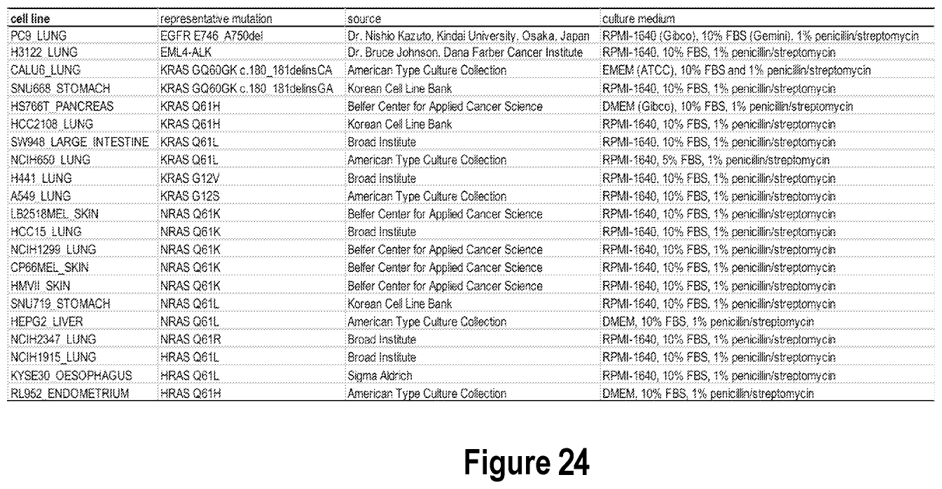

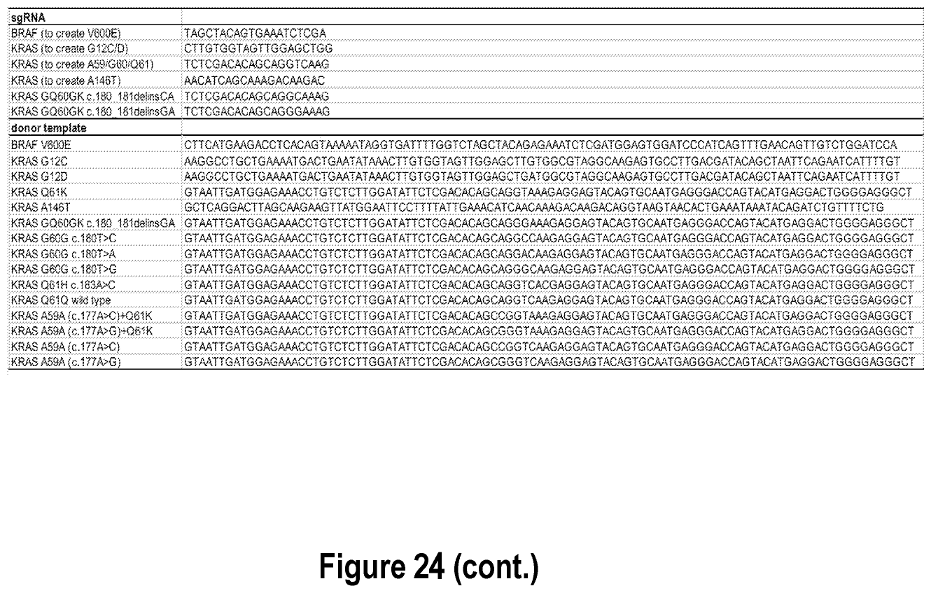

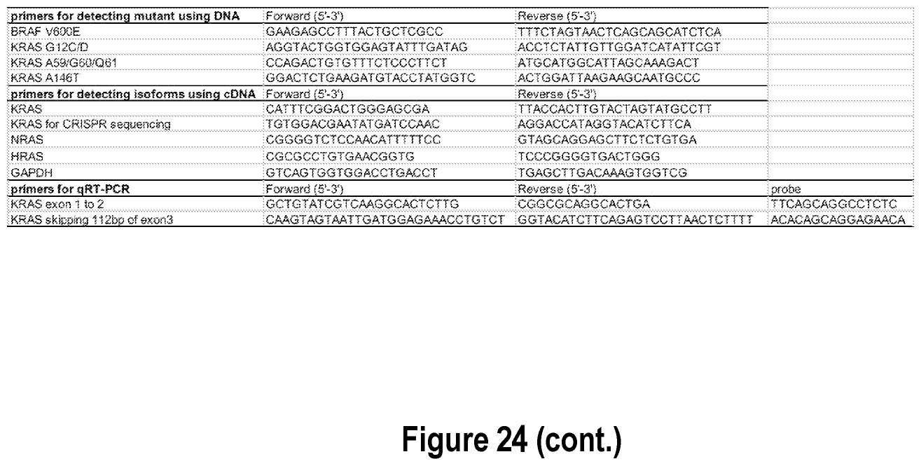

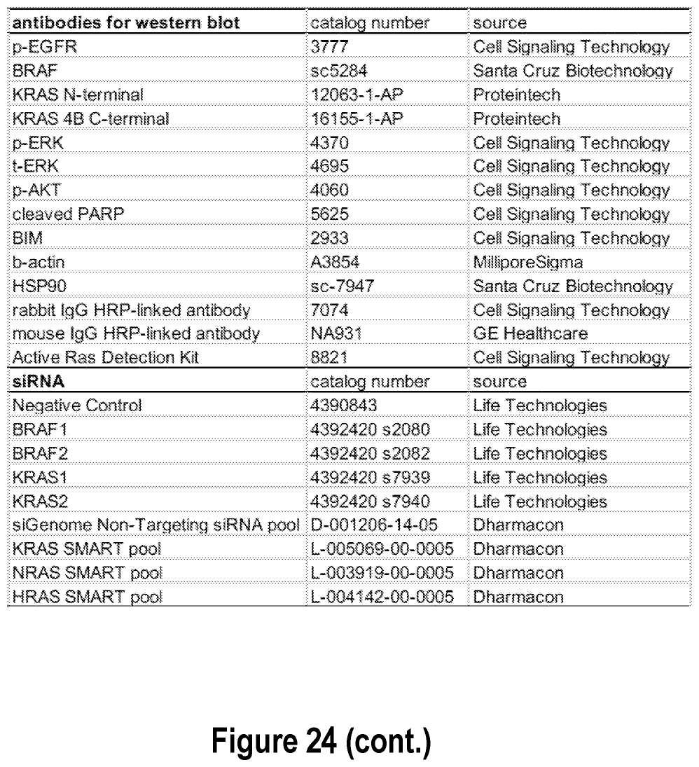

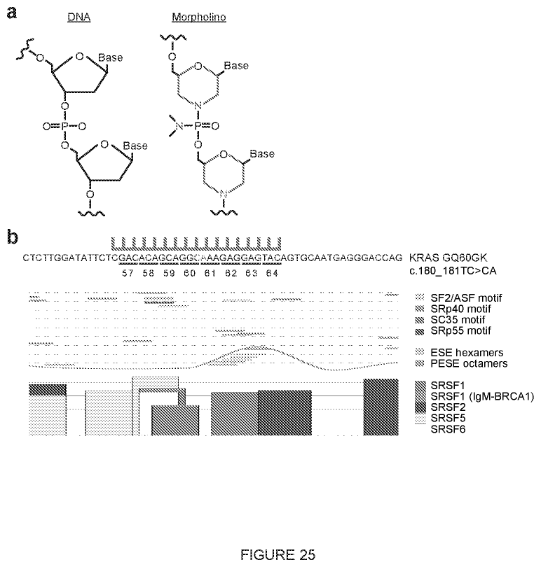

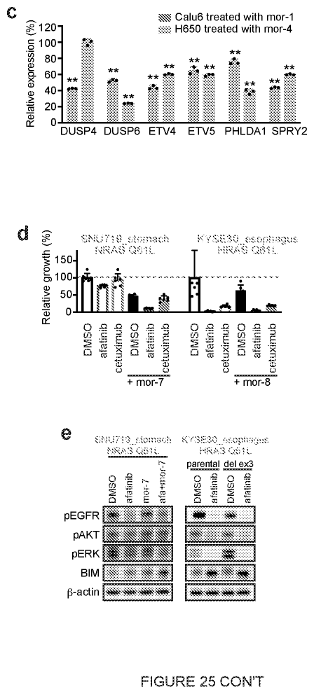

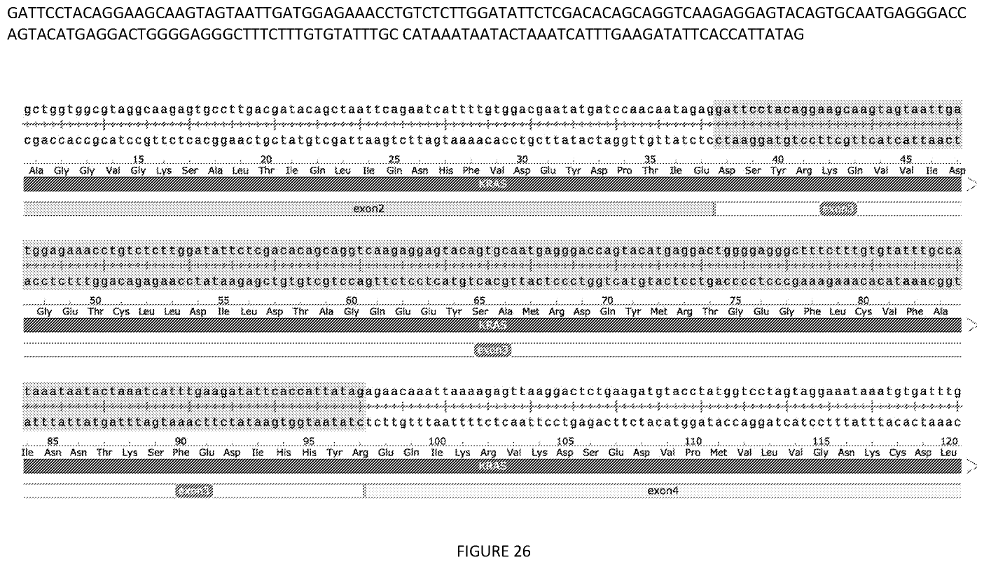

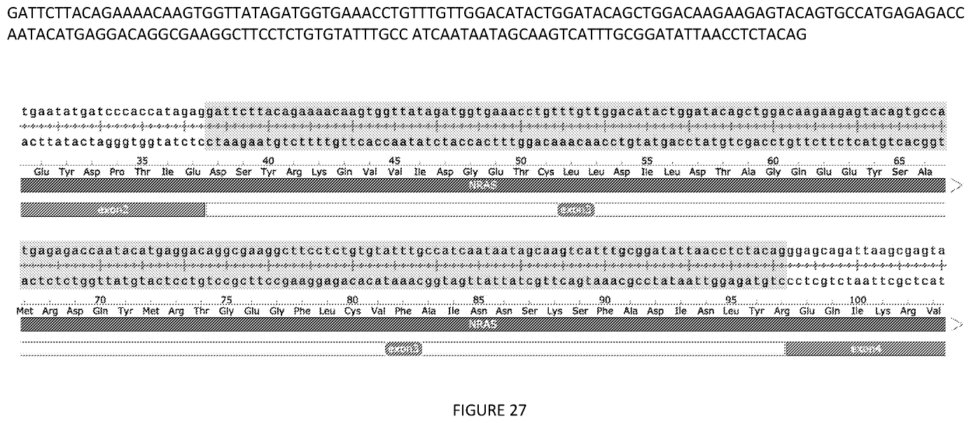

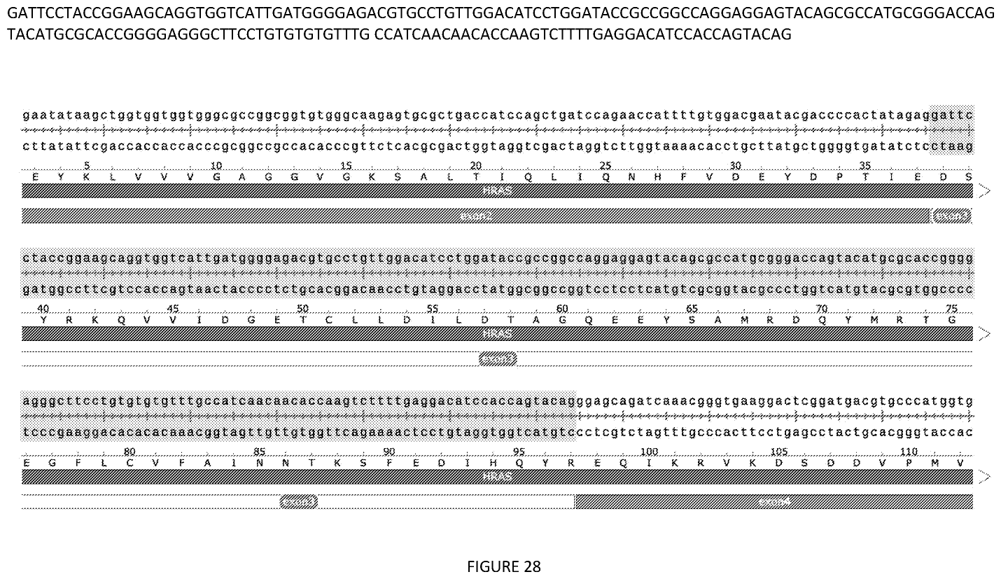

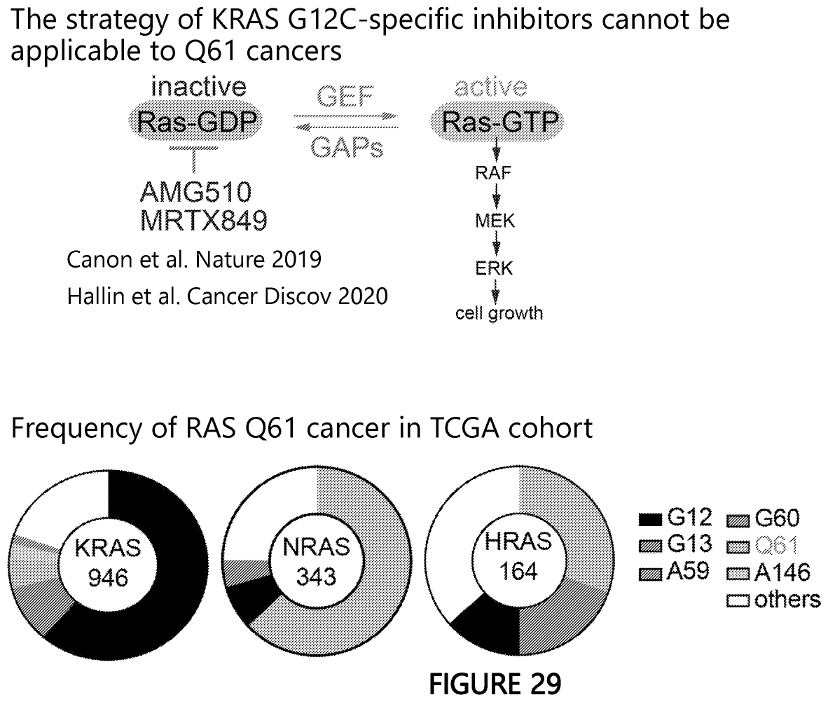

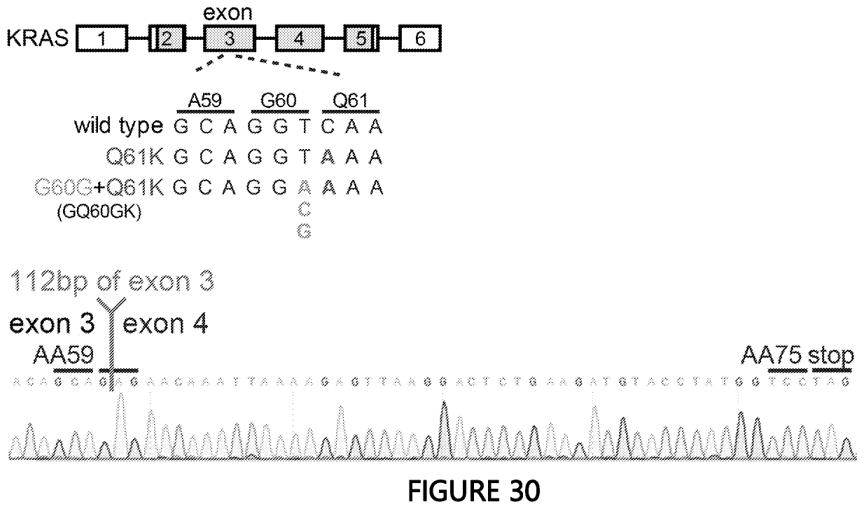

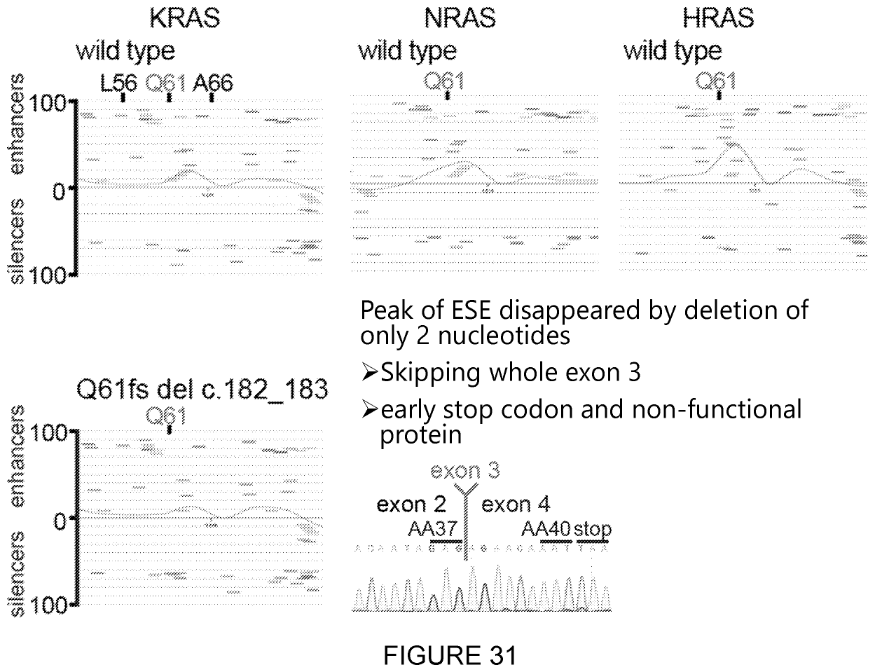

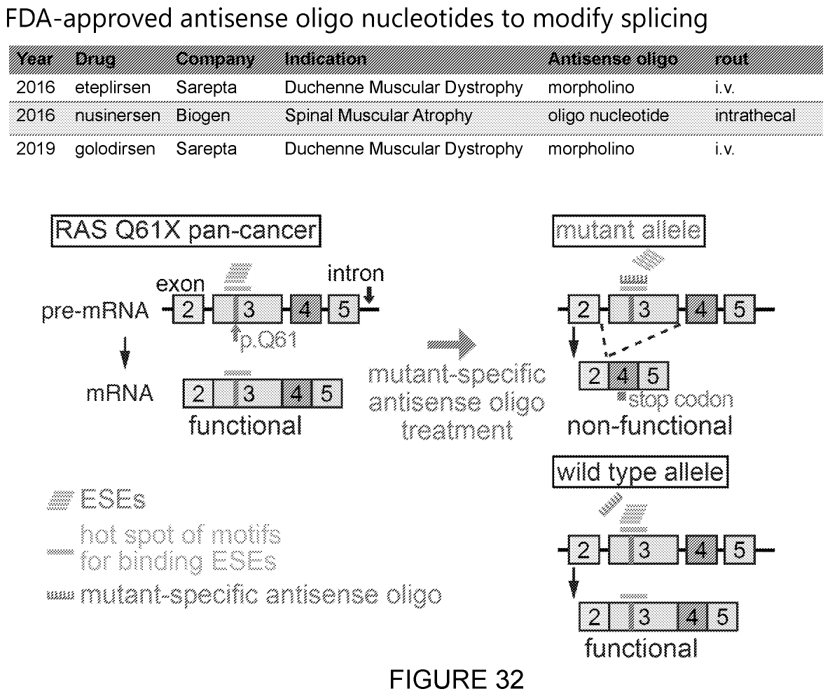

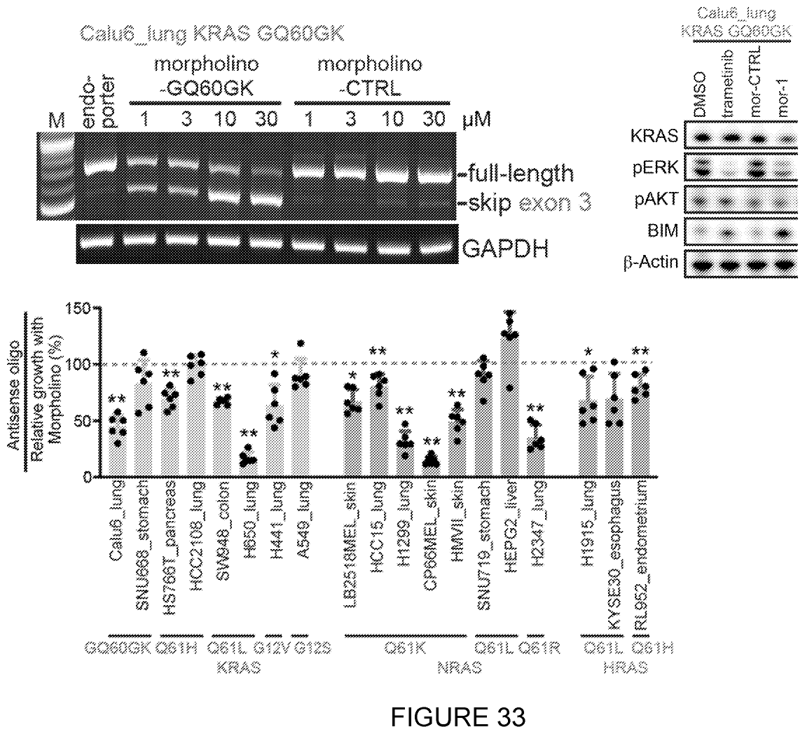

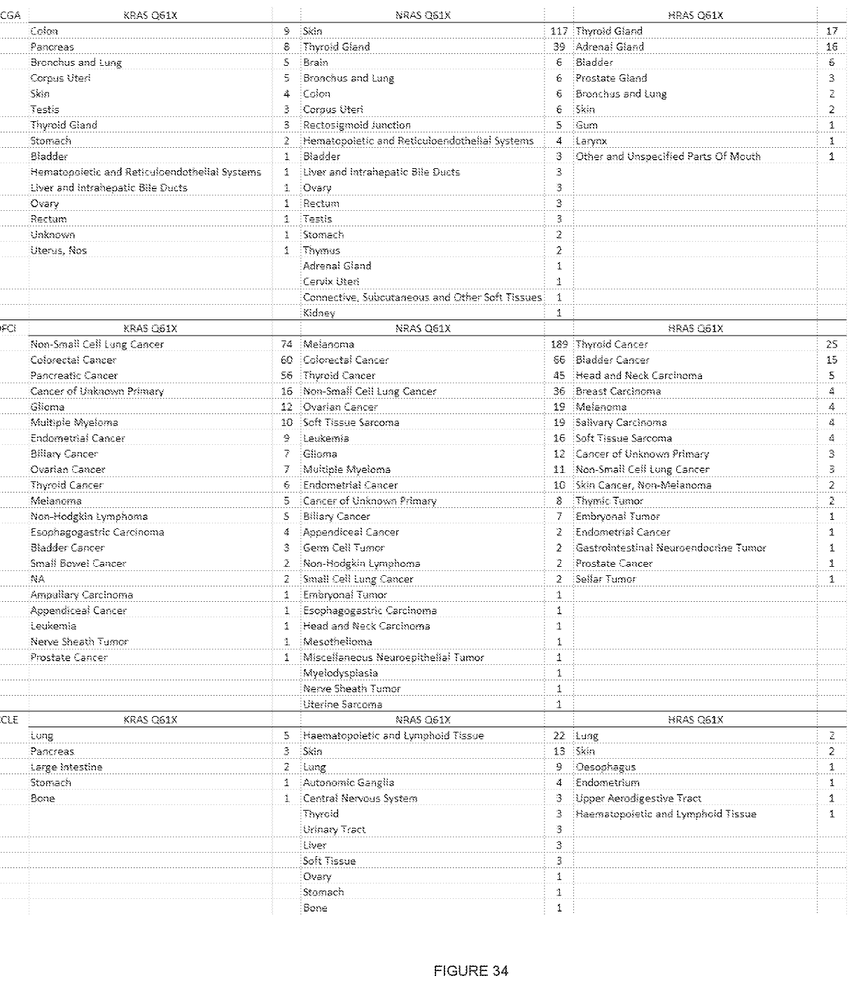

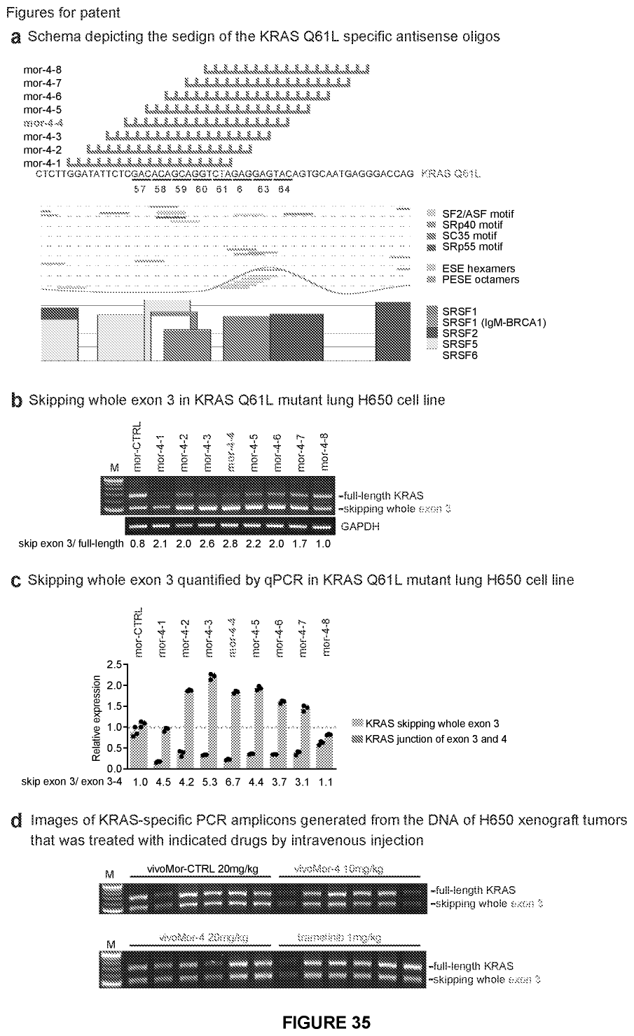

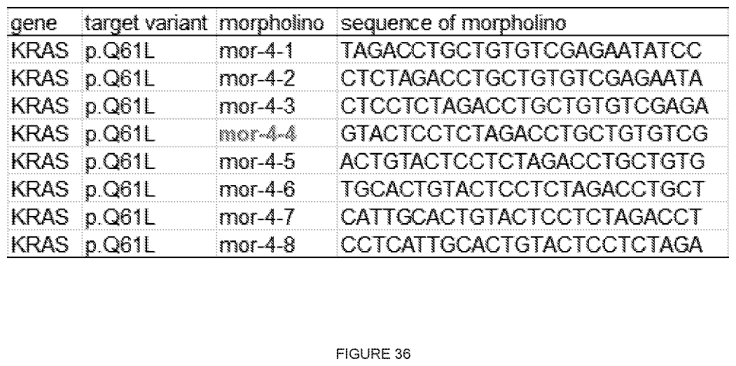

To create KRAS or BRAF mutations in PC9 cell lines, sgRNAs and donor templates for HDR were designed using Deskgen (deskgen.com). crRNAs (Integrated DNA Technologies, IDT) were hybridized with tracrRNAs to make 150 pmol sgRNAs, and then ribonucleoprotein complex was formed with 120 pmol Cas9 Nuclease (IDT) in vitro. The reaction mixtures and 120 pmol donor templates were nucleofected into PC9 cells (1×10⁵ cells) suspended in 20 μl of SE solution (IDT) using Lonza 4D-Nucleofector (Lonza) with pre-set PC9 mode. Cells were cultured in growth media with 30 μM Alt-R HDR Enhancer (IDT) for 12 hours. DNA was extracted from single clones using the DNeasy Mini kit (Qiagen) and mutations were confirmed by Sanger sequencing (Genewiz) or CRISPR sequencing at the Massachusetts General Hospital (MGH) DNA sequencing core. All sgRNAs, donor templates, and primers are listed in FIG. 24.

### Lentiviral Transfection

Firefly Luciferase Lentivirus (1.5×10⁶ CFU, Karafast) was used to transduce the H650 cell line (1.5×10⁵ cells) in the presence of polybrene (5 ug/ml, Santa Cruz Biotechnology), followed by centrifugation at 1,200×g for 90 minutes at 32° C., and then cultured for 12 hours at 37° C. Luciferase-expressing cells were selected in 1 ug/ml puromycin (Thermo Fisher) for 5 days.

### Colony Formation Assay and Allele Frequency Evaluation

Bulk PC9 cells edited to contain KRAS or BRAF mutations (1×10⁵ cells) were seeded into 12-well plates and cultured with or without 30 nM osimertinib. After staining with 0.5% crystal violet in 25% methanol for 30 minutes, images were taken. To evaluate allele frequency of specific mutant over time, DNA was extracted from bulk edited cells and submitted to CRISPR sequencing (MGH DNA core).

Gene Knock-Down by siRNA

Control siRNA or target-specific siRNA (final concentration of 10 nM, Life Technologies) and Lipofectamin RNAiMAX Transfection Reagent (final concentration of 0.3%, Thermo Fisher) were mixed in Opti-MEM (Gibco). After 10 minutes, the mixture was added into CRISPR-modified PC9 cell lines with growth media. For growth-inhibition assay, cells were trypsinized 24 hours after transfection, and cultured in 384-well plates for 24 hours, then treated with osimertinib (FIG. 1 panel h). For western blot analysis, samples were collected 48 hours after transfection (FIG. 5 panel c). In experiments using RAS mutant cell lines, control siRNA or indicated SMARTpool siRNA (final concentration of 25 nM, Dharmacon) and DharmaFECT1 or DharmaFECT2 (final concentration of 0.3%, Dharmacon) were mixed in Opti-MEM for 5 minutes, and then the reaction mixtures were added to cells in growth media (FIG. 4 panel d).

### Cell Growth-Inhibition Assay

Parental or CRISPR-modified PC9 cell lines (1×10³ cells) were plated in 384-well plates. After 24 hours, cells were treated with drugs at the indicated concentrations for 72 hours. Endpoint cell viability assays were performed using Cell Titer Glo (Promega). RAS mutant cell lines (2×10³ cells) were seeded in 384 ultra-low attachment plates as suspension cells and evaluated using 3D-Cell Titer Glo (Promega). RAS mutant cells were treated with trametinib for 3 days and with antisense oligos or siRNA for 8 days.

### RAS Isoforms

RNA was extracted using the RNeasy Mini kit (Qiagen) and cDNA was synthesized using the QuantiTect Reverse Transcription Kit (Qiagen). Isoforms were amplified using gene-specific-primers (FIG. 24) and amplicons were resolved on a 2% agarose gel. Isoforms were characterized by Sanger sequencing.

### Quantitative RT-PCR

The qPCR reactions were set up in 20 μl using TaqMan Gene Expression Master Mix (Thermo Fisher) including 1 ul of 1:5 diluted cDNA synthesized from 1 ug RNA. The reactions were run in StepOne Plus Real-time PCR System (Applied Biosystems). Expression levels of target genes were normalized to those of GUSB housekeeping gene in each sample. Primers and probes were designed to target exon 1 to 2 of normal KRAS isoform and isoform with skipping 112 bp of exon 3 (FIG. 24).

### Antibodies and Western Blot Analysis

Cells were lysed with RIPA buffer (Boston Bioproducts) supplemented with complete Mini EDTA-free Protease inhibitor cocktail (Roche) and PhoSTOP phosphatase inhibitor cocktail (Roche). The total cell lysate (20 ug) was subjected to SDS polyacrylamide gel electrophoresis and transferred to Immobilon-P polyvinylidene difluoride membranes (Bio-Rad Laboratories). Antibodies are listed in supplementary table 10. RAS-GTP was evaluated using the Active Ras Detection Kit (#8821, Cell Signaling Technology). Cells were cultured with media containing 0.1% FBS with or without 1 μM osimertinib for 24 hours, and 80 ug of GST-Raf1-RBD and 500 ug of protein lysates were used according to the manufacturer's instructions.

### Simulating Consensus Value of Splice Site

Consensus values of splice site were estimated by Human Splicing Finder²⁴. To evaluate distribution of exonic splicing enhancer (ESE) and silencer (ESS) sites around KRASINRAS/HRAS Q61, wild type and mutant sequences were simulated using the same Human Splicing Finder. For the purpose of designing antisense oligos, the locations of ESEs were also simulated using ESE finder^{38,39} to be validated by independent algorisms.

### Designing Antisense Oligos

Mutant-selective 25nt morpholino, vivo-morpholino (Gene Tools), and DNA with full PS+2′MOE modification (IDT) were designed against the region of ESE motifs simulated by Human Splicing Finder and ESE finder, and self-dimerization potential was estimated using Oligo Analyzer (https://www.idtdna.com/calc/analyzer/). Universal control 20nt antisense oligos were designed with 3 mismatches relative to the wild type sequence around KRAS, NRAS, and HRAS Q61 (FIG. 19).

Binding Affinity of Morpholinos with Mutant or Wild Type Sequences

Predicted binding affinity of morpholinos designed against mutant or wild type sequences were calculated using the UNAFold Web Server with a setting of Na 50 mM, Mg 1.2 mM, and oligo 0.25 μM (unafold.org/Dinamelt/applications/hybridization-of-two-different-strands-of-dna-or-rna.php).

Treatment with Morpholino or Antisense Oligo with PS+2′MOE In Vitro

RAS mutant cell lines in culture media were treated in suspension with indicated concentrations of morpholino and 3-6 μM endo-porter (Gene Tools). Duration of treatment was 2 days for RNA experiments, 3 days for western blot, and 8 days for growth-inhibition assay based on previously published treatment protocols with KRAS selective inhibitors against RAS mutant cells⁴. In all experiments using full PS+2′MOE antisense oligos, medium was enriched with Ca²⁺. Ca²⁺ enrichment of medium potentiates the in vitro activity of multiple types of oligonucleotides and is more reflective of in vivo conditions than conventional transfection methods⁴². Duration of treatment was 2 days for RNA experiments, 6 days for western blot, and 8 days for growth-inhibition assay.

### Pilot Study of Pre-Treatment Strategy Using Vivo-Morpholino In Vitro

Cells in suspension were treated with vivo-morpholinos without endo-porter in culture media containing 1% FBS, followed by morpholino oligo wash-out. Then, cells were seeded into ultra-low attachment plates and cultured with complete media for 8 days until 3D-Cell Titer Glo assay.

### In Vivo Efficacy Study

Luciferase-expressing H650 and Calu6 cell lines were pre-treated with control vivo-morpholino or targeting vivo-morpholino without endo-porter in culture media containing 1% FBS for 1 or 2 days. After morpholino oligo wash-out, same number of viable cells (5×10⁶ cells) with 50% Matrigel (Fisher Scientific) were implanted subcutaneously in the right flank of the NSG mice. The tumor burden was assessed by bioluminescent imaging beginning from day 2 using IVIS Spectrum (Perkin Elmer) at least twice weekly. Tumor volumes were also measured using caliper measurements at least twice weekly. Total bioluminescence was measured as photons/sec/cm²/sr and the tumor volumes were determined by using the formula, Tumor volume=(length×width²)/2. Body weights were measured twice weekly. For intra-tumoral injection experiments, 0.5 mM vivo-morpholino reconstituted in PBS were injected daily. Tumor samples were collected to evaluate pharmacodynamics at 4 hours after day 7 of drug administration.

### Clinical Data

Non-synonymous and silent mutations in KRAS, NRAS, and HRAS genes were obtained from The Cancer Genome Atlas (TCGA) pan-cancer cohort. Pan-cancer cohort (n=25,252) evaluated by targeted-next generation sequencing Oncopanel⁴³ at the Dana-Farber Cancer Institute was queried and de-identified KRAS Q61K and KRAS G60G data were extracted. We performed a retrospective review of the Guardant Health de-identified database to identify KRAS Q61K, Q61H, and G60G mutation positive patients with advanced stage solid tumors who had cell free DNA sequencing as part of standard clinical care between March 2014 and November 2019. Testing was performed in a Clinical Laboratory Improvement Amendments (CLIA)-certified, College of American Pathologists (CAP)-accredited, New York State Department of Health-approved clinical laboratory at Guardant Health, Inc. Analysis was completed under an Advarra Review Institution Review Board protocol for de-identified and limited datasets and did not require specific patient consent. Plasma was analyzed per methods previously described⁴⁴. We analyzed all reported genomic alterations from this cohort and performed a manual sequencing review for a subset of identified samples.

### Statistical Analysis

Mean values were assessed using unpaired two-tailed Student's t-test or ANOVA followed by Dunnett's post-hoc test. The correlation was analyzed by Pearson's correlation coefficient. The frequency of co-occurrence of activating non-synonymous Q61X and the G60G silent mutation was evaluated by Fisher's exact test. Columns represent means±standard deviation. Student's t-tests were used to compare the tumor volume/relative bioluminescence at last experimental time point and linear mixed models with random slopes were applied to compare the growth rate between treatment groups. Asterisks used to indicate significance correspond with: *p<0.05, **p<0.01. GraphPad Prism9 and SAS 9.4 were used for all statistical analyses.

### METHOD REFERENCES

42 Hori, S.-i. et al. Ca2+ enrichment in culture medium potentiates effect of oligonucleotides. Nucleic Acids Research 43, e128-e128, doi: 10.1093 nar gkv626 (2015).
43 Garcia, E. P. et al. Validation of OncoPanel: A Targeted Next-Generation Sequencing Assay for the Detection of Somatic Variants in Cancer. Arch Pathol Lab Med 141, 751-758, doi: 10.5858 arpa. 2016-0527-OA (2017).
44 Odegaard, J. I. et al. Validation of a Plasma-Based Comprehensive Cancer Genotyping Assay Utilizing Orthogonal Tissue-and Plasma-Based Methodologies. Clin Cancer Res 24, 3539-3549, doi: 10.1158/1078-0432.CCR-17-3831 (2018).

### Example 3

FIG. 4, panel d shows dotted lines which highlights the baseline ratio of mutant to wild type KRAS transcript frequency detected in the untreated SW984 cell line. In the absence of any selectivity of mor-4 for the mutant over wild type allele, the absolute number of transcript reads of each allele would decrease by the same magnitude, therefore maintaining the relative ratio of the mutant to wild type transcript frequency as would be observed with no treatment (indicated by the dotted line). When any degree of selectivity is observed for the mutant allele, however, the absolute count of transcript reads of the mutant would decrease by a greater degree than that of the wild type, effectively decreasing the mutant to wild type frequency ratio. This can be shown in the mutant frequency falling below the dotted line.

Importantly, we used a second type of antisense oligo, PS+2′MOE. In order to decrease the effective dose, we improved the delivery of the oligos into cells by shortening the original 25nt PS+2′MOE to 20nt. We screened 6 different 20nt PS+2′MOE, each varying in 3 nucleotides, in order to identify one that can efficiently and selectively covers the hotspot of exonic splicing enhancers (ESEs). The designed PS+2′MOE oligos (PS+MOE 1-3; highlighted in red in FIG. 14) achieved mutant selective skipping at a much lower concentration, as compared with previously used 25nt morpholino (FIG. 14 panels a-f). Binding affinity and off-target of newly designed PS+2′MOE oligos are also shown in FIG. 22 and FIG. 23).

We show (FIG. 14 panels a-f) selective antisense oligos (PS+2′MOE oligos) with improved efficacy, which, without wishing to be bound by theory, will result in improvements in their in vivo activity profile.

Data indicating the efficacy of morpholinos in KRAS mutant cells but not in KRAS wild type PC9 cells (EGFR mutant) nor normal BEAS2B cell (human bronchial cells) (FIG. 10 panel a).

We designed a new set of shorter 20nt PS+2′MOE antisense oligos to improve cellular delivery and mutant selectivity. The selected set of newly designed oligos induced exon skipping at a much lower concentration (0.03 or 0.1 μM) compared to the original morpholinos (10 μM), and achieved a therapeutic window where exon skipping was induced only in Q61 mutant cells but not in wild type cells (FIG. 14 panels a-f, FIGS. 22 and 23). The efficacy of PS+2′MOE antisense oligos were evaluated in both KRAS mutant and wild type PC9 cells (FIG. 14 panel f).

Despite the difference of only 1 or 2nt between the mutant and wild type sequence, the mean difference in predicted affinity (Tm) between morpholinos against the mutant or wild type allele was 4.8 (1.8-9.3) degrees (Fig). Additionally, the designed short 20nt PS+2′MOE antisense oligos display Tm differences of 12.2 and 4.1 degrees (FIG. 22) thus indicating that even small differences can lead to differences that result in selective mutant over WT alleles. This is further supported by the additional growth inhibition data in KRAS mutant and WT cells (FIG. 10, panel a, and FIG. 14).

Furthermore, mutant selective exon skipping was achieved at lower concentrations than with the morpholino oligos. Without wishing to be bound by theory, these findings validate mutant selective exon skipping and, as a consequence, a therapeutic window using 20nt PS+2′MOE oligos.

We developed and evaluated shorter PS+2′MOE oligos which induced exon skipping at a much lower concentration compared with the morpholino oligos (FIG. 10, panel a, and FIG. 14). Our data indicate that optimization of antisense oligos can improve efficacy and selectivity, diminishing toxic effects seen with high doses of the original morpholinos.

We analyzed splicing patterns using RNA sequencing data derived from a TCGA cohort using the TCGA SpliceSeq tool (https://bioinformatics.mdanderson.org/public-software/tcgaspliceseq/).

Exon 3 skipping was found in 1 out of 7 cases with KRAS GQ60GK pan-cancer cohort as shown FIG. 18. Since this case was colon adenocarcinoma, we subsequently investigated the frequency of exon 3 skipping in KRAS G12D mutant colon cancer cohort (n=49) and KRAS G12C mutant lung adenocarcinoma cohort (n=58) as a control. Overall, exon 3 skipping was detected in approximately 10% of each cohort with a low fraction (range 3-7%) of exon 3 skipping. Overall, our findings indicate that a minority of cancers have a natural proclivity to splice out exon 3 at baseline.

We have evaluated 20nt PS+2′MOE oligos and demonstrate an enhanced mutant selectivity at lower concentrations than possible with morpholino oligos (FIG. 14). Even for the morpholino oligos, there is cellular mutant selectivity in growth inhibition (Figure FIG. 10, panel a) with no effects observed in growth of EGFR mutant (PC9 cells) or normal cells (BEAS2B; normal bronchial epithelial cells). Collectively these findings indicate that it is possible to achieve mutant selectivity in exon 3 skipping and in growth inhibition using the approaches described herein.

To model resistance to the EGFR tyrosine kinase inhibitor osimertinib, we introduced specific oncogenic KRAS and BRAF mutations into EGFR mutant PC9 NSCLC cells via CRISPR-Cas9-mediated homology directed repair. In embodiments, we monitored allele frequency (AF) in the resulting clones under drug selection pressure, and observed that AF of KRAS G12C, G12D and A146T mutants increased whereas Q61K did not. In embodiments, Q61K AF increased only when accompanied by a concurrent silent mutation at G60 (e.g., GQ60GK c.180_181 delinsAA, CA or GA), which can be due to errors in NON-homologous end joining repair. Further, clones expressing Q61K but lacking an accompanying silent G60G mutation failed to induce osimertinib expression, as can be if Q61K were expressed and functional. These observations led to the surprising finding that the KRAS Q61K mutant uniquely possesses a previously unappreciated splice donor site that leads to a frameshift and loss of part or all of exon 3, resulting in translation of a truncated, dysfunctional protein. The presence of an adjacent nonsynonymous mutation at G60 converts the GU motif to GA or GC and therefore a nonfunctional splice site, which enables correct splicing of Q61K and expression of full length, functional protein.

Examination of 3 separate large databases of pan-cancer tumor genomes showed a truly striking correlation between the presence of a Q61K mutation and the presence of silent G60G mutations.

In embodiments, we were able to identify two types of antisense oligos that were capable of selectively causing exon skipping and growth inhibition both in vitro and in vivo, thereby providing a therapeutic strategy for Q61 mutant cancers.

That this requirement for a silent mutation is unique to KRAS Q61K, but not other Q61 mutants (e.g., Q61H) and not NRAS or HRAS isoforms, all of which have distinct sequences that lack the cryptic splice, is further support for the notion that proper expression of KRAS Q61K requires the concurrent presence of the accompanying silent G60G mutation specifically because this enables correct splicing of Q61K.

This finding is important for multiple reasons:
1) it reveals a regulation of sequence-specific oncogenic activity that will be missed entirely by evaluations of expression and function that rely on ectopic expression of cDNAs that are of already-spliced transcripts, which is standard across many fields, not only for RAS. (Even Sharma et al., Nat Commun 2019 [ref 37], in a study that indicated that patient-derived synonymous mutations in KRAS can alter mRNA structure and protein expression, confirmed their findings by introduction of specific single mutations into plasmids containing only coding sequences, and did not observe the striking truncation shown here that was a result of exon skipping);
2) it provides an explanation for the relative infrequency of KRAS Q61K mutations that is distinct from the generally assumed but not always demonstrated explanatory differences in disease etiologies, or structural, biochemical or biological functions of the protein;
3) without wishing to be bound by theory, short C-terminally truncated Q61K protein that is generated when the splice is not disrupted, i.e., when Q61K is not accompanied by the silent G60G mutation, is not only not fully functional, but is also a dominant negative. This is supported by analogy to the known dominant negative activity of lab-generated, C-terminally defective or truncated HRAS Q61L mutants and others, as well as by the data in this manuscript's FIG. 1 panel g (decreased pERK and failure of rebound after drug treatment in cells expressing Q61K without synonymous G60G);
4) it provides a means to block expression of KRAS Q61K in a mutant-selective and therefore less toxic manner than the majority of existing anti-RAS therapies, and
5) it led to a successful search for other mutant-selective Q61X oligos, by focusing on the exonic splicing enhancer (ESE)-rich environment of the Q61 sequence in different RAS isoforms, thereby extending the therapeutic reach of such an approach.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A nucleic acid comprising a sequence that hybridizes to a target, wherein the target comprises a precursor-mRNA (pre-mRNA) of a RAS family gene, wherein the precursor-mRNA comprises an exon splicing enhancer (ESE) binding motif comprising at least one mutation.

2. The nucleic acid of claim 1, wherein the nucleic acid modulates splicing of the pre-mRNA.

3. The nucleic acid of claim 2, wherein the nucleic acid promotes splicing or inhibits splicing of the pre-mRNA.

4. The nucleic acid of claim 1, wherein the target comprises exon 3 of the pre-mRNA or portion thereof.

5. The nucleic acid of claim 4, wherein the nucleic acid promotes alternative splicing which excludes exon 3 or a portion thereof.

6. The nucleic acid of claim 1, wherein the sequence is at least partially complementary to, partially complementary to, or fully complementary to the target.

7. The nucleic acid of claim 1, wherein the target comprises at least one mutation as compared to wildtype pre-mRNA.

8. The nucleic acid of claim 1, wherein the target comprises the exon splicing enhancer (ESE) binding motif, or a sequence adjacent thereto.

9. The nucleic acid of claim 1, wherein the sequence is not complementary to wildtype pre-mRNA.

10. The nucleic of claim 1, wherein the RAS family gene comprises KRAS, NRAS, and HRAS.

11. The nucleic acid of claim 1, wherein the exon splicing enhancer (ESE) binding motif comprises codons 52 to 70 of the pre-mRNA or a portion thereof.

12. The nucleic acid of claim 11, wherein the exon splicing enhancer (ESE) binding motif comprises codons 55 to 65 of pre-mRNA of KRAS, or a portion thereof.

13. The nucleic acid of claim 11, wherein the exon splicing enhancer (ESE) binding motif comprises codons 55 to 66 of pre-mRNA of NRAS.

14. The nucleic acid of claim 11, wherein the exon splicing enhancer (ESE) binding motif comprises codons 58 to 67 pre-mRNA of HRAS.

15. The nucleic acid of claim 1, wherein the target comprises a sequence comprising
[SEQ ID NO: []]
5′-CUC UUG GAU AUU CUC GAC
ACA GCA GGX₁ X₂X₃X₄ GAG GAG
UAC AGU GCA AUG AGG GAC CAG-3′,
a fragment thereof,
or a sequence that is at least 90% identical thereto.

16. The nucleic acid of claim 15, wherein
X₁ is G, A, U, or C;
X₂ is G, A, U, or C;
X₃ is G, A, U, or C;
X₄ is G, U, A, or C; or
any combination thereof.

17. The nucleic acid of claim 15, wherein the sequence comprises:
[SEQ ID NO: []]
5′-CUC UUG GAU AUU CUC GAC
ACA GCA GGU CUA GAG GAG
UAC AGU GCA AUG AGG GAC CAG-3′,
[SEQ ID NO: []]
5′-CUC UUG GAU AUU CUC GAC
ACA GCA GGU CAC GAG GAG
UAC AGU GCA AUG AGG GAC CAG-3′,
[SEQ ID NO: []]
5′-CUC UUG GAU AUU CUC GAC
ACA GCA GGG AAA GAG GAG UAC
AGU GCA AUG AGG GAC CAG-3′,
and
[SEQ ID NO: []]
5′-CUC UUG GAU AUU CUC GAC
ACA GCA GGC AAA GAG GAG UAC
AGU GCA AUG AGG GAC CAG-3′,
or a sequence at least 90% identical thereto.

18. The nucleic acid of claim 1, wherein target comprises a sequence comprising
[SEQ ID NO: []]
5′-UUG UUG GAC AUA CUG GAU
ACA GCU GGA X₁X₂A GAA GAG
UAC AGU GCC AUG AGA GAC
CAA-3′,
a fragment thereof,
or a sequence that is at least 90% identical thereto.

19. The nucleic acid of claim 18, wherein
X₁ is G, U, A, or C;
X₂ is G, U, A, or C; or
any combination thereof.

20. The nucleic acid of claim 18, wherein the sequence comprises:
[SEQ ID NO: []]
5′-UUG UUG GAC AUA CUG GAU
ACA GCU GGA CGA GAA GAG
UAC AGU GCC AUG AGA GAC
CAA-3′,
[SEQ ID NO: []]
5′-UUG UUG GAC AUA CUG GAU
ACA GCU GGA AAA GAA GAG
UAC AGU GCC AUG AGA GAC
CAA-3′,
[SEQ ID NO: []]
5′-UUG UUG GAC AUA CUG GAU
ACA GCU GGA CUA GAA GAG
UAC AGU GCC AUG AGA GAC
CAA-3′,
or a sequence at least 90% identical thereto.

21. The nucleic acid of claim 1, wherein the target comprises a sequence comprising:
[SEQ ID NO: []]
5′-CUG UUG GAC AUC CUG GAU
ACC GCC GGC CXX GAG GAG
UAC AGC GCC AUG CGG GAC
CAG-3′,
a fragment thereof,
or a sequence at least 90% identical thereto.

22. The nucleic acid of claim 21, wherein:
X₁ is G, U, A, or C;
X₂ is G, U, A, or C; or
any combination thereof.

23. The nucleic acid of claim 21, wherein the sequence comprises:
[SEQ ID NO: []]
5′-CUG UUG GAC AUC CUG GAU
ACC GCC GGC CUG GAG GAG
UAC AGC GCC AUG CGG GAC
CAG-3′,
[SEQ ID NO: []]
5′-CUG UUG GAC AUC CUG GAU
ACC GCC GGC CAU GAG GAG
UAC AGC GCC AUG CGG GAC
CAG-3′,
or a sequence at least 90% identical thereto.

24. The nucleic acid of claim 1, wherein the at least one mutation comprises a mutation within codon 61.

25. The nucleic acid of claim 1, wherein the at least one mutation comprises G60X, Q61X, or a combination thereof.

26. The nucleic acid of claim 25, wherein the at least one mutation comprises Q61H, Q61L, Q61R, or Q61K.

27. The nucleic acid of claim 25, wherein the at least one mutation comprises GQ60GK.

28. The nucleic acid of claim 1, wherein the nucleic acid comprises a sequence according to
[SEQ ID NO: []]
5′-GTACTCCTCX₁X₂X₃X₄CCTGCTGTGTCG-3′,
a sequence that is at least 90% identical thereto.

29. The nucleic acid of claim 28, wherein:
X₁ is G or T;
X₂ is A or T;
X₃ is G or T;
X₄ is G, C, or A; or
any combination thereof.

30. The nucleic acid of claim 28, wherein the nucleic acid comprises a sequence of
[SEQ ID NO: []]
5′-GTACTCCTCTTTGCCTGCTGTGTCG-3′,
[SEQ ID NO: []]
5′-GTACTCCTCTTTCCCTGCTGTGTCG-3′,
[SEQ ID NO: []]
5′-GTACTCCTCGTGACCTGCTGTGTCG-3′,
[SEQ ID NO: []]
5′-GTACTCCTCTAGACCTGCTGTGTCG-3′,
or
a sequence that is at least 90% identical thereto.

31. The nucleic acid of claim 1, wherein the nucleic acid comprises a sequence according to
[SEQ ID NO: []]
5′-CTTX₁X₂CCTGCTGTGTCGAGA-3′,
or
a sequence that is at least 90% identical thereto.

32. The nucleic acid of claim 31, wherein:
X₁ is G or T;
X₂ is G, C or A; or
any combination thereof

33. The nucleic acid of claim 31, wherein the nucleic acid comprises a sequence of
[SEQ ID NO: []]
5′-CTTTGCCTGCTGTGTCGAGA-3′,
[SEQ ID NO: []]
5′-CCTCTTTGCCTGCTGTGTCG-3′,
[SEQ ID NO: []]
5′-ACTCCTCTTTGCCTGCTGTG-3′,
[SEQ ID NO: []]
5′-TGTACTCCTCTTTGCCTGCT-3′,
[SEQ ID NO: []]
5′-CACTGTACTCCTCTTTGCCT-3′,
or
[SEQ ID NO: []]
5′-TTGCACTGTACTCCTCTTTG-3′.

34. The nucleic acid of claim 1, wherein the nucleic acid comprises a sequence according to 5′-X₁X₂X₃X₄-3′, wherein X₁ is G or T; X₂ is A or T; X₃ is G or T; X₄ is G, C, or A; or any combination thereof.

35. The nucleic acid of claim 34, wherein the nucleic acid further comprises 5′ flanking nucleotides, 3′ flanking nucleotides, or both 5′ flanking nucleotides and 3′ flanking nucleotides.

36. The nucleic acid of claim 35, wherein the nucleic acid comprises a sequence of
(SEQ ID NO: [])
5′-TAGACCTGCTGTGTCGAGAATATCC-3′,
(SEQ ID NO: [])
5′-CTCTAGACCTGCTGTGTCGAGAATA-3′,
(SEQ ID NO: [])
5′-CTCCTCTAGACCTGCTGTGTCGAGA-3′,
(SEQ ID NO: [])
5′-GTACTCCTCTAGACCTGCTGTGTCG-3′,
(SEQ ID NO: [])
5′-ACTGTACTCCTCTAGACCTGCTGTG-3′,
(SEQ ID NO: [])
5′-CATTGCACTGTACTCCTCTAGACCT-3′,
or
(SEQ ID NO: [])
5′-CCTCATTGCACTGTACTCCTCTAGA-3′.

37. The nucleic acid of claim 1, wherein the nucleic acid comprises a sequence of
[SEQ ID NO: []]
5′-CTCTTCTX₁X₂TCCAGCTGTATCCAGT-3′,
or a sequence that is at least 90% identical thereto.

38. The nucleic acid of claim 37, wherein:
X₁ is C, T, or A;
X₂ is T or G;
or any combination thereof.

39. The nucleic acid of claim 37, wherein the nucleic acid comprises a sequence of
[SEQ ID NO: []]
5′-CTCTTCTCGTCCAGCTGTATCCAGT-3′,
[SEQ ID NO: []]
5′-CTCTTCTTTTCCAGCTGTATCCAGT-3′,
[SEQ ID NO: []]
5′-CTCTTCTAGTCCAGCTGTATCCAGT-3′,
or
a sequence that is at least 90% identical thereto.

40. The nucleic acid of claim 1, wherein the nucleic acid comprises a sequence of
[SEQ ID NO: []]
5′-TGGCGCTGTACTCCTCX₁X₂GGCCGG-3′,
or a sequence that is at least 90% identical thereto.

41. The nucleic acid of claim 40, wherein:
X₁ is A or C;
X₂ is T or A;
or any combination thereof.

42. The nucleic acid of claim 40, wherein the nucleic acid comprises a sequence of
[SEQ ID NO: []]
5′-TGGCGCTGTACTCCTCCAGGCCGG-3′,
[SEQ ID NO: []]
5′-TGGCGCTGTACTCCTCATGGCCGG-3′,
or
a sequence that is at least 90% identical thereto.

43. The nucleic acid of any one of claims 1-42, wherein the nucleic acid comprises a modified nucleic acid.

44. The nucleic acid of claim 43, wherein the modified nucleic acid comprises a sugar modification, a backbone modification, a base modification, an unnatural base pair, conjugation to a cell penetrating peptide, or any combination thereof.

45. The nucleic acid of claim 43, wherein the modification comprises phosphorothioate (PS)+2′-O-Methyoxyethyl (2′MOE).

46. The nucleic acid of claim 43, wherein the modified nucleic acid is a morpholino, a locked nucleic acid (LNA), amido-bridged nucleic acid (AmNA), or peptide nucleic acid (PNA).

47. The nucleic acid of claim 46, wherein the morpholino is a cell penetrating peptide-conjugated morpholino.

48. A composition comprising a nucleic acid according to any one of claims 1-47.

49. The composition of claim 48, wherein the composition further comprises a pharmaceutically acceptable carrier, diluent, or excipient.

50. The composition of claim 48, further comprising at least one additional active agent.

51. The composition of claim 50, wherein the at least one additional active agent comprises an anti-cancer agent.

52. A method for treating cancer in a subject in need thereof, the method comprising administering to the subject a therapeutically effective amount of a nucleic acid according to any one of claims 1-47 or a composition according to any one of claims 48-51.

53. The method of claim 52, wherein the cancer comprises a RAS-associated cancer.

54. The method of claim 53, wherein the RAS is KRAS, NRAS, or HRAS.

55. The method of claim 53, wherein the RAS-associated cancer comprises at least one mutation in KRAS, NRAS, or HRAS.

56. The method of claim 55, wherein the at least one mutation comprises G60X, Q61X, or a combination thereof.

57. The method of claim 56, wherein the at least one mutation comprises Q61H, Q61L, Q61R, or Q61K.

58. The method of claim 56, wherein the at least one mutation comprises GQ60GK.

59. A method for inducing splicing in a subject, the method comprising administering to the subject a therapeutically effective amount of a nucleic acid according to any one of claims 1-47 or a composition according to any one of claims 48-51.

60. A method for modulating splicing of a RAS pre-mRNA in a tumor cell, the method comprising contacting the tumor cell with the nucleic acid according to any one of claims 1-47 in an amount effect to modulate splicing of the RAS pre-mRNA in the tumor cell.

61. The method of claim 60 wherein modulating comprises enhancing or inducing splicing.

62. The method of claim 60, wherein modulating splicing comprises modulating splicing of Exon 3.

63. The method of claim 60, wherein splicing is modulated in tumor cells but not in normal cells.

64. A method for inducing skipping of an exon in a tumor cell, the method comprising contacting the tumor cell with the nucleic acid according to any one of claims 1-47 in an amount effective to induce skipping of an exon of in the tumor cell.

65. The method of claim 64, wherein the exon comprises an exon of RAS.

66. The method of claim 65, wherein the exon comprises exon 3.

67. The method of claim 65, wherein the RAS comprises KRAS, NRAS, or HRAS.

68. A kit comprising a nucleic acid according to any one of claims 1-47 and instructions for use thereof.

69. A kit comprising the composition according to any one of claims 48-51 and instructions for use thereof.
